(19) United States
(12) Patent Application Publication
(10) Pub. No.: US 2025/0050668 A1
(43) Pub. Date: Feb. 13, 2025

(54) TERRYLENE DIIMIDE AND QUATERRYLENE DIIMIDE COLORANTS

(71) Applicant: BASF SE, Ludwigshafen am Rhein (DE)

(72) Inventors: Hannah Stephanie MANGOLD, Ludwigshafen am Rhein (DE); Martin KOENEMANN, Ludwigshafen am Rhein (DE); Sorin IVANOVICI, Vienna (AT)

(21) Appl. No.: 18/716,575

(22) PCT Filed: Dec. 9, 2022
(86) PCT No.: PCT/EP2022/085121

§ 371 Date: Jun. 5, 2024

(30) Foreign Application Priority Data
Dec. 9, 2021 (EP) 21213300.3

(51) Int. Cl. B41M 3/14; C07D 471/06; C09D 11/02

(52) U.S. Cl. CPC B41M 3/144; C07D 471/06; C09D 11/02

(57) ABSTRACT

The present invention relates to terrylene diimides which are substituted in the skeleton and their homologous quaterrylene diimides. The present invention also relates to specific uses of these compounds, especially to their use in an ink formulation for security printing applications, and to printing ink formulations for security printing and security documents comprising such compounds.

The present invention relates to terrylene diimides which are substituted in the skeleton and their homologous quaterrylene diimides. The present invention also relates to the use of these compounds, especially their use in an ink formulation for security printing applications.

### TECHNICAL BACKGROUND

Visible light absorbers and infrared (IR) light absorbers meet a significant technical need in a wide range of applications, such as security printing (bank notes, credit cards, identity cards, passports etc.), invisible and/or IR readable barcodes and specifically fluorescent barcodes (when the visible light absorbers and IR light absorbers show fluorescence), the laser-welding of plastics, the curing of surface-coatings using IR radiators, the drying and curing of prints, the fixing of toners on paper or plastics, optical filters for PDPs (plasma display panels), laser marking e.g. of paper or plastics, the heating of plastics preforms or heat shielding applications.

To prevent counterfeiting, marking is currently extensively used for the recogni-tion, identification and authentication of individual items. The application of markings is frequently carried out by a printing process which uses a printing ink with specific optical properties that are imparted to the ink by one or more substances contained therein such as, e.g., fluorescent dyes or pigments emitting in the IR wavelength range of the electromagnetic spectrum that can be detected upon excitation with an activating light of a suitable excitation wavelength spectrum; and/or colorless or barely colored IR absorbers that can be detected under IR light.

A further important application of IR absorbing dyes and fluorescent dyes is in liquids, especially fuel oils such as mineral oils, as marker. In this way, heating oil, which usually enjoys tax privileges, can be differentiated from the generally more highly taxed diesel oil.

A substance class which is suitable for marking purposes are the higher rylene-tetracarboximides, especially terrylenebis(dicarboximides) (also referred to as terrylene diimides) and quaterrylenebis(dicarboximides) (also referred to as quaterrylene diimides) because of their stability and strong absorption in the long-wavelength red and near-infrared region of the electromagnetic spectrum. The terrylenebis(dicarboximides) absorb electromagnetic radiation mainly in the visible range and generally emit at from 690 to 780 nm, while the higher rylene homolog quaterrylenebis(dicarboximides) principally have an absorption in the near infrared (NIR) and some of them also emit in the NIR. The representatives of this substance class may be substituted on the terrylene and quaterrylene core, respectively, by phenyl, phenoxy or thiophenoxy substituents which in turn may be monosubstituted, for example by tert-alkyl, or disub-stituted, for example by alkyl containing up to 4 carbon atoms.

WO 2007/099059 describes inter alia the use of terrylene and quaterrylene diimides that are substituted in the skeleton by alkyl-substituted phenoxy radicals as marker for liquids, especially oils such as mineral oils.

WO 2007/031446 describes fluorescence solar conversion cells based on aryloxy substituted terrylene dyes.

WO 2020/193309 describes the use of aryloxy-substituted terrylene diimides as wavelength conversion material for agriculture.

None of these documents describes terrylene or quaterrylene diimide compounds having the characteristic substituents attached at the skeleton as claimed in the present invention.

The performance of these terrylene and quaterrylene colorants is not always sat-isfactory. In particular, they often suffer from one or more of the following drawbacks: low solubility or dispersibility in liquid media such as those which are useful in printing inks—this low solubility/dispersibility limits the suitability of terrylene and quaterrylene compounds as colorants for liquids in general, also making the colorants unsuitable for detection —; low fluorescence quantum yield and/or low molar extinction coefficient, resulting in the article appearing more colored; loss of fluorescent properties by agglom-eration of fluorescent sites (quenching).

Further, for use in the field of security printing, it would be advantageous to provide a security image on an item requiring a security image which could be detected at wavelengths in the range from 690 to 1000 nm and in which a strongly coloured image is not created by the compound(s) utilised.

It was an object of the present invention to provide substituted terrylene diimides and quaterrylene diimides which have advantageous application properties and can be incorporated efficiently into the particular application medium. Preferably, the novel terrylene diimides and quaterrylene diimides should fulfil at least one of the following criteria:
(i) intense absorption in the wavelength range in the long-wavelength red and near infrared region of the electromagnetic spectrum (wavelength range from 690 to 1000 nm), i.e., high molar extinction coefficient;
(ii) intense emission in the wavelength range from 690 to 1000 nm, i.e., high fluorescence quantum yield;
(iii) zero or low absorption in the visible spectral range from 380 to less than 750 nm, especially a transmittance of at least 75% in the range of from 380 to less than 750 nm;
(iv) good solubility or dispersibility in liquid media such as a liquid medium comprised in a printing ink formulation;
(v) high compatibility with a number of formulations, in particular printing ink formulations used especially in security printing;
(vi) good adhesion to paper and other materials such as plastics;
(vii) good lightfastness;
(viii) good heat stability;
(ix) good fastness to boiling water;
(x) good fastness to acids and alkalines; and
(xi) low cost manufacture.

### SUMMARY OF THE INVENTION

It was surprisingly found that these and further objectives are achieved by the compounds of formula (I) as defined herein below.

Thus, in a first aspect, the invention relates to a compound of formula (I)

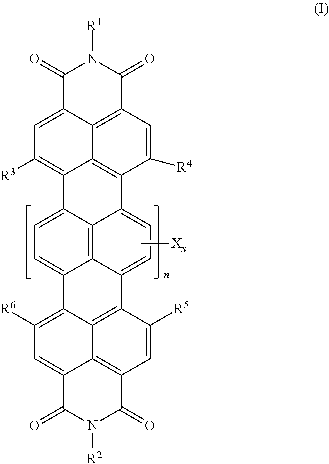

in which the substituents and indices are each defined as follows:
n is 1 or 2;
x is 0, 1 or 2;
each X is C₆-C₁₀-aryloxy or C₆-C₁₀-aryl, wherein C₆-C₁₀-aryloxy is substituted by one substituent R⁷ and one or more, e.g. one, two, three or four, identical or different substituents R^{7a}; and wherein C₆-C₁₀-aryl is substituted by one substituent R⁸ and zero, one or more, e.g. one, two, three or four, identical or different substituents R^{8a};
R¹ and R², independently of each other, are hydrogen, C₁-C₂₄-alkyl, C₁-C₂₄-haloalkyl, C₃-C₂₄-cycloalkyl, C₆-C₁₀-aryl or C₆-C₁₀-aryl-C₁-C₁₀-alkylene, where the rings of cycloalkyl, aryl, and aryl-alkylene in the three last-mentioned radicals are unsubstituted or substituted with one or more substituents R^{a}, and where C₁-C₂₄-alkyl, C₁-C₂₄-haloalkyl and the alkylene moiety of C₆-C₁₀-aryl-C₁-C₁₀-alkylene may be interrupted by one or more heteroatoms or heteroatomic groups selected from the group consisting of O, S and NR^{b};
R³, R⁴, R⁵ and R⁶, independently of each other, are C₆-C₁₀-aryloxy or C₆-C₁₀-aryl, wherein C₆-C₁₀-aryloxy is substituted by one substituent R⁷ and one or more, e.g. one, two, three or four, identical or different substituents R^{7a}; and wherein C₆-C₁₀-aryl is substituted by one substituent R⁸ and zero, one or more, e.g. one, two, three or four, identical or different substituents R^{8a};
R⁷ is C₆-C₂₀-alkyl;
R^{7a} is C₁-C₂₀-alkyl;
R⁸ is C₅-C₂₀-alkyl;
R^{8a} is C₁-C₂₀-alkyl;
each R^{a} is independently selected from group consisting of C₁-C₂₄-alkyl, C₁-C₂₄-fluoroalkyl, C₁-C₂₄-alkoxy, fluorine, chlorine and bromine; and
R^{b} is hydrogen, C₁-C₂₀-alkyl, C₃-C₂₄-cycloalkyl, heterocycloalkyl, hetaryl or C₆-C₁₀-aryl.

The compounds of formula (1) are substituted in the ring system either by aryloxy radicals substituted by one alkyl radical containing 6 to 20 carbon atoms and at least one further alkyl radical containing up to 20 carbon atoms, or they are substituted in the ring system by aryl radicals substituted by at least one alkyl radical having 5 to 20 carbon atoms, thereby rendering the compounds of formula (1) dispersible or soluble in a given application medium. The compounds of formula (1) are new, show a very strong absorbance in the wavelength range of from 690 to 1000 nm and fulfil at least one, preferably two, three, four or more than four of the above-mentioned criteria (ii) to (xi).

Thus, a further aspect of the present invention relates to the use of the compound of formula (1) as defined above and in the following in security printing, for coloring coatings, printing inks including printing inks for 3D printing and plastics, for data storage, for optical labels, for security labels in documents and for brand protection, for solar collectors, for optical waveguides, for the laser welding of plastics or as a fluorescent label for biomolecules.

A further aspect of the present invention relates to an article comprising at least one fluorescent film layer, the film layer comprising in a polymeric matrix the compound of formula (1) as defined above and in the following.

A further aspect of the present invention relates to a printing ink for security printing, comprising
a) at least one compound of formula (1) as defined above and in the following;
b) a polymeric binder;
c) optionally a solvent;
d) optionally at least one colorant;
e) optionally at least one further additive; and
f) optionally at least one photoinitiator.

A further aspect of the invention relates to a security document, comprising a substrate and the compound of formula (1) as defined above and in the following.

A further aspect of the invention relates to a security document, obtainable, preferably obtained, by a printing process, wherein a printing ink formulation is used that comprises the compound of formula (1) as defined above and in the following.

A further aspect of the invention relates to a method of detecting the authenticity of the security document as defined above and in the following, the method comprising the steps of:
a) measuring an absorbance, reflectance or transmittance spectrum of the security document in the visible/near infra-red range of the electromagnetic spectrum; and
b) comparing the spectrum measured under a) and/or information derived therefrom with a corresponding spectrum and/or information of an authentic security element.

### DETAILED DESCRIPTION OF THE INVENTION

Here and throughout the specification, the term “polymer matrix” refers to a polymer in which the compound of formula (1) is molecularly dissolved.

Here and throughout the specification, the term “fluorescence quantum yield” (QY) is defined as ratio of the number of photons emitted to the number of photons absorbed. Here and throughout the specification, substances which absorb electromagnetic radiation in the wavelength range of IR radiation are also referred to as IR absorbers or more specially as near infrared absorbers. IR absorbers preferably have an absorption in the wavelength range from 750 to 2000 nm, more preferably from 750 to 1000 nm (near-infrared light).

Here and throughout the specification, the term “zero or low absorption in the visible spectral range” is intended to mean that the absorber should ideally have no absorption at all in the visible spectral range. For the purposes of this invention, however, it is usually sufficient if the absorption of the absorber—in the chosen amounts—in the visible spectral range is so low that the color impression of the printing ink is not ad-versely affected. Of course, this also depends on the hue and on the color strength of the respective printing ink. An absorber which is no longer suitable for a printing ink having a very specific hue and a very specific color strength may in certain circum-stances be entirely suitable for another printing ink having another hue and another color strength.

Here and throughout the specification, the term “colorant” includes pigment, dye, mixtures of pigments, mixture of dyes and mixtures of pigments and dyes. The colorant is capable of absorbing light, the wavelength of maximum absorbance is either in the visible or in the infra-red.

Here and throughout the specification, colorants which absorb visible light are also referred to as colored absorber.

Here and throughout the specification, the term “visible light” denotes light that ranges from approximately 380 nm to less than 750 nm.

Here and throughout the specification, the term “near infra-red” (NIR) denotes light that ranges from 750 nm to 1000 nm.

Here and throughout the specification, the term “coating” means a film or layer applied on a substrate and dried and/or cured, i.e., final for use. The coating may comprise one or more layers.

Here and throughout the specification, the singular form “a”, “an” and “the” includes plural references unless the content clearly dictates otherwise. The term “a” (or “an”), in phrases such as “comprising a” means “at least one” and not “one and only one”. When the term “at least one” is specifically used, this should not be construed as having a limitation on the definition of “a”.

Here and throughout the specification, the term “compound of formula #” is also referred to as “formula #” or “compound #”.

Here and throughout the specification, the term “essentially” in the context of the polymeric material encompasses the terms “completely”, “wholly” and “all”. The term encompasses a proportion of 90% or more, such as 95% or more, especially 99% or 100%.

Here and throughout the specification, the term “halogen” denotes fluorine, bromine, chlorine or iodine, particularly chlorine, bromide or iodine Here and throughout the specification, the prefixes Cₙ-Cₘ used in connection with compounds or molecular moieties each indicate a range for the number of possible carbon atoms that a molecular moiety or a compound can have.

The term “C₁-Cₙ-alkyl” denotes a group of linear or branched saturated hydrocarbon radicals having from 1 to n carbon atoms. For example, the term C₁-C₂₀-alkyl denominates a group of linear or branched saturated hydrocarbon radicals having from 1 to 20 carbon atoms, while the term C₁-Cₐ-alkyl denominates a group of linear or branched saturated hydrocarbon radicals having from 1 to 4 carbon atoms, the term C₅-C₂₀ alkyl denominates a group of linear or branched saturated hydrocarbon radicals having from 5 to 20 carbon atoms and the term C₆-C₂₀-alkyl denominates a group of linear or branched saturated hydrocarbon radicals having from 6 to 20 carbon atoms. Examples of alkyl include but are not limited to methyl, ethyl, n-propyl, isopropyl, n-butyl, sec-butyl, isobutyl, 1,1-dimethylethyl (tert.-butyl), pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, 1,1,3,3-tetramethylbutyl (tert-octyl), nonyl, isononyl, decyl, undecyl, dodecyl, tridecyl, isotridecyl, tetradecyl, pentadecyl, hexadecyl, heptadecyl, octadecyl, nonadecyl, eicosyl, heneico-syl docosyl and in case of nonyl, isononyl, decyl, undecyl, dodecyl, tridecyl, isotridecyl, tetradecyl, pentadecyl, hexadecyl, heptadecyl, octadecyl, nonadecyl, eicosyl, heneico-syl, docosyl, their isomers, in particular mixtures of isomers such as “isononyl”, “isodecyl”. Examples of C₁-C₄-alkyl are for example methyl, ethyl, propyl, 1-methylethyl, butyl, 1-methylpropyl, 2-methylpropyl or 1,1-dimethylethyl.

The term “C₁-C₂₄-haloalkyl” as used herein denotes straight-chain or branched C₁-C₂₄ alkyl as defined above, where some or all of the hydrogen atoms in these groups are replaced by halogen atoms as mentioned above. Examples for C₁-C₂-haloalkyl are chloromethyl, bromomethyl, dichloromethyl, trichloromethyl, fluoromethyl, difluoromethyl, trifluoromethyl, chlorofluoromethyl, dichlorofluoromethyl, chlorodifluoro-methyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl.

The term “C₁-C₂₄-fluoroalkyl” as used herein denotes straight-chain or branched C₁-C₂₄ alkyl as defined above, where some or all of the hydrogen atoms in these groups are replaced by fluorine. Examples for C₁-C₂-fluoroalkyl are fluoromethyl, difluoromethyl, trifluoromethyl, 1-fluoroethyl, 2-fluoroethyl, 2,2-difluoroethyl, 2,2,2-trifluoroethyl and pentafluoroethyl.

The term “C₁-C₂₄-alkoxy” as used herein denotes straight-chain or branched C₁-C₂₄ alkyl as defined above bound to the remainder of the molecule through an oxygen. Examples for C₁-C₄-alkoxy are methoxy, ethoxy, n-propoxy, 1-methylethoxy, butoxy, 1-methylpropoxy, 2-methylpropoxy and 1,1-dimethylethoxy.

The term “C₃-C₂₄-cycloalky” as used herein denotes a mono-, bi- or tricyclic cycloalkyl radical. Examples of C₃-C₂₄-cycloalkyl include but are not limited to cyclopropyl, cyclobutyl, cyclopentyl, cyclohexyl, methylcyclohexyl, dimethylcyclohexyl, cycloheptyl, cyclooctyl, cyclododecyl, cyclohexadecyl and norbornyl (=bicyclo[2.2.1]heptyl).

The term “heterocyclyl” as used herein refers to a mono- or bicyclic saturated or partially unsaturated ring system having 3, 4, 5, 6, 7 or 8 ring members, comprising besides carbon atoms as ring members, one, two, three or four heteroatoms or heteroa-tom-containing groups selected from O, N, S, SO and S(O)₂ as ring members.

The term “C₆-C₁₀-aryl” as used herein denotes phenyl or naphthyl.

The term “C₆-C₁₀-aryloxy” as used herein denotes phenoxy and naphthyloxy.

The term “alkylene” or “alkanediyl” as used herein denotes a straight-chain or branched alkyl radical as defined above, wherein one hydrogen atom at any position of the carbon backbone is replaced by one further binding site, thus forming a bivalent moiety.

The term “C₆-C₁₀-aryl-C₁-C₁₀-alkylene” (which may also be referred to as aralkyl) as used herein refers to C₆-C₁₀-aryl-substituted C₁-C₁₀-alkyl radicals having at least one unsubstituted or substituted C₆-C₁₀-aryl group, as defined herein. The alkyl group of the aralkyl radical may be interrupted by one or more nonadjacent groups selected from O, S and NR^{b}, wherein R^{b} is as defined above. C₆-C₁₀-aryl-C₁-C₁₀-alkylene is preferably phenyl-C₁-C₁₀-alkylene, more preferably phenyl-C₁-C₄-alkylene, for example benzyl, 1-phenylethyl, 2-phenylethyl, 1-phenylprop-1-yl, 2-phenylprop-1-yl, 3-phenylprop-1-yl, 1-phenylbut-1-yl, 2-phenylbut-1-yl, 3-phenylbut-1-yl, 4-phenylbut-1-yl, 1-phenylbut-2-yl, 2-phenylbut-2-yl, 3-phenylbut-2-yl or 4-phenylbut-2-yl; preferably benzyl and 2-phenylethyl.

The term “hetaryl” or “heteroaryl” as used herein refers to heteroaromatic, monocyclic, bicyclic or tricyclic condensed systems with 5, 6, 7, 8, 9, 10, 11, 12, 13 or 14 ring members in which at least one of the rings is aromatic and which contain 1, 2, 3 or 4 heteroatoms selected from N, S or O. Monocyclic hetaryl groups are preferably 5- or 6-membered hetaryl groups comprising 1, 2, 3 or 4 heteroatoms selected from O, S or N such as 2-furyl (furan-2-yl), 3-furyl (furan-3-yl), 2-thienyl (thiophen-2-yl), 3-thienyl (thio-phen-3-yl), 1H-pyrrol-2-yl, 1H-pyrrol-3-yl, pyrrol-1-yl, imidazol-2-yl, imidazol-1-yl, imid-azol-4-yl, pyrazol-1-yl, pyrazol-3-yl, pyrazol-4-yl, pyrazol-5-yl, 3-isoxazolyl, 4-isoxazolyl, 5-isoxazolyl, 3-isothiazolyl, 4-isothiazolyl, 5-isothiazolyl, 2-oxazolyl, 4-oxazolyl, 5-oxazolyl, 2-thiazolyl, 4-thiazolyl, 5-thiazolyl, 1,2,4-oxadiazol-3-yl, 1,2,4-oxadiazol-5-yl, 1,3,4-oxadiazol-2-yl, 1,2,4-thiadiazol-3-yl, 1,2,4-thiadiazol-5-yl, 1,3,4-thiadiazol-2-yl, 4H-[1,2,4]-triazol-3-yl, 1,3,4-triazol-2-yl, 1,2,3-triazol-1-yl, 1,2,4-triazol-1-yl, pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl. Bicyclic throughout aromatic heteroaryl is 9- or 10-membered and contains 1, 2, 3 or 4 heteroatoms selected from O, S or N. Examples are quinolinyl, isoquinolinyl, indolyl, isoindolyl, indolizinyl, benzofuranyl, isobenzofuranyl, benzothiophenyl, benzoxazolyl, benzisoxazolyl, benzthi-azolyl, benzoxadiazolyl, benzothiadiazolyl, benzoxazinyl, benzopyrazolyl, benzimidaz-olyl, benzotriazolyl, benzotriazinyl.

When # or * appear in a formula showing a substructure of a compound of the present invention, it denotes the attachment bond in the remainder molecule.

With respect to the intended use of the compounds of formula (I), (Ia), (Ib), particular preference is given to the following meanings of the substituents X, R¹ to R⁶ and the variables n and x, in each case on their own or in combination:
R¹ and R², independently of each other, are preferably selected from the group consisting of linear C₁-C₂₄-alkyl, branched C₃-C₂₄-alkyl, C₅-C₈-cycloalkyl, C₆-C₁₀-aryl and C₆-C₁₀-aryl-C₁-C₁₀-alkylene, where the rings of cycloalkyl, aryl and aryl-alkylene in the three last-mentioned radicals are unsubstituted or substituted by 1, 2, 3, 4 or 5 identical or different radicals R^{a}.

More preferably, R¹ and R², independently of each other, are selected from the group consisting of linear C₁-C₂₄-alkyl, branched C₃-C₂₄-alkyl, C₅-C₈-cycloalkyl, phenyl and phenyl-C₁-C₁₀-alkylene, where the rings of cycloalkyl, phenyl and phenyl-alkylene in the three last-mentioned radicals are unsubstituted or substituted by 1, 2 or 3 identical or different substituents R^{a}, wherein R^{a} is as defined above.

Even more preferably, R¹ and R², independently of each other, are selected from the group consisting of C₅-C₈-cycloalkyl that is unsubstituted or substituted by one, two or three C₁-C₆-alkyl substituents; linear C₄-C₂₀-alkyl; a radical of formula (A.1); a radical of formula (A.2); a radical of formula (A.3); a radical of formula (B.1); and a radical of formula (B.2)

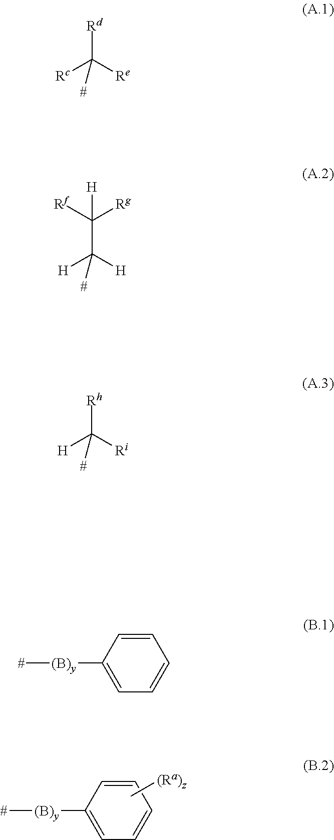

in which
# represents the bonding site to the imide nitrogen atom;
R^{c}, R^{d} and R^{e}, in formula (A.1) are independently selected from C₁-C₁₇-alkyl, where the sum of the carbon atoms of the R^{c}, R^{d} and R^{e} radicals is an integer from 3 to 19;
R^{f} and R^{g}, in formula (A.2) are independently selected from C₁-C₁₇-alkyl, where the sum of the carbon atoms of the R^{f} and R⁹ radicals is an integer from 2 to 18;
R^{h} and Rⁱ, in formula (A.3), independently from each other are selected from C₁-C₁₈-alkyl, where the sum of the carbon atoms of the R^{h} and Rⁱ radicals is an integer from 3 to 19;
B, where present in formulae (B.1) and (B.2), is a C₁-C₁₀-alkylene group;
y is 0 or 1;
each R^{a} is independently selected from the group consisting of C₁-C₂₄-alkyl, C₁-C₂4-fluoroalkyl, C₁-C₂₄-alkoxy, fluorine, chlorine and bromine; preferably C₁-C₂₄-alkyl; more preferably linear C₁-C₁₀-alkyl or branched C₃-C₁₀-alkyl; in particular C₁-C₆-alkyl, e.g. linear C₁-C₆-alkyl or branched C₃-C₆-alkyl; more particularly branched C₃-C₆-alkyl;
z in formula (B.2) is 1, 2 or 3.

Among the radicals of formulae (A.1), (A.2) and (A.3), the radical of formula (A.3) is preferred. In the context of the radical (A.3), R^{h} and Rⁱ, independently of each other, are preferably selected from linear C₄-C₁₀-alkyl.

Among the radicals of formulae (B.1) and (B.2), those are preferred, in which y is 0, i.e. the variable B is absent. Irrespectively of its occurrence, in the context of the radicals of formulae (B.1) and (B.2), each R^{a} is preferably C₁-C₂₄-alkyl, more preferably linear C₁-C₁₀-alkyl or branched C₃-C₁₀-alkyl, e.g. linear C₁-C₆-alkyl or branched C₃-C₆-alkyl; in particular branched C₃-C₆-alkyl; and especially isopropyl or tert-butyl. In particular, the radical of formula (B.2) is preferred. Specific examples of radicals of formula (B.2) are 2,6-dimethylphenyl, 2,4-di(tert-butyl)phenyl, 2,6-diisopropylphenyl or 2,6-di(tert-butyl)phenyl.

Most preferably, R¹ and R² have the same meaning and are linear C₄-C₂₀-alkyl, a radical of formula (A.1), a radical of formula (A.2), a radical of formula (A.3) or a radical of formula (B.2), in which y is 0, i.e. the variable B is absent.

A specific embodiment of the present invention relates to compounds of formula (I), wherein the radicals R¹ and R² are each a radical of formula (B.2), in which y is 0, i.e. the variable B is absent; R^{a} is linear C₁-C₁₀-alkyl or branched C₃-C₁₀-alkyl, in particular linear C₁-C₆-alkyl or branched C₃-C₆-alkyl, specially branched C₃-C₆-alkyl; and z is 1, 2 or 3, especially 1 or 2.

Preferred compounds according to the invention are compounds of formula (I), wherein x is 0.

Preferred compounds according to the invention are also compounds of formula (I), wherein x is 1 or 2. According to a preferred embodiment, each X is independently phenoxy, which is substituted by one substituent R⁷ and one or two substituents R^{7a}, wherein R⁷ and R^{7a} have one of the above general, or, in particular, one of the below preferred meanings. In case of two substituents R^{7a}, the substituents R^{7a} may be identical or different, but are preferably the same. Preferably, R⁷ is attached to phenoxy in para-position, relative to the attachment point of X to the terrylene or quaterrylene scaffold; and at least one of the substituents R^{7a} is attached to phenoxy in the ortho-position. In case of two substituents R^{7a}, preferably both R^{7a} are attached to phenoxy at both ortho-positions. Preferably, R⁷ is linear or branched C₆-C₁₅-alkyl and more preferably linear or branched C₆-C₁₂-alkyl. Specifically, R⁷ is branched C₆-C₁₂-alkyl. Preferably, each R^{7a} is independently linear C₁-C₁₀-alkyl or branched C₃-C₁₀-alkyl. Specifically R^{7a} is branched C₃-C₁₀-alkyl.

Likewise preferably, each X is independently phenyl which is substituted by one substituent R⁸ and zero, one or two substituents R^{8a}, wherein R⁸ and R^{8a} have one of the above general, or, in particular, one of the below preferred meanings. Preferably, phenyl is substituted by R⁸ in the para-position, relative to the attachment point of X to the terrylene or quaterrylene scaffold. Preferably, R⁸ is linear or branched C₅-C₁₂-alkyl, in particular linear C₅-C₁₂-alkyl. Preferably each R^{8a}, if present, is C₁-C₁₂-alkyl. Spe-cially, phenyl is not substituted by R^{8a}.

If x is 2, both X preferably have the same meaning.

Preferred compounds according to the invention are compounds of formula (I) wherein R³, R⁴, R⁵ and R⁶, independently of each other, are selected from phenoxy, which is substituted by one substituent R⁷, and by one or two substituents R^{7a}, wherein R⁷ and R^{7a} have one of the above general, or, in particular, one of the below preferred meanings. In case of two substituents R^{7a}, the substituents R^{7a} may be identical or different, but are preferably the same. Preferably, R⁷ is attached to phenoxy in para-position, relative to the attachment point of R³ to R⁶ to the terrylene or quaterrylene scaffold; and at least one of the substituents R^{7a} is attached to phenoxy in the ortho-position. In case of two substituents R^{7a}, preferably both R^{7a} are attached to phenoxy at both ortho-positions. Preferably, R⁷ is linear or branched C₆-C₁₅-alkyl and more preferably linear or branched C₆-C₁₂-alkyl. Specifically R⁷ is branched C₆-C₁₂-alkyl. Preferably, each R^{7a} is independently linear C₁-C₁₀-alkyl or branched C₃-C₁₀-alkyl. Specifically R^{7a} is branched C₃-C₁₀-alkyl.

Likewise preferred compounds according to the invention are compounds of formula (I) wherein R³, R⁴, R⁵ and R⁶, independently of each other, are phenyl which is substituted by one substituent R⁸ and zero, one or two substituents R^{8a}, wherein R⁸ and R^{8a} have one of the above general, or, in particular, one of the below preferred meanings. Preferably, phenyl is substituted by R⁸ in the para-position. Preferably, R⁸ is linear or branched C₅-C₁₂-alkyl. Preferably each R^{8a}, if present, is C₁-C₁₂-alkyl. Spe-cially, phenyl is not substituted by R^{8a}.

Preferably, R⁷ is C₆-C₁₅-alkyl, more preferably linear C₆-C₁₂-alkyl or branched C₆-C₁₂-alkyl. Examples for linear C₆-C₁₂-alkyl are n-hexyl, n-heptyl, n-octyl, n-nonyl, n-decyl, n-undecyl or n-dodecyl. Examples for branched C₆-C₁₂-alkyl are 1,1-dimethylbutyl, 2,2-dimethylbutyl, 1-methylpentyl, 2-methylpentyl, 1-methylhexyl, 1-ethylpentyl, 1-methylheptyl, 2-ethylhexyl, 6-methylheptyl (isooctyl), 1,1,3,3-tetramethylbutyl (tert-octyl), isononyl, 1,1-dimethylheptyl, isodecyl and positional isomers thereof. Specifically, R⁷ is branched C₆-C₁₂-alkyl. In particular, R⁷ is tert-octyl.

Preferably, R^{7a} is linear C₁-C₁₀-alkyl or branched C₃-C₁₀-alkyl. Examples for linear C₁-C₁₀-alkyl are methyl, ethyl, n-propyl, n-butyl, n-pentyl, n-hexyl, n-heptyl, n-octyl, n-nonyl and n-decyl. Examples for branched C₃-C₁₀-alkyl are isopropyl, sec-butyl, iso-butyl, tert-butyl, 1-methylbutyl, 2-methylbutyl, 3-methylbutyl, 2,2-dimethylpropyl, 1-ethylpropyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpro-pyl, 2-ethylhexyl, 1,1,3,3-tetramethylbutyl (tert-octyl), isononyl, 1,1-dimethylheptyl, isodecyl and positional isomers thereof. More preferably, R^{7a} is branched C₃-C₁₀-alkyl.

R⁸ is preferably linear or branched C₅-C₁₂-alkyl. Examples of linear C₅-C₁₂-alkyl are n-pentyl, n-hexyl, n-heptyl, n-octyl, n-nonyl, n-decyl, n-undecyl or n-dodecyl. Examples for branched C₅-C₁₂-alkyl are isopentyl, neopentyl, tert-pentyl, 1,1-dimethylbutyl, 2,2-dimethylbutyl, 1-methylpentyl, 2-methylpentyl, 1-methylhexyl, 1-ethylpentyl, 1-methylheptyl, 2-ethylhexyl, 6-methylheptyl (isooctyl), 1,1,3,3-tetramethylbutyl (tert-octyl), isononyl, 1,1-dimethylheptyl, isodecyl and the position isomers thereof.

R^{8a}, if present, is preferably C₁-C₁₂-alkyl. Specifically, R^{8a} is absent.

Preference is given to compounds of formula (I), wherein n is 1. These com-pounds are also referred to as terrylene compounds of formula (I).

Likewise preference is given to compounds of formula (I), wherein n is 2. These compounds are also referred to as quaterrylene compounds of formula (I).

Even more preferably, in the compounds of formula (I) the substituents and indi-ces are defined as follows:
n is 1 or 2;
x is 0 when n is 1;
x is 1 or 2 when n is 2;
R¹ and R² are identical and are C₄-C₂₀-alkyl, C₅-C₈-cycloalkyl or phenyl, wherein the two last-mentioned substituents are substituted by one, two or three C₁-C₆-alkyl substituents;
X, R³, R⁴, R⁵ and R⁶, independently of each other are, are phenoxy, which is substi-tuted by one substituent R⁷, and by one or two substituents R^{7a}; wherein preferably R⁷ is attached to phenoxy in para-position and at least one of the substituents R^{7a} is attached to phenoxy in the ortho-position; or are phenyl which is substituted by one substituent R⁸ and zero, one or two substituents R^{8a}, wherein preferably phenyl is substituted by R⁸ in the para-position.
R⁷ is linear or branched C₆-C₅-alkyl;
R^{7a} is linear C₁-C₁₀-alkyl or branched C₃-C₁₀-alkyl;
R⁸ is C₅-C₁₂-alkyl; and
R^{8a}, if present, is C₁-C₁₂-alkyl.

In a particular embodiment, the compound of formula (1) is a compound of formula (1) as defined above, wherein
n is 1 or 2;
x is 0, when n is 1;
x is 1 or 2 when n is 2;
R¹ and R² are each phenyl, which is substituted by one, two or three C₁-C₆-alkyl substituents;
X, R³, R⁴, R⁵ and R⁶, independently of each other, are phenoxy, which is substituted by one substituent R⁷ and one or two substituents R^{7a}; wherein preferably R⁷ is attached to phenoxy in para-position and at least one of the substituents R^{7a} is attached to phenoxy in the ortho-position; or phenyl, which is substituted by one substituent R⁸ and zero, one or two substituents R^{8a};
R⁷ is linear or branched C₆-C₁₂-alkyl; specially branched C₆-C₁₂-alkyl;
R^{7a} is linear C₁-C₁₀-alkyl or branched C₃-C₁₀-alkyl;
R⁸ is linear or branched C₅-C₁₂-alkyl;
R^{8a}, if present, is C₁-C₁₂-alkyl.

More particularly, the compound of formula (1) is a compound of formula (1) as de-fined above, wherein
n is 1 or 2;
x is 0, when n is 1;
x is 1 or 2 when n is 2;
R¹ and R² are each phenyl, which is substituted by one, two or three branched C₃-C₆-alkyl substituents;
R³, R⁴, R⁵, R⁶ and X, if present, have the same meaning and are phenoxy, which is substituted by one substituent R⁷ and one or two substituents R^{7a}; wherein R⁷ is attached to phenoxy in para-position and the one or two substituents R^{7a} are attached to phenoxy in the ortho-position; or phenyl, which is substituted by one substituent R⁸ and zero substituents R^{8a};
R⁷ is branched C₆-C₁₂-alkyl;
R^{7a} is branched C₃-C₁₀-alkyl;
R⁸ is linear C₅-C₁₂-alkyl.

According to a preferred embodiment of the invention, if n is 1, the radicals R³, R⁴, R⁵ and R⁶ have the same meaning and X is absent.

According to a preferred embodiment of the invention, if n is 2, the radicals R³, R⁴, R⁵, R⁶ and X have the same meaning.

Preferred compounds of the formula (1) are especially those of the formula (Ia)

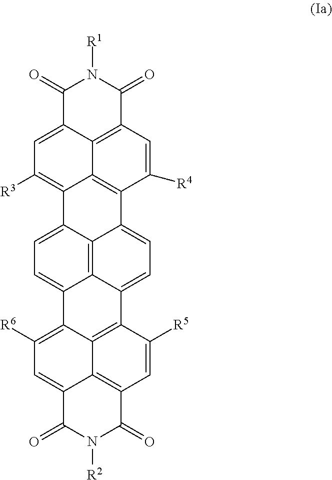

wherein
R¹ and R² have the same meaning and are selected from the group consisting of C₄-C₂₀-alkyl, C₅-C₈-cycloalkyl and phenyl, wherein the two last-mentioned substit-uents are substituted by one, two or three C₁-C₆-alkyl substituents; and
R³, R⁴, R⁵ and R⁶ have the same meaning and are selected from the group consisting of
   phenoxy, which is substituted by one substituent R⁷ and one or two substituents R^{7a}; where specifically phenoxy is substituted by R⁷ in the para-position and by one or two substituents R^{7a}; and where more specifically phenoxy is substituted by R⁷ in the para-position and at least one of the substituents R^{7a} is attached to phenoxy in the ortho-position; or
   phenyl which is substituted by one R⁸ substituent; where specifically phenyl is substituted by R⁸ in the para-position;
wherein
R⁷, R^{7a} and R⁸ have one of the above general or, in particular, one of the above preferred meanings.

In a particular embodiment, the compound of formula (1) is a compound of formula (Ia) as defined above, wherein
R¹ and R² are each phenyl, which is substituted by one, two or three C₁-C₆-alkyl substituents;
R³, R⁴, R⁵ and R⁶ are phenoxy, which is substituted by one substituent R⁷ and one or two substituents R^{7a}, wherein preferably R⁷ is attached to phenoxy in para-position and at least one of the substituents R^{7a} is attached to phenoxy in the ortho-position;
R⁷ is linear or branched C₆-C₁₂-alkyl; specially branched C₆-C₁₂-alkyl; and
R^{7a} is linear C₁-C₁₀-alkyl or branched C₃-C₁₀-alkyl; specially branched C₃-C₁₀-alkyl.

More particularly, the compound of formula (1) is a compound of formula (Ia) as defined above, wherein
R¹ and R² are each phenyl, which is substituted by one, two or three branched C₃-C₆-alkyl substituents;
R³, R⁴, R⁵ and R⁶ have the same meaning and are phenoxy, which is substituted by one substituent R⁷ and one or two substituents R^{7a}, wherein R⁷ is attached to phenoxy in para-position and the one or two substituents R^{7a} are attached to phenoxy in the ortho-position;
R⁷ is branched C₆-C₁₂-alkyl; and
R^{7a} is branched C₃-C₁₀-alkyl.

Likewise in a particular embodiment, the compound of formula (1) is a compound of formula (Ia) as defined above, wherein
R¹ and R² are each phenyl, which is substituted by one, two or three C₁-C₆-alkyl substituents;
R³, R⁴, R⁵ and R⁶ are phenyl, which is substituted by one substituent R⁸; and
R⁸ is linear or branched C₅-C₁₂-alkyl, specially linear C₅-C₁₂-alkyl.

More particularly, the compound of formula (1) is a compound of formula (Ia) as defined above, wherein
R¹ and R² are each phenyl, which is substituted by one, two or three branched C₃-C₆-alkyl substituents;
R³, R⁴, R⁵ and R⁶ are phenyl, which is substituted by one substituent R⁸; and
R⁸ is linear C₅-C₁₂-alkyl.

In the compounds of formula (Ia), R¹, R², R³, R⁴, R⁵ and R⁶ have one of the above preferred meanings. Specifically, in the compounds of formula (Ia), R¹ and R² are 2,6-dimethylphenyl, 2,4-di(tert-butyl)phenyl, 2,6-diisopropylphenyl or 2,6-di(tert-bu-tyl)phenyl. Specifically, in the compounds of formula (Ia), R³, R⁴, R⁵ and R⁶ preferably are each 2,6-diisopropyl-4-(1,1,3,3-tetramethylbutyl)-phenoxy, 2,6-di-(tert-butyl)-4-(1,1,3,3-tetramethylbutyl)-phenoxy, 2,4-bis(1,1,3,3-tetramethylbutyl)phenoxy or 2,4,6-tris(1,1,3,3-tetramethylbutyl)phenoxy. Likewise preferably, in the compounds of formula (Ia), R³, R⁴, R⁵ and R⁶ are each 4-n-hexylphenyl or 4-n-octylphenyl.

Preferred compounds of the formula (1) are also those of the formula (Ib)

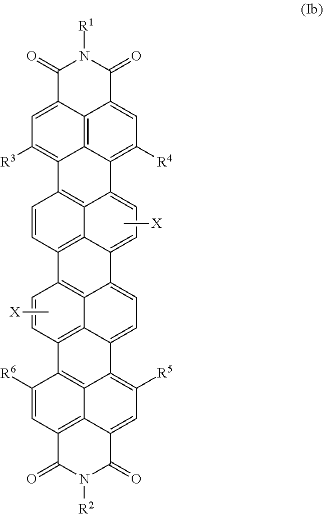

wherein
R¹ and R² have the same meaning and are selected from C₄-C₂₀-alkyl, C₅-C₈-cycloalkyl or phenyl, wherein the two last-mentioned substituents are substituted by one, two or three C₁-C₆-alkyl substituents;
R³, R⁴, R⁵ and R⁶ have the same meaning and are phenoxy, which is substituted by one substituent R⁷ and one or two substituents R^{7a}; where specifically phenoxy is substituted in the para-position by R⁷ and by one or two substituents R^{7a}; and where more specifically, phenoxy is substituted in the para-position by R⁷ and in the ortho-position(s) by the one or two substituents R^{7a}; and
each X is independently phenoxy, which is substituted by one substituent R⁷ and one or two substituents R^{7a}; where specifically phenoxy is substituted in the para-position by R⁷ and by one or two substituents R^{7a}; and where more specifically, phenoxy is substituted in the para-position by R⁷ and in the ortho-position(s) by the one or two substituent(s) R^{7a};
   wherein
   R⁷ and R^{7a} have one of the above general or, in particular, one of the above preferred meanings.

In a particular embodiment, the compound of formula (1) is a compound of formula (Ib) as defined above, wherein
R¹ and R² are each phenyl, which is substituted by one, two or three C₁-C₆-alkyl substituents;
R³, R⁴, R⁵ and R⁶ are phenoxy, which is substituted by one substituent R⁷ and one or two substituents R^{7a}; wherein preferably R⁷ is attached to phenoxy in para-position and at least one of the substituents R^{7a} is attached to phenoxy in the ortho-position;
each X is independently phenoxy, which is substituted by one substituent R⁷ and one or two substituents R^{7a}; wherein preferably R⁷ is attached to phenoxy in para-position and at least one of the substituents R^{7a} is attached to phenoxy in the ortho-position;
R⁷ is linear or branched C₆-C₁₂-alkyl; specially branched C₆-C₁₂-alkyl; and
R^{7a} is linear C₁-C₁₀-alkyl or branched C₃-C₁₀-alkyl; specially branched C₃-C₁₀-alkyl.

In the compounds of formula (Ib), R¹, R², R³, R⁴, R⁵ and R⁶ have one of the above preferred meanings.

More particularly, the compound of formula (I) is a compound of formula (Ib) as defined above, wherein
R¹ and R² are each phenyl, which is substituted by one, two or three branched C₃-C₆-alkyl substituents;
R³, R⁴, R⁵, R⁶ and X have the same meaning and are phenoxy, which is substituted by one substituent R⁷ and one or two substituents R^{7a}; wherein R⁷ is attached to phenoxy in para-position and the one or two substituents R^{7a} are attached to phenoxy in the ortho-position;
R⁷ is branched C₆-C₁₂-alkyl; and
R^{7a} is branched C₃-C₁₀-alkyl.

Specifically, in the compounds of formula (Ib), R¹ and R² are 2,6-dimethylphenyl, 2,4-di-(tert-butyl)-phenyl, 2,6-diisopropylphenyl or 2,6-di-(tert-butyl)-phenyl. Specifically, in the compounds of formula (Ib), X, R³, R⁴, R⁵ and R⁶ have the same meaning and preferably are each 2,6-diisopropyl-4-(1,1,3,3-tetramethylbutyl)-phenoxy, 2,6-di-(tert-butyl)-4-(1,1,3,3-tetramethylbutyl)-phenoxy, 2,4-bis(1,1,3,3-tetramethylbutyl)phenoxy or 2,4,6-tris(1,1,3,3-tetramethylbutyl)phenoxy.

Examples of preferred compounds are

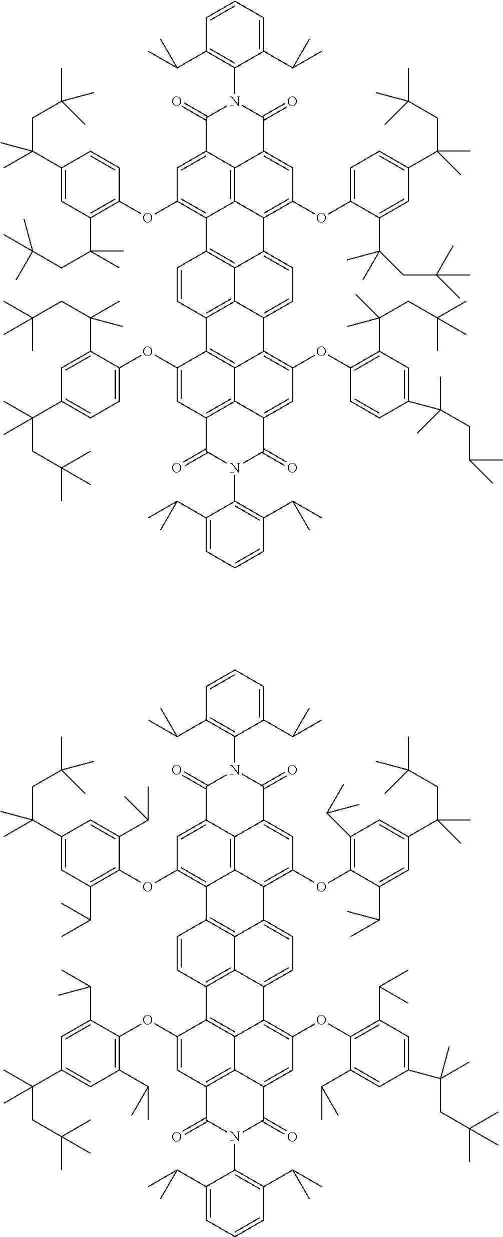

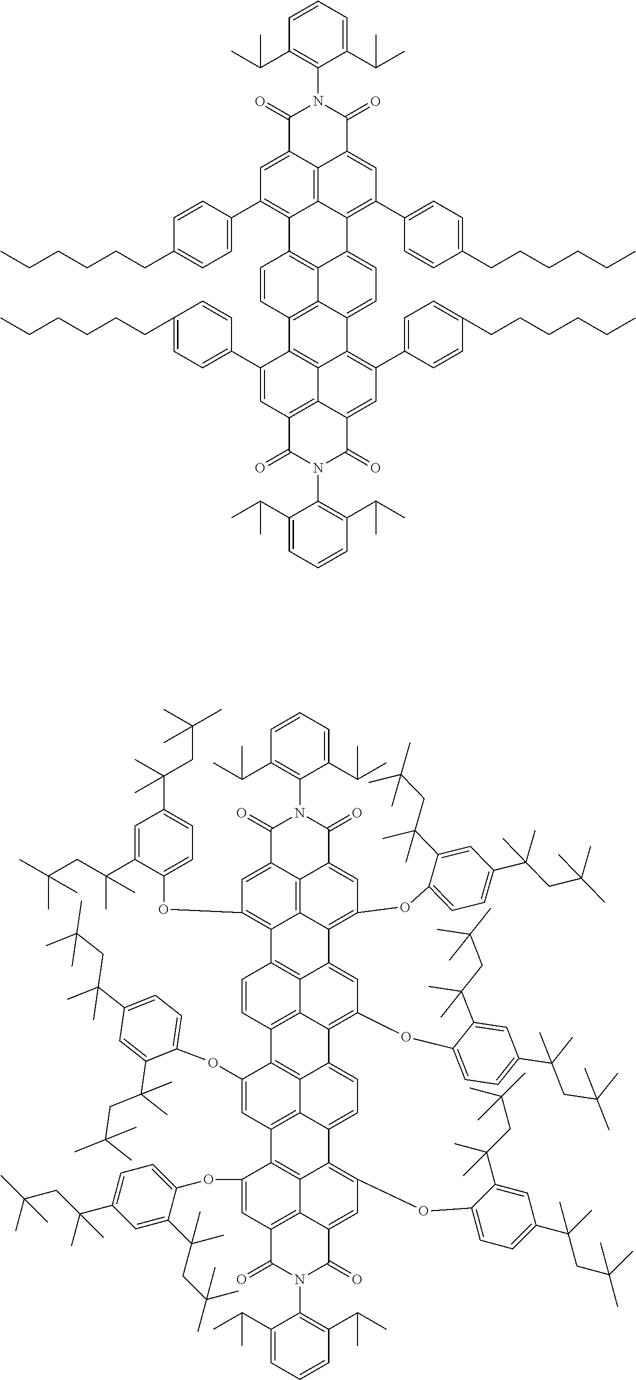

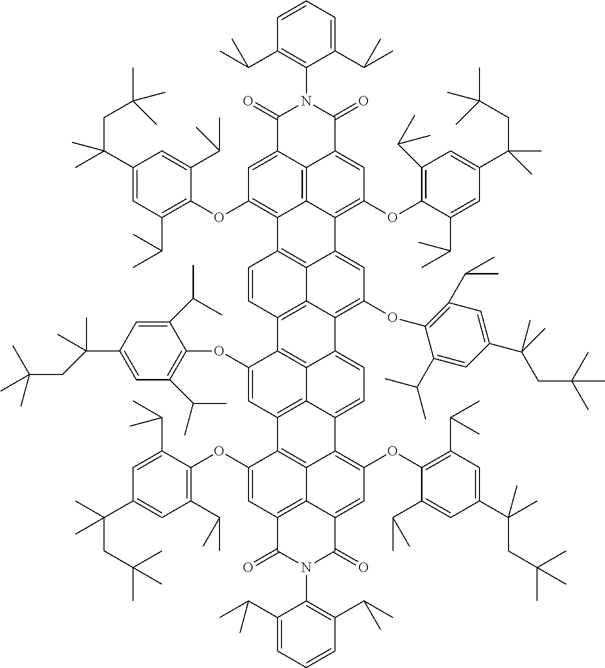 [SMILES: CC(C)c1cc(C(C)(C)CC(C)(C)C)cc(C(C)C)c1Oc1cc2c3c(cc(Oc4c(C(C)C)cc(C(C)(C)CC(C)(C)C)cc4C(C)C)c4c5cc(Oc6c(C(C)C)cc(C(C)(C)CC(C)(C)C)cc6C(C)C)c6c7ccc8c9c(Oc%10c(C(C)C)cc(C(C)(C)CC(C)(C)C)cc%10C(C)C)cc%10c%11c(cc(Oc%12c(C(C)C)cc(C(C)(C)CC(C)(C)C)cc%12C(C)C)c(c%12cc(Oc%13c(C(C)C)cc(C(C)(C)CC(C)(C)C)cc%13C(C)C)c(c%13ccc(c1c34)c5c%136)c7c8%12)c%119)C(=O)N(c1c(C(C)C)cccc1C(C)C)C%10=O)C(=O)N(c1c(C(C)C)cccc1C(C)C)C2=O]

The compounds of the present invention can be prepared by using routine techniques familiar to a skilled person. In particular, the compounds of the formula (1) can be prepared according to the following routes or as described in the experimental part of this application. A skilled person will readily understand that if x=0, the radical X is absent in the compound of formula (1).

Compounds of formula (1), wherein R³, R⁴, R⁵, R⁶ and X, if present, are C₆-C₁₀-aryloxy which is substituted by one substituent R⁷ and one or more identical or different substituents R^{7a} can be prepared in analogy to standard methods as described in WO 03/104232 or WO 2007/006717.

For example, they can be prepared by reacting a halogenated compound of formula (XVII)

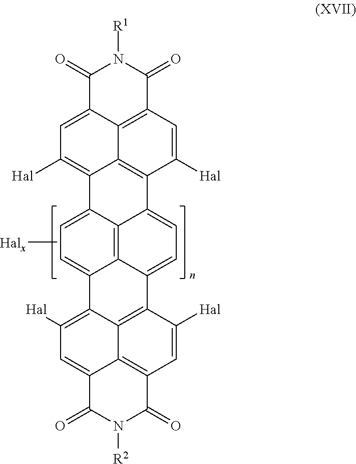

wherein
n, R¹ and R² are as defined above; Hal is a halogen atom, preferably chlorine or bromine,
x is 0, 1 or 2;
with a hydroxy-containing aromatic compounds of formula (XVIII)

Ar—OH (XVIII)
wherein
Ar is C₆-C₁₀-aryl, which is substituted by one substituent R⁷ and one or more identical or different substituents R^{7a}, wherein R⁷ and R^{7a} are as defined above; to obtain a compound of formula (1), wherein R³, R⁴, R⁵, R⁶ and X are C₆-C₁₀-aryloxy which is substituted by one substituent R⁷ and one or more identical or different substituents R^{7a}.

The reaction is typically carried out in the presence of a non-nucleophilic solvent such as a polar aprotic solvent or a nonpolar aprotic solvent. Suitable polar aprotic solvents are especially aliphatic carboximides such as N,N-di-C₁-C₄-alkyl-C₁-C₄-carboximides and lactams such as dimethylformamide, diethylformamide, dimethylacetamide, dimethylbutyramide and N-methylpyrrolidone (NMP). Suitable nonpolar aprotic solvents include aromatic hydrocarbons. Preferably, the reaction is carried out in the presence of a polar aprotic solvent. It is also possible to use solvent mixtures.

The reaction is typically carried out in the presence of a base. Suitable bases are in particular inorganic and organic alkali metal or alkaline earth metal based, the alkali metal bases being particularly preferred. Examples of inorganic bases are the carbonates and hydrogencarbonates, hydroxides, hydrides and amides of alkali metals and alkaline earth meats; examples of organic bases are alkoxides, (phenyl) alkylamides and triphenylmethylmetallates based on alkali metals and alkaline earth metals. Preferred bases are the carbonates and hydrogencarbonates. Preferred alkali metals are lithium, sodium, potassium and cesium. Very particularly preferred are sodium carbonate, potassium carbonate and cesium carbonate

The reaction may be carried out in the presence of a transfer catalyst such as quaternary ammonium or phosphonium salt.

The reaction temperature is generally within the range from 30 to 150° C.

It may be advantageous to initially charge only a portion of the compound of formula (XVIII) and of the base and to add the rest only at a later stage.

The starting compound of formula (XVII) can be prepared as described in WO 2006/037539 or in analogy to the method described therein.

The starting hydroxy-containing aromatic compounds of formula (XVIII) are either commercially available or can be prepared by alkylating an appropriate substituted hy-droxyl-containing aromatic compound with an olefin in the sense of a Friedel-Crafts-al-kylation.

Compounds of formula (1), wherein R³, R⁴, R⁵, R⁶ and X, if present, are aryl which is substituted by one substituent R⁸ and zero, one or more identical or different substit-uents R^{8a} can be prepared by a cross-coupling reaction in the presence of a transition metal catalyst starting from a compound of formula (XVII) and an organometallic compound of formula (XIX)

Ar*-Met (XIX)
wherein
Ar* is aryl which is substituted by one substituent R⁸ and zero, one or more identical or different substituents R^{8a}; and
Met is B(OH)₂, B(OR′)(OR″), Zn-Hal or Sn(R*)₃,
   in which
   R′ and R″ are each independently hydrogen, C₁-C₃₀-alkyl, C₅-C₈-cycloalkyl, C₆-C₁₄-aryl or heteroaryl, or R′ and R″ together are C₂-C₄-alkylene which optionally bears 1, 2, 3, 4, 5, 6, 7 or 8 substituents selected from C₁-C₄-alkyl, C₅-C₈-cycloalkyl and C₆-C₁₄-aryl; Hal is chlorine or bromine; and
   R* is C₁-C₅-alkyl or phenyl
to obtain a compound of formula (1), in which R³, R⁴, R⁵, R⁶ and X, if present, are aryl which is substituted by one substituent R⁸ and zero, one or more identical or different substituents R^{8a}.

Preference is given to effecting the reaction in the presence of catalytically active amounts of a transition metal of transition group VIII of the Periodic Table (group 10 according to IUPAC), for example nickel, palladium or platinum, especially in the presence of a palladium catalyst. Suitable catalysts are, for example, palladium-phosphine complexes such as tetrakis(triphenylphosphine)palladium(0), PdCl₂(o-tolyl₃P)₂, bis(tri-phenylphosphine)palladium(II) chloride, the [1,1′-bis(diphenylphosphino)ferrocene]pal-ladium(II) chloride-dichloromethane complex, bis[1,2-bis(diphenylphosphino)ethane]-palladium(0) and [1,4-bis(diphenylphosphino)butane]palladium(II) chloride, palladium on activated carbon in the presence of phosphine compounds, and palladium(II) com-pounds such as palladium(II) chloride or bis(acetonitrile)palladium(II) chloride in the presence of phosphine compounds such as triphenylphosphine, 1,1′-bis(diphenyl-phosphino)ferrocene, 1,2-bis(diphenylphosphino)ethane, 1,3-bis(diphenylphosphino)-propane and 1,4-bis(diphenylphosphino)butane. The amount of catalyst is typically 10 to 150 mol %, based on the compounds of the formula (II).

Especially suitable organometallic compounds (XIX) are an appropriately substi-tuted arylboronic acid and arylboronic esters of formula (XIX), where Met=B(OH)₂ or B(OR′)(OR″) where R′, R″═C₁-C₄-alkyl, or R′ and R″ together are C₂-C₄-alkylene optionally bearing 1, 2, 3 or 4 substituents selected from C₁-C₄-alkyl.

The arylboronic acids and esters thereof are known from the literature, commercially available, or can be prepared from the corresponding arylmagnesium compounds by reaction with appropriate boric esters.

The reaction of (XVII) with the organometallic compound (XIX), especially in the case of the Suzuki coupling, is effected under basic conditions. Suitable bases are alkali metal carbonates and alkali metal hydrogencarbonates such as sodium carbonate, potassium carbonate, cesium carbonate, sodium hydrogencarbonate, alkaline earth metal carbonates and alkaline earth metal hydrogencarbonates such as magnesium carbonate or magnesium hydrogencarbonate, or tertiary amines such as triethylamine, trimethylamine, triisopropylamine or N-ethyl-N-diisopropylamine.

Typically, the coupling of the compound of formula (XVII) with the compound of formula (XIX) is effected in a solvent. Suitable solvents are organic solvents such as aromatics, e.g. toluene, mesitylene, acyclic ethers, e.g. 1,2-dimethoxyethane, cyclic ethers such as tetrahydrofuran or 1,4-dioxane, polyalkylene glycols such as diethylene glycol, carbonitriles such as acetonitrile, propionitrile, carboxamides such as dimethylformamide or dimethylacetamide. In the Suzuki coupling, the aforementioned solvents can also be used in a mixture with water; for example, the ratio of organic solvent to water may be in the range from 5:1 to 1:5.

At least one mole of the organometallic compound (XIX) is used per mole of halogen atom to be exchanged. It may be advantageous to use a 5 to 30% molar excess of organometallic compound of formula (XIX) per mole of halogen atom to be exchanged.

Compounds of the formula (XIX) are commercially available or can be prepared according to known methods in the art.

All reactions are typically carried out in the absence of oxygen and moisture.

As a rule, the reaction mixtures are worked up in the customary manner, for example by mixing with water, separating the phases, and, if appropriate purifying the crude product by chromatography. If the end product is obtained as a solid, it may be purified by recrystallization.

The compounds of formula (I) according to the invention are notable for their high solubility or dispersibility in, i.e. their very good compatibility with, not only polar media (e.g. aliphatic alcohols and esters on the one hand and polyacrylates, polycarbonates and polyesters on the other) but also nonpolar media (such as alkanes and polyolefins, for example). Accordingly, the compounds of the formula (I) according to the invention may be incorporated without any problem into organic and inorganic materials and are therefore suitable for a whole series of end uses, some of which will be listed by way of example below.

In general, the terrylene compounds of formula (I) are fluorescent dyes or pigments that absorb light having a wavelength in the range from 450 to 800 nm. They generally have their absorption maximum in the range from 690 to 750 nm. They generally emit light in a range from 700 to 950 nm. The fluorescence light thusly generated is advantageously detected with a semiconductor detector, especially with a silicon photodiode or a germanium photodiode. For these applications, it is important to use the compound of formula (I) in high concentration to convert as much of the absorbed light as possible.

In general, the fluorescent or non-fluorescent quaterrylene compounds of formula (I) are useful as pigments or fluorescent dyes. They absorb in a wavelength range from 700 to 1000 nm and generally have their absorption maximum in the range from 700 to 950 nm. The fluorescence is typically in the near-infrared region.

In sum, the compounds of formula (1) are characterized by intense absorption in the wavelength range from 690 to 1000 nm, namely in the long-wavelength red and near infrared region of the electromagnetic spectrum, high heat stability, high light stability and low manufacture costs. The compounds of formula (1) are also characterized by a high solubility or dispersibility in liquid media such as printing inks or fuel oils. Spe-cially, the compounds of formula (1) are also characterized by high fastness to boiling water and high fastness to acids and alkalines. Specially, the compounds of formula (1) are also characterized by high compatibility with a number of formulations, in particular printing ink formulations used especially in security printing or 3D printing. Compositions such as inks comprising a compound of formula (1) are also characterized by good adhesion to paper and other materials such as plastics. Some of the compounds of formula (1) are also characterized by zero or low absorption in the visible spectral range from 380 to less than 750 nm, e.g. they have a transmittance of at least 75% in the range of from 380 to less than 750 nm. Some of the compounds of formula (1) are also characterized by intense emission in the wavelength range from 690 to 1000 nm, i.e., by high fluorescence quantum yield.

Accordingly, the compounds of formula (1) according to the invention are suitable for a wide variety of applications. Thus, the invention further relates to the use of the compound of formula (1) as defined above in security printing, for coloring coatings, in printing inks including printing inks for 3D printing and plastics, for data storage, for optical labels, for security labels in documents and for brand protection, for solar collectors, for optical waveguides, for the laser welding of plastics or as a fluorescent label for biomolecules.

The compounds of the formula (1) according to the present invention are useful as fluorescent dye or pigment. Among these, preference is given to those of the formula (Ia). Specially, the compounds of formula (Ia) are particularly suitable for optical waveguides. In general, the content of compounds of the general formula (Ia) is a total of between 0.0001 to 2% by weight based on the material used for wavelength conversion.

The compounds of formula (1) are also particularly suitable for laser welding of materials, wherein the materials are first contacted with compounds of the general formula (1). In general, the content of compounds of the general formula (1) is a total of between 0.0001 to 1% by weight based on the material to be welded.

The compounds of formula (1) are also particularly suitable in a fluorescent solar concentrator.

The compounds of formula (1) are also particularly suitable for homogeneously coloring high molecular weight organic and inorganic materials, for example, plastics, in particular thermoplastics, coatings and printing inks including printing inks for security printing and printing inks for 3D printing, and also oxidic layer systems.

The compounds of formula (1) are also particularly suitable as near infrared absorbers for heat management and as NIR laser beam-absorbent materials in the fusion treatment of plastics parts. These applications are described in detail, for example, in DE 10 2004 018 547, WO 02/77081 and WO 04/05427.

The compounds of formula (1) are also particularly suitable for laser marking and laser inscription. In this case, the laser light absorbed by the compound of formula (1) brings about heating of the plastic, which leads to its foaming or the conversion of a dye additionally present, and in this way gives rise to a marking or inscription. In addition, the quaterrylene compounds of formula (1) which absorb in the NIR can function in particular as protective filters for NIR radiation.

The compounds of formula (1) are also particularly suitable as labeling groups in detection methods, especially in diagnostic and analytical methods on biological samples, including living cells.

The compounds of formula (1) are also particularly suitable for use in an ink for machine readability and/or security applications. The essential component of the ink is at least one compound of formula (1) that absorbs radiation in the wavelength range of 690-1000 nm. Specifically, the ink also shows a high transparency for visible light.

The compounds of formula (1), owing to their pronounced high molar extinction coefficient, are also of interest for obtaining markings and inscriptions which are either visible to the human eye, when the compound of formula (1) absorbs visible light, or are invisible to the human eye, when the compound of formula (1) is an IR absorber. Thus, the present invention also relates to the use of the compound of formula (1) as defined above for brand protection or as marker for liquids. Useful liquids which can be marked with the compounds of the formula (1) preferably include oils such as fuel oils of mineral or regenerative origin, vegetable and animal fatty oils, and ethereal oils.

Examples of such oils are natural oils such as olive oil, soybean oil or sunflower oil, or natural or synthetic motor oils, hydraulic oils or transmission oils, for example motor vehicle oil or sewing machine oil, or brake fluids and fuel oils of mineral or regenerative origin, in particular fuel oil of mineral origin such as gasoline, kerosene, diesel oil and also heating oil. Particular preference is given to mineral oils such as gasoline, kerosene, diesel oil or heating oil, in particular gasoline, diesel oil or heating oil. Particularly advantageously, the above-mentioned compounds of the formula (1) are used as markers for fuel oils in which labelling is required, for example for tax reasons. In order to minimize the costs of labelling, but also in order to minimize possible interactions of the marked mineral oils with any other ingredients present, such as polyisobutene-amine (PIBA), efforts are made to minimize the amount of markers. A further reason to minimize the amount of markers may be to prevent their possible harmful influences, for example on the fuel intake and exhaust gas outlet region of internal combustion en-gines.

The compound of formula (1) to be used as markers is added to the liquids in such amounts that reliable detection is ensured. Typically, the (weight-based) total content of markers in the marked liquid is from about 0.1 to 5000 ppb, preferably from 1 to 2000 ppb and more preferably from 1 to 1000 ppb. The detection of the marking can be undertaken, for example, with the aid of an (absorption) spectrometer

The compound of the formula (1) may if appropriate also be used in a mixture with other markers/colorants.

To mark the liquids, the compounds are generally added in the form of solutions (stock solutions). Especially in the case of fuel oils, suitable solvents for providing these stock solutions are preferably aromatic hydrocarbons such as toluene, xylene or rela-tively high-boiling aromatics mixtures.

The present invention further relates to an article comprising at least one film layer, the film layer comprising in a polymeric matrix the compound of formula (1) as de-fined above. The compounds of the general formula (1) can be processed into virtually all plastics with the aid of processes known to those skilled in the art.

Preferably, the polymeric matrix comprises at least one thermoplastic polymer. Preferably, the thermoplastic polymer is selected from the group consisting of
homo- and copolymers which comprise at least one copolymerized monomer selected from C₂-C₁₀-monoolefins, 1,3-butadiene, 2-chloro-1,3-butadiene, vinyl alcohol and its C₂-C₁₀-alkyl esters, vinyl chloride, vinylidene chloride, glycidyl acrylate, glycidyl methacrylate, acrylates and methacrylates of C₁-C₁₀-alcohols, vinylaromatics, (meth) acrylonitrile, maleic anhydride, and ethylenically unsaturated mono- and dicarboxylic acids,
homo- and copolymers of vinyl acetals;
polyvinyl esters;
polyvinylchlorides
polycarbonates;
polyesters;
polyethers;
polyether ketones;
thermoplastic polyurethanes;
polysulfides;
polysulfones;
polyether sulfones;
cellulose alkyl esters;
polypropylenes;
polyethylene terephthalates;
and mixtures of two or more thereof.

Likewise possible is incorporation into blends which comprise the above-mentioned plastics as components, or into polymers which have been derived from these classes and have been modified by subsequent reactions. These materials are known and commercially available in a wide variety.

Mention may be made by way of example of polyacrylates having identical or different alcohol moieties derived from the group of the C₄-C₈-alcohols (particularly of bu-tanol, hexanol, octanol, and 2-ethylhexanol), polycarbonate, polymethyl methacrylate (PMMA), methyl methacrylate, butyl acrylate copolymers, acrylonitrile-butadiene-styrene copolymers (ABSs), ethylene-propylene copolymers, ethylene-propylene-diene copolymers (EPDMs), polystyrene (PS), styrene-acrylonitrile copolymers (SANs), acry-lonitrile-styrene-acrylate (ASA), styrene-butadiene-methyl methacrylate copolymers (SBMMAs), styrene-maleic anhydride copolymers, styrene-methacrylic acid copolymers (SMAs), polyoxymethylene (POM), polyvinyl alcohol (PVAL), polyvinyl acetate (PVA), polyvinylbutyral (PVB), polycaprolactone (PCL), polyhydroxybutyric acid (PHB), polyhydroxyvaleric acid (PHV), polylactic acid (PLA), ethylcellulose (EC), cellulose ace-tate (CA), cellulose propionate (CP), and cellulose acetate/butyrate (CAB).

Preferably, the polymeric matrix material comprises, in particular consists of, a polymer selected from the group consisting of polyester, especially polyethylene terephthalate, polycarbonate (PC), polystyrene (PS), polymethyl methacrylate (PMMA), polyvinylchloride, polyamide, polyethylene, polypropylene, styrene/acrylonitrile (SAN), acrylonitrile/butadiene/styrene (ABS) and mixtures of two or more thereof. Particular preference is given to polycarbonate, polyester, especially polyethylene terephthalate, or poly(methyl-methacrylate).

The polymeric matrix material may further comprise suitable stabilizers to stabi-lize the polymer. Such stabilizers are known to the skilled person and include antioxidants, UV absorbers, light stabilizers, hindered amine light stabilizers, antiozonants and the like, in particular hindered amine light stabilizers. The term “hindered amine light stabilizer” refers to sterically hindered amines of the class of compounds typically represented by 2,2,6,6 tetraalkyl piperidines.

In case the polymeric matrix material comprises a stabilizer, the matrix material preferably comprises the stabilizer in an amount of 0.001% by weight to 10% by weight, based on the total weight of the sum of all plastic materials.

According to a further embodiment, the polymeric matrix material comprises at least one scattering agent. Suitable light scattering agents are inorganic white pigments, for example titanium dioxide, barium sulfate, lithopone, zinc oxide, zinc sulfide, calcium carbonate with a mean particle size to DIN 13320 of 0.01 to 10 μm, preferably 0.1 to 1 μm, more preferably 0.15 to 0.4 μm. These light scattering agents are included typically in an amount of 0.01 to 2.0% by weight, preferably 0.05 to 1.0% by weight, more preferably 0.1 to 0.6% by weight, based in each case on the polymer of the layer comprising scattering bodies.

Examples of suitable organic light scattering agents include scattering polymers such as those based on poly(acrylates); poly (alkyl methacrylates), for example poly(methyl methacrylate) (PMMA); poly (tetrafluoroethylene) (PTFE); silicone-based scattering agents, for example hydrolyzed poly(alkyl trialkoxysilanes), and mixtures thereof. The size of these light scattering agents (average diameter-weight average) is usually in the range from 0.5 to 50 μm, preferably 1 to 10 μm. These light scattering agents are typically included in an amount of 1 to 10% by weight, based in each case on the polymer of the layer comprising scattering bodies. Useful light scattering agents are for example a mixture of 3 to 5% by weight of PMMA based scattering agent and 1.5 to 2% by weight of silicone based scattering agent.

Also suitable are light-scattering compositions which contain polymeric particles based on vinyl acrylate with a core/shell morphology in combination with TiO₂ as described in EP-A 634 445.

In a preferred embodiment, the article is a color converter. According to this embodiment, the color converter preferably comprises at least one inorganic white pigment as a scattering body. Likewise preferably, the color converter comprises at least one further fluorescent material selected from garnets, silicates, sulfides, nitrides, oxy-nitrides, quantum dots and organic fluorescent colorants. Among these, preference is given to organic fluorescent colorants B from the groups (B1), (B2), (B3), (B4), (B5), (B6), (B7), (B8), (B9), (B10), (B111), (B12), (B13), (B14), (B15) or mixtures thereof. (B1) a cyanated naphthoylbenzimidazole compound of formula (II)

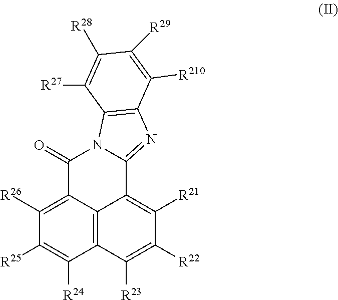

wherein
R²¹, R²², R²³, R²⁴, R²⁵, R²⁶, R²⁷, R²⁸, R²⁹ and R²¹⁰ are each independently hydrogen, cyano or aryl which is unsubstituted or has one or more identical or different substituents R^{2Ar},
   where
   each R^{2Ar} is independently cyano, hydroxyl, mercapto, halogen, C₁-C₂₀-alkoxy, C₁-C₂₀-alkylthio, nitro, —NR^{2Ar2}R^{2Ar3}, —NR^{2Ar2}COR^{2Ar3}, —CONR^{2Ar2}R^{2Ar3}, —SO₂NR^{2Ar2}R^{2Ar3}, —COOR^{2Ar2}, —SO₃R^{2Ar2}, C₁-C₃₀-alkyl, C₂-C₃₀-alkenyl, or C₂-C₃₀-alkynyl, where the three latter radicals are unsubstituted or bear one or more R^{2a} groups, C₃-C₈-cycloalkyl, 3- to 8-membered heterocyclyl, where the two latter radicals are unsubstituted or bear one or more R^{2b} groups, aryl, U-aryl, heteroaryl and U-heteroaryl, where the four latter radicals are unsubstituted or bear one or more R^{2b} groups,
      where
      each R^{2a} is independently cyano, hydroxyl, oxo, mercapto, halogen, C₁-C₂₀-alkoxy, C₁-C₂₀-alkylthio, nitro, —NR^{2Ar2}R^{Ar3}, —NR^{2Ar2}COR^{2Ar3}, —CONR^{2Ar2}R^{Ar3}, —S₂NR^{2Ar2}R^{Ar3}, —COOR^{2Ar2}, —SO₃R^{2Ar2}, C₃-C₈-cycloalkyl, 3- to 8-membered heterocyclyl, aryl or heteroaryl, where the cycloalkyl, heterocyclyl, aryl and heteroaryl radicals are unsubstituted or bear one or more R^{2b} groups;
      each R^{2b} is independently selected from cyano, hydroxyl, oxo, mercapto, halogen, C₁-C₂₀-alkoxy, C₁-C₂₀-alkylthio, nitro, —NR^{2Ar2}R^{2Ar3}, —NR^{2Ar2}COR^{2Ar3}, —CONR^{2Ar2}R^{2Ar3}, —S₂NR^{2Ar2}R^{2Ar3}, —COOR^{2Ar2}, —SO₃R^{2Ar2}, C₁-C₁₈-alkyl, C₂-C₁₈-alkenyl, C₂-C₁₈-alkynyl, C₃-C₈-cycloalkyl, 3- to 8-membered heterocyclyl, aryl and heteroaryl, where the four latter radicals are unsubstituted or bear one or more R^{2b}1 groups,
      each R^{2b}1 is independently selected from cyano, hydroxyl, mercapto, oxo, nitro, halogen, —NR^{2Ar2}R^{2Ar3}, —NR^{2Ar2}COR^{2Ar3}, —CONR^{2Ar2}R^{2Ar3}, —S₂NR^{2Ar2}R^{2Ar3}, —COOR^{2Ar2}, S₃R^{2Ar2}, —SO₃R^{2Ar2}, C₁-C₁₈-alkyl, C₂-C₁₈-alkenyl, C₂-C₁₈-alkynyl, O₁—O₁₂-alkoxy, and C₁-C₁₂-alkylthio,
      U is an —O—, —S—, —NR^{2Ar1}_, —Co—, —SO— or —SO₂— moiety; R^{2Ar1}, R^{2Ar2}, R^{2Ar3} are each independently hydrogen, C_-C₁₈-alkyl, 3- to 8-membered cycloalkyl, 3- to 8-membered heterocyclyl, aryl or heteroaryl, where alkyl is unsubstituted or bears one or more R^{2a} groups, where 3- to 8-membered cycloalkyl, 3- to 8-membered heterocyclyl, aryl and heteroaryl are unsubstituted or bear one or more R^{2b} groups;
   with the proviso that the compound of formula (II) comprises at least one cyano group,
   and mixtures thereof;

### (B2) A Cyanated Perylene Compound of Formula (III)

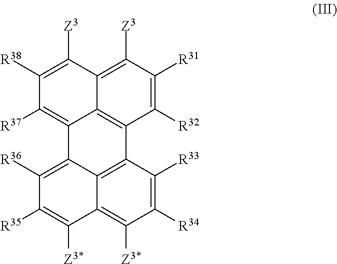

in which
one of the Z³ substituents is cyano and the other Z³ substituent is CO₂R³⁹, CONR³¹⁰R³¹¹, C₁-C₁₈-alkyl, C₂-C₁₈-alkenyl, C₂-C₁₈-alkynyl, C₃-C₁₂-cycloalkyl or C₆-C₁₄-aryl, where C₁-C₁₈-alkyl, C₂-C₁₈-alkenyl, C₂-C₁₈-alkynyl are unsubstituted or bear one or more identical or different Z^{3a} substituents, C₃-C₁₂-cycloalkyl is unsubstituted or bears one or more identical or different Z^{3b} substituents, and C₆-C₁₄-aryl is unsubstituted or bears one or more identical or different Z^{3Ar} substituents;
one of the Z³* substituents is cyano and the other Z³* substituent is CO₂R³⁹, CONR³¹⁰R³¹¹, C₁-C₁₈-alkyl, C₂-C₁₈-alkenyl, C₂-C₁₈-alkynyl, C₃-C₁₂-cycloalkyl or C₆-C₁₄-aryl, where C₁-C₁₈-alkyl, C₂-C₁₈-alkenyl, C₂-C₁₈-alkynyl are unsubstituted or bear one or more identical or different Z^{3a} substituents, C₃-C₁₂-cycloalkyl is unsubstituted or bears one or more identical or different Z^{3b} substituents, and C₆-C₁₄-aryl is unsubstituted or bears one or more identical or different Z^{3Ar} substituents;
R³¹, R³², R³³, R³⁴, R³⁵, R³⁶, R³⁷ and R³⁸ are each independently selected from hydrogen, cyano, bromine and chlorine,
   with the proviso that 1, 2, 3, 4, 5, 6, 7 or 8 of the R³¹, R³², R³³, R³⁴, R³⁵, R³⁶, R³⁷ or R³⁸ substituents are cyano;
   where
   R³⁹ is hydrogen, C₁-C₁₀-alkyl, C₂-C₁₀-alkenyl, C₂-C₁₀-alkynyl, C₃-C₁₂-cycloalkyl or C₆-C₁₄-aryl, where
      C₁-C₁₀-alkyl, C₂-C₁₀-alkenyl, C₂-C₁₀-alkynyl are unsubstituted or bear one or more identical or different R^{3a} substituents,
      C₃-C₁₂-cycloalkyl is unsubstituted or bears one or more identical or different R^{3b} substituents and
      C₆-C₁₄-aryl is unsubstituted or bears one or more identical or different R^{3Ar} substituents;
   R³¹⁰ and R³¹¹ are each independently hydrogen, C₁-C₁₀-alkyl, C₂-C₁₀-alkenyl, C₂-C₁₀-alkynyl, C₃-C₁₂-cycloalkyl or C₆-C₁₄-aryl, where C₁-C₁₀-alkyl, C₂-C₁₀-alkenyl, C₂-C₁₀-alkynyl are unsubstituted or bear one or more identical or different R^{3a} substituents, C₃-C₁₂-cycloalkyl is unsubstituted or bears one or more identical or different R^{3b} substituents and C₆-C₁₄-aryl is unsubstituted or bears one or more identical or different R^{3Ar} substituents;
   each Z^{3a} is independently halogen, hydroxyl, NR^{310a}R^{311a}, C₁-C₁₀-alkoxy, C₁-C₁₀-haloalkoxy, C₁-C₁₀-alkylthio, C₃-C₁₂-cycloalkyl, C₆-C₁₄-aryl, C(═O)R^{39a}; C(═O)OR^{39a} or C(O)NR^{310a}R^{311a}, where C₃-C₁₂-cycloalkyl is unsubstituted or bears one or more identical or different R^{3b} substituents and C₆-C₁₄-aryl is unsubstituted or bears one or more identical or different R^{3Ar} substituents;
   each Z^{3b} and each Z^{3Ar} is independently halogen, hydroxyl, NR^{310a}R^{311a}, C₁-C₁₀-alkyl, C₁-C₁₀-alkoxy, C₁-C₁₀-haloalkoxy, C₁-C₁₀-alkylthio, C(═O)R^{39a}; C(═O)OR^{39a} or C(O)NR^{310a}R^{311a};
   each R^{3a} is independently halogen, hydroxyl, C₁-C₁₀-alkoxy, C₃-C₁₂-cycloalkyl or C₆-C₁₄-aryl;
   each R^{3b} is independently halogen, hydroxyl, C₁-C₁₀-alkyl, C₁-C₁₀-alkoxy, C₁-C₁₀-haloalkoxy, C₁-C₁₀-alkylthio, C₂-C₁₀-alkenyl, C₂-C₁₀-alkynyl, C₃-C₁₂-cycloalkyl or C₆-C₁₄-aryl;
   each R^{3Ar} is independently halogen, hydroxyl, C₁-C₁₀-alkyl, C₁-C₁₀-alkoxy, C₁-C₁₀-haloalkoxy, C₁-C₁₀-alkylthio, C₂-C₁₀-alkenyl, C₂-C₁₀-alkynyl, C₃-C₁₂-cycloalkyl or C₆-C₁₄-aryl;
   R^{39a} is hydrogen, C₁-C₁₀-alkyl, C₂-C₁₀-alkenyl, C₂-C₁₀-alkynyl, C₃-C₁₂-cycloalkyl or C₆-C₁₄-aryl; and
   R^{310a}, R^{311a} are each independently hydrogen, C₁-C₁₀-alkyl, C₂-C₁₀-alkenyl, C₂-C₁₀-alkynyl, C₃-C₁₂-cycloalkyl or C₆-C₁₄-aryl,
   and mixtures thereof;

### (B3) a cyanated compound of formula (IV)

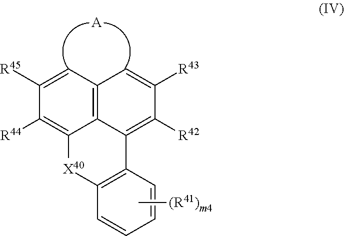

wherein
m4 is 0, 1, 2, 3 or 4;
each R⁴¹ independently from each other is selected from bromine, chlorine, cyano, —NR^{4a}R^{4b}, C₁-C₂₄-alkyl, C₁-C₂₄-haloalkyl, C₁-C₂₄-alkoxy, C₁-C₂₄-haloalkoxy, C₃-C₂₄-cycloalkyl, heterocycloalkyl, heteroaryl, C₆-C₂₄-aryl, C₆-C₂₄-aryloxy, C₆-C₂₄-aryl-C₁-C₁₀-alkylene, where the rings of cycloalkyl, heterocycloalkyl, heteroaryl, aryl, aryloxy in the six last-mentioned radicals are unsubstituted or substituted with 1, 2, 3, 4 or 5 identical or different radicals R^{41a} and where C₁-C₂₄-alkyl, C₁-C₂₄-haloalkyl, C₁-C₂₄-alkoxy, and the a1-kylene moiety of C₆-C₂₄-aryl-C₁-C₁₀-alkylene may be interrupted by one or more groups selected from O, S and NR^{4c};
   at least one of the radicals R⁴², R⁴³, R⁴⁴ and R⁴⁵ is CN, and the remaining radicals, independently from each other, are selected from hydrogen, chlorine and bromine;
X⁴⁰ is O, S, SO or SO₂;
A is a diradical selected from diradicals of the general formulae (A.1), (A.2), (A.3), and (A.4)

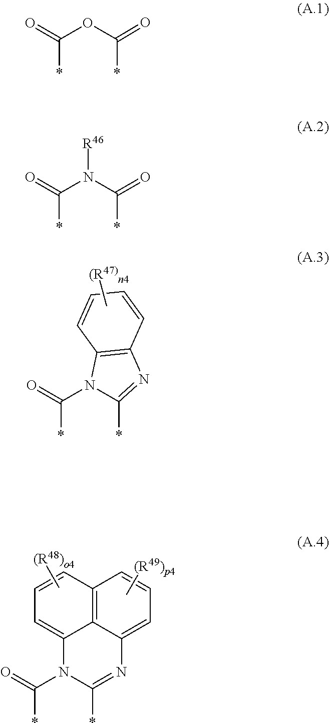

wherein
in each case denotes the point of attachments to the remainder of the molecule;
n4 is 0, 1, 2, 3 or 4;
o4 is 0, 1, 2 or 3;
p4 is 0, 1, 2 or 3;
R⁴⁶ is hydrogen, C₁-C₂₄-alkyl, C₁-C₂₄-haloalkyl, C₃-C₂₄-cycloalkyl, C₆-C₂₄-aryl or C₆-C₂₄-aryl-C₁-C₁₀-alkylene, where the rings of cycloalkyl, aryl, and aryl-alkylene in the three last-mentioned radicals are unsubstituted or substituted with 1, 2, 3, 4 or 5 identical or different radicals R^{46a}, and where C₁-C₂₄-alkyl, C₁-C₂₄-haloalkyl and the alkylene moiety of C₆-C₂₄-aryl-C₁-C₁₀-alkylene may be interrupted by one or more heteroatoms or heteroatomic groups selected from O, S and NR^{4c};
each R⁴⁷ independently from each other is selected from bromine, chlorine, cyano, —NR^{4a}R^{4b}, C₁-C₂₄-alkyl, C₁-C₂₄-haloalkyl, C₁-C₂₄-alkoxy, C₁-C₂₄-haloalkoxy, C₃-C₂₄-cycloalkyl, heterocycloalkyl, heteroaryl, C₆-C₂₄-aryl, C₆-C₂₄-aryloxy, C₆-C₂₄-aryl-C₁-C₁₀-alkylene, where the rings of cycloalkyl, heterocycloalkyl, heteroaryl, aryl and aryl-alkylene in the six last-mentioned radicals are unsubstituted or substituted with 1, 2, 3, 4 or 5 identical or different radicals R^{47a} and where C₁-C₂₄-alkyl, C₁-C₂₄-haloalkyl, C₁-C₂₄-alkoxy, C₁-C₂₄-haloalkoxy, and the alkylene moiety of C₆-C₂₄-aryl-C₁-C₁₀-alkylene may be interrupted by one or more groups selected from O, S and NR^{4c};
each R⁴⁸ independently from each other is selected from bromine, chlorine, cyano, NR^{4a}R^{4b}, C₁-C₂₄-alkyl, C₁-C₂₄-haloalkyl, C₁-C₂₄-alkoxy, C₁-C₂₄-haloalkoxy, C₃-C₂₄-cycloalkyl, heterocycloalkyl, heteroaryl, C₆-C₂₄-aryl, C₆-C₂₄-aryloxy, C₆-C₂₄-aryl-C₁-C₁₀-alkylene, where the rings of cycloalkyl, heterocycloalkyl, heteroaryl, aryl and aryl-alkylene in the six last-mentioned radicals are unsubstituted or substituted with 1, 2, 3, 4 or 5 identical or different radicals R^{48a} and where C₁-C₂₄-alkyl, C₁-C₂₄-haloalkyl, C₁-C₂₄-alkoxy, C₁-C₂₄-haloalkoxy, and the alkylene moiety of C₆-C₂₄-aryl-C₁-C₁₀-alkylene may be interrupted by one or more groups selected from O, S and NR^{4c};
each R⁴⁹ independently from each other is selected from bromine, chlorine, cyano, NR^{4a}R^{4b}, C₁-C₂₄-alkyl, C₁-C₂₄-haloalkyl, C₁-C₂₄-alkoxy, C₁-C₂₄-haloalkoxy, C₃-C₂₄-cycloalkyl, heterocycloalkyl, heteroaryl, C₆-C₂₄-aryl, C₆-C₂₄-aryloxy, C₆-C₂₄-aryl-C₁-C₁₀-alkylene, where the rings of cycloalkyl, heterocycloalkyl, heteroaryl, aryl and aryl-alkylene in the six last-mentioned radicals are unsubstituted or substituted with 1, 2, 3, 4 or 5 identical or different radicals R^{49a} and where C₁-C₂₄-alkyl, C₁-C₂₄-haloalkyl, C₁-C₂₄-alkoxy, C₁-C₂₄-haloalkoxy, and the alkylene moiety of C₆-C₂₄-aryl-C₁-Cₙ-alkylene may be interrupted by one or more groups selected from O, S and NR^{4c};
R⁴1a, R^{46a}, R^{47a}, R^{48a}, R^{49a} are independently of one another selected from C₁-C₂₄-alkyl, C₁-C₂₄-fluoroalkyl, C₁-C₂₄-alkoxy, fluorine, chlorine and bromine;
R^{4a}, R^{4b}, R^{4c} are independently of one another are selected from hydrogen, C₁-C₂₀-alkyl, C₃-C₂₄-cycloalkyl, heterocycloalkyl, heteroaryl and C₆-C₂₄-aryl;
and mixtures thereof;

### (B4) a benz(othi)oxanthene compound of formula (V)

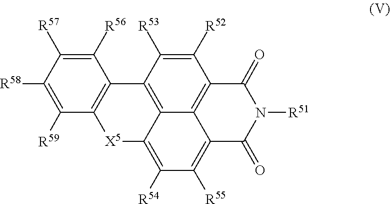

wherein
X⁵ is oxygen or sulfur;
R⁵¹ is phenyl which is unsubstituted or carries 1, 2, 3, 4, or 5 substituents selected from halogen, R⁵¹¹, OR⁵⁵², NHR⁵⁵² and NR⁵⁵²R⁵⁵⁷;
R⁵², R⁵³, R⁵⁴, R⁵⁵, R⁵⁶, R⁵⁷, R⁵⁸ and R⁵⁹ are independently of each other selected from hydrogen, halogen, R⁵⁵³, OR⁵⁵³, NH R⁵⁵³ and NR⁵⁵³R⁵⁵4,
wherein
R⁵¹¹ is selected from C₁-C₂₄-alkyl, C₆-C₂₄-aryl and heteroaryl;
R⁵⁵² and R⁵⁵⁷ are independently of each other selected from C₁-C₁₈-alkyl, C₆-C₂₄-aryl and heteroaryl; and
R⁵⁵³ and R⁵⁵⁴ are independently of each other selected from C₁-C₁₈-alkyl, C₆-C₂₄-aryl and heteroaryl;
and mixtures thereof;

### (B5) A Benzimidazoxanthenisoquinoline Compound of Formulae (VIA) or (VIB)

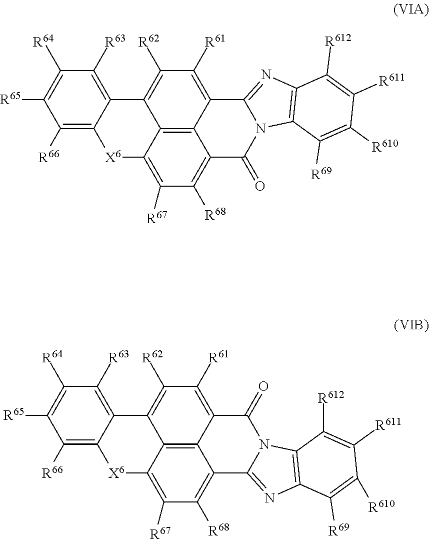

wherein
X⁶ is oxygen or sulfur;
R⁶¹, R⁶², R⁶³, R⁶⁴, R⁶⁵, R⁶⁶, R⁶⁷, R⁶⁸, R⁶⁹, R⁶¹⁰, R⁶¹¹ and R⁶¹² are independently of each other selected from hydrogen, halogen, R⁶⁶¹, OR⁶⁶¹, NHR⁶⁶¹ and NR⁶⁶¹R⁶⁶²,
wherein
each R⁶⁶¹ is selected from C₁-C₁₃-alkyl, C₆-C₂₄-aryl and heteroaryl; and
each R⁶⁶² is selected from C₁-C₁₃-alkyl, C₆-C₂₄-aryl and heteroaryl;
and mixtures thereof;

### (B6) Fluorescent Compound Comprising at Least One Structural Unit of Formula (VII)

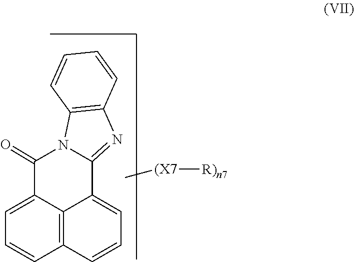

where one or more CH groups of the six-membered ring of the benzimidazole structure shown may be replaced by nitrogen and where the symbols are each defined as follows:
n7 is a number from 0 to (10-p7) for each structural unit of formula (VII); where p7 is the number of CH units which have been replaced by nitrogen in the six-membered ring of the benzimidazole structure shown,
X7 is a chemical bond, O, S, SO, SO₂, NR⁷¹; and
R is an aliphatic radical, cycloaliphatic radical, aryl, heteroaryl, each of which may bear substituents,
   an aromatic or heteroaromatic ring or ring system, each of which is fused to other aromatic rings of the structural unit of formula (VII), is F, Cl, Br, CN, H when X7 is not a chemical bond;
   where two R radicals may be joined to give one cyclic radical and
   where X7 and R, when n7>one, may be the same or different;
   R⁷¹ is each independently hydrogen, C₁-C₁₃-alkyl or cycloalkyl, the carbon chain of which may comprise one or more —O—, —S—, —CO—, —SO— and/or —SO₂— moieties and which may be mono- or polysubstituted;
      aryl or heteroaryl which may be mono- or polysubstituted;
   and mixtures thereof;

### ((B7) a Perylene Compound of Formulae (VIII) or (IX)

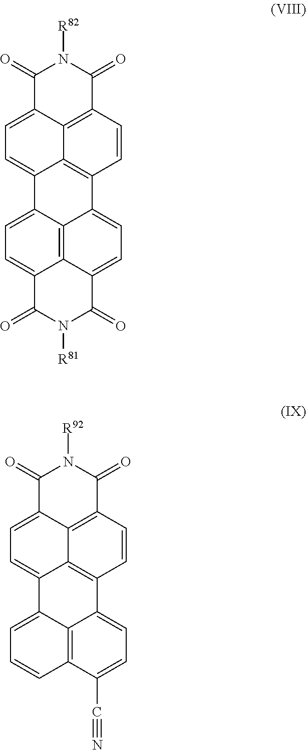

where
R⁸¹, R⁸² are each independently C₁-C₃₀-alkyl, C₂-C₃₀-alkyl which is interrupted by one or more oxygen, C₃-C₈-cycloalkyl, C₆-C₁₀-aryl, heteroaryl, C₆-C₁₀-aryl-C₁-C₁₀-alkylene, where the aromatic ring in the three latter radicals is unsubstituted or mono- or polysubstituted by C₁-C₁₀-alkyl;
R⁹² is C₁-C₃₀-alkyl, C₃-C₈-cycloalkyl, aryl, heteroaryl, aryl-C₁-C₁₀-alkylene, where the aromatic ring in the three latter radicals is unsubstituted or mono- or polysubstituted by C₁-C₁₀-alkyl;

### (B8) A Naphthalene Monoimide Compound of Formula (X)

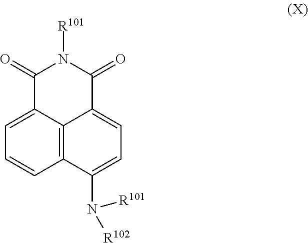

wherein
each R¹⁰¹ independently of each other is hydrogen, C₁-C₃₀-alkyl, C₂-C₃₀-alkyl which is interrupted by one or more oxygen, C₃-C₈-cycloalkyl, C₆-C₁₀-aryl, heteroaryl, C₆-C₁₀-aryl-C₁-C₁₀-alkylene, where the aromatic ring in the three latter radicals is unsubstituted or mono- or polysubstituted by C₁-C₁₀-alkyl;
R¹⁰² is hydrogen, C₁-C₃₀-alkyl, C₂-C₃₀-alkyl which is interrupted by one or more oxygen, C₃-C₅-cycloalkyl, C₆-C₁₀-aryl, heteroaryl, C₆-C₁₀-aryl-C₁-C₁₀-alkylene, where the aromatic ring in the three latter radicals is unsubstituted or mono- or polysubstituted by C₁-C₁₀-alkyl

### (B9) 7-(diethylamino)-3-(5-methylbenzo[d]oxazol-2-yl)-2H-chromen-2-one

### (B10) a perylene compound of formulae (XIA) or (XIB)

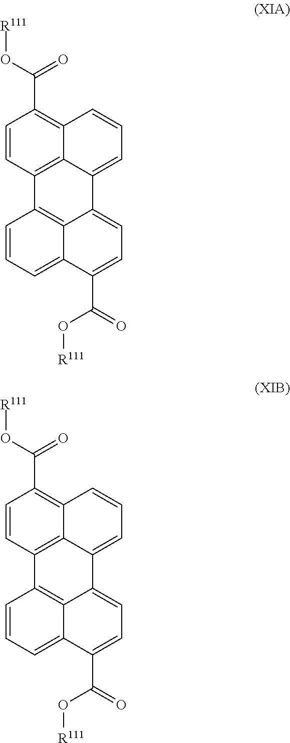

wherein
each R¹¹¹ independently of each other is C₁-C₁₈ alkyl, C₄-C₈ cycloalkyl, which may be mono- or polysubstituted by halogen or by linear or branched C₁-C₁₈ alkyl, or phenyl or naphthyl which may be mono- or polysubstituted by halogen or by linear or branched C₁-C₁₈ alkyl;
and mixtures thereof;

### (B11) a cyanated perylene compound of formulae (XIIA) or (XIIB)

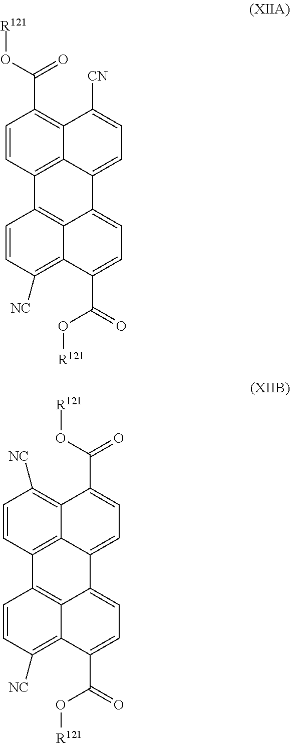

wherein
each R¹²¹ independently of each other is C₁-C₁₈ alkyl, C₄-C₈ cycloalkyl, which may be mono- or polysubstituted by halogen or by linear or branched C₁-C₁₈ alkyl, or phenyl or naphthyl which may be mono- or polysubstituted by halogen or by linear or branched C₁-C₁₈ alkyl;
and mixtures thereof;

### (B12) a naphthoylbenzimidazole compound of formula (XIII)

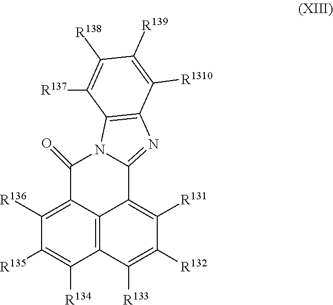

wherein
at least one of the radicals R¹³¹, R¹³², R¹³³, R¹³⁴, R¹³⁵, R¹³⁶, R¹³⁷, R¹³⁸, R¹³⁹ and R¹³¹⁰ independently of each other is aryl which carries one, two or three cyano groups and 0, 1, 2, 3 or 4 substituents R^{Ar13} and the remaining radicals R¹³¹, R¹³², R¹³³, R¹³⁴, R¹³⁵, R¹³⁶, R¹³⁷, R¹³⁸, R¹³⁹ and R¹³¹⁰ independently of each other are selected from hydrogen and aryl which is unsubstituted or carries 1, 2, 3, 4 or 5 substituents R^{Ar13}, where
R^{Ar13} independently of each other and independently of each occurrence is selected from halogen,
   C₁-C₃₀-alkyl, C₂-C₃₀-alkenyl, C₂-C₃₀-alkynyl, where the three latter radicals are unsubstituted or carry one or more R^{13a} groups,
   C₃-C₅-cycloalkyl, 3- to 8-membered heterocyclyl, where the two latter radicals are unsubstituted or carry one or more R^{13b} groups, aryl and heteroaryl, where the two latter radicals are unsubstituted or carry one or more R^{13c} groups,
   where
   R^{13a} independently of each other and independently of each occurrence is selected from cyano, halogen, C₃-C₅-cycloalkyl, 3- to 8-membered heterocyclyl, aryl and heteroaryl, where C₃-C₅-cycloalkyl, 3- to 8-membered heterocyclyl are unsubstituted or bear one or more R^{13b}1 groups, and where aryl and heteroaryl are unsubstituted or bear one or more R^{13c1} groups;
   R^{13b} independently of each other and independently of each occurrence is selected from cyano, halogen, C₁-C₁₃-alkyl, C₃-C₅-cycloalkyl, 3- to 8-membered heterocyclyl, aryl and heteroaryl, where C₃-C₅-cycloalkyl, 3- to 8-membered heterocyclyl are unsubstituted or bear one or more R^{13b}1 groups, and where aryl and heteroaryl are unsubstituted or bear one or more R^{13c1} groups;
   R^{13c} independently of each other and independently of each occurrence is selected from cyano, halogen, C₁-C₁₃-alkyl, C₃-C₅-cycloalkyl, 3- to 8-membered heterocyclyl, aryl and heteroaryl, where C₃-C₅-cycloalkyl, 3- to 8-membered heterocyclyl are unsubstituted or bear one or more R^{13b}1 groups, and where aryl and heteroaryl are unsubstituted or bear one or more R^{13c1} groups; where
      R^{13b}1 independently of each other and independently of each occurrence is selected from halogen, C₁-C₁₃-alkyl and C₁-C₁₈-haloalkyl,
      R^{13c1} independently of each other and independently of each occurrence is selected from halogen, C₁-C₁₃-alkyl and C₁-C₁₈-haloalkyl;
and mixtures thereof;

### (B13) a Perylene Compound of Formula (XIV)

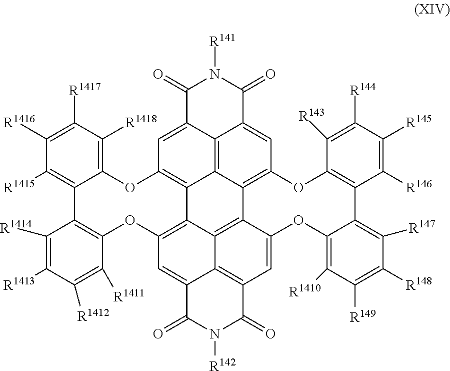

wherein
R¹⁴¹ and R¹⁴², independently of each other, are selected from hydrogen, in each case unsubstituted or substituted C₁-C₃₀-alkyl, polyalkyleneoxy, C₁-C₃₀-alkoxy, C₁-C₃₀-alkylthio, C₃-C₂₀-cycloalkyl, C₃-C₂₀-cycloalkyloxy, C₆-C₂₄-aryl and C₆-C₂₄-aryloxy;
R¹⁴³, R¹⁴⁴, R¹⁴⁵, R¹⁴⁶, R¹⁴⁷, R¹⁴⁸, R¹⁴⁹, R¹⁴¹⁰, R¹⁴¹¹, R¹⁴¹², R¹⁴¹³, R¹⁴¹⁴, R¹⁴¹⁵, R¹⁴¹⁶, R¹⁴¹⁷ and R¹⁴¹⁸ independently of each other, are selected from hydrogen, halogen, cyano, hydroxyl, mercapto, nitro, —NE¹⁴¹E¹⁴², —NR^{Ar141}COR^{Ar142}, —CONR^{Ar141}R^{Ar142}, —S₂NR^{Ar141}R^{Ar142}, —COR^{Ar141}, —S₃R^{Ar142}, in each case unsubstituted or substituted C₁-C₃₀-alkyl, polyalkyleneoxy, C₁-C₃₀-alkoxy, C₁-C₃₀-alkylthio, C₃-C₂₀-cycloalkyl, C₃-C₂₀-cycloalkoxy, C₆-C₂₄-aryl, C₆-C₂₄-aryloxy and C₆-C₂₄-arylthio,
where R¹⁴³ and R¹⁴⁴, R¹⁴⁴ and R¹⁴⁵, R¹⁴⁵ and R¹⁴⁶, R¹⁴⁶ and R¹⁴⁷, R¹⁴⁷ and R¹⁴⁸, R¹⁴⁸ and R¹⁴⁹, R¹⁴⁹ and R¹⁴¹⁰, R¹⁴¹¹ and R¹⁴¹², R¹⁴¹² and R¹⁴¹³, R¹⁴¹³ and R¹⁴¹⁴, R¹⁴¹⁴ and R¹⁴¹⁵, R¹⁴¹⁵ and R¹⁴¹⁶, R¹⁴¹⁶ and R¹⁴¹⁷ and/or R¹⁴¹⁷ and R¹⁴¹⁸ together with the carbon atoms of the biphenylyl moiety to which they are bonded, may also form a further fused aromatic or non-aromatic ring system wherein the fused ring system is unsubstituted or substituted;
   where
   E¹⁴¹ and E¹⁴², independently of each other, are hydrogen, unsubstituted or substituted C₁-C₁₈-alkyl, unsubstituted or substituted C₂-C₁₈-alkenyl, unsubstituted or substituted C₂-C₁₈-alkynyl, unsubstituted or substi-tuted C₃-C₂₀-cycloalkyl or unsubstituted or substituted C₆-C₁₀-aryl;
      R^{Ar141} and R^{Ar142}, each independently of each other, are hydrogen, unsubstituted or substituted C₁-C₁₈-alkyl, unsubstituted or substituted C₃-C₂₀-cycloalkyl, unsubstituted or substituted heterocyclyl, unsubstituted or substituted C₆-C₂₀-aryl or unsubstituted or substituted heteroaryl;
and mixtures thereof;

### (B14) a perylene bisimide compound of formula (XV)

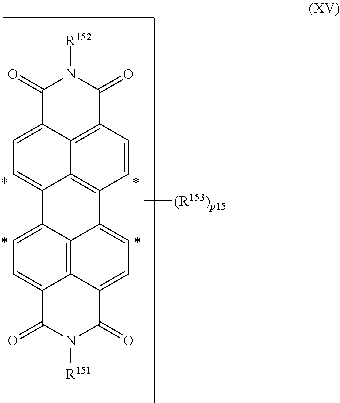

wherein
p15 is 1, 2, 3 or 4;
R¹⁵¹ and R¹⁵² independently of each other are C₁-C₁₀-alkyl, which is unsubstituted or substituted by C₆-C₁₀-aryl which in turn is unsubstituted or substituted by 1, 2 or 3 C₁-C₁₀-alkyl,
   C₂-C₂₀-alkyl, which is interrupted by one or more oxygen,
   C₃-C₅-cycloalkyl, which is unsubstituted or substituted by 1, 2 or 3 C₁-C₁₀-alkyl, or
   C₆-C₁₀-aryl which is unsubstituted or substituted by 1, 2 or 3 C₁-C₁₀-alkyl;
each R¹⁵³ independently of each other is fluorine, chlorine, C₁-C₁₆-alkyl, C₂-C₁₆-alkyl interrupted by one or more oxygen, C₁-C₁₆-alkoxy, C₆-C₁₀-aryloxy which is unsubstituted or mono- or polysubstituted by fluorine, chlorine, C₁-C₁₆-alkyl, C₂-C₁₆-alkyl interrupted by one or more oxygen, C₁-C₁₆-alkoxy or C₆-C₁₀-aryl, which is unsubstituted or substituted by 1, 2 or 3 radicals selected from C₁-C₆-alkyl, C₁-C₆-alkoxy-C₁-C₆-alkyl and C₁-C₆-alkoxy, where the R¹³³ radicals are at the positions indicated by *;
and mixtures thereof;

### (B15) A Cyanoaryl Substituted Compound of Formula (XVI)

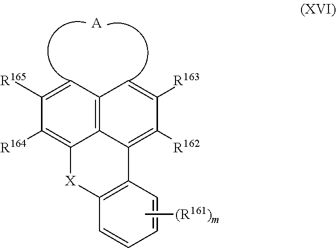

wherein
m is 0, 1, 2, 3 or 4;
each R¹⁶¹ independently from each other is selected from bromine, chlorine, cyano, —NR^{16a}R^{16b}, C₁-C₂₄-alkyl, C₁-C₂₄-haloalkyl, C₁-C₂₄-alkoxy, C₁-C₂₄-haloalkoxy, C₃-C₂₄-cycloalkyl, heterocycloalkyl, heteroaryl, C₆-C₂₄-aryl, C₆-C₂₄-aryloxy, C₆-C₂₄-aryl-C₁-C₁₀-alkylene, where the rings of cycloalkyl, heterocycloalkyl, heteroaryl, aryl, aryloxy and -aryl-alkylene in the six last-mentioned radicals are unsubstituted or substituted with 1, 2, 3, 4 or 5 identical or different radicals R^{1a} and where C₁-C₂₄-alkyl, C₁-C₂₄-haloalkyl, C₁-C₂₄-alkoxy, and the alkylene moiety of C₆-C₂₄-aryl-C₁-C₁₀-alkylene may be interrupted by one or more groups selected from 0, S and NR^{16c};
R¹⁶², R¹⁶³, R¹⁶⁴ and R¹⁶⁵ are selected from hydrogen, chlorine, bromine, and C₆-C₂₄-aryl, which carries one, two or three cyano groups;
with the proviso that at least one of the radicals R¹⁶¹, R¹⁶², R¹⁶³, R¹⁶⁴ and R¹⁶⁵ is C₆-C₂₄-aryl, which carries one, two or three cyano groups;
X is O, S, SO or SO₂;
A is a diradical selected from diradicals of the general formulae (A. 161), (A.162), (A.163), and (A.164)

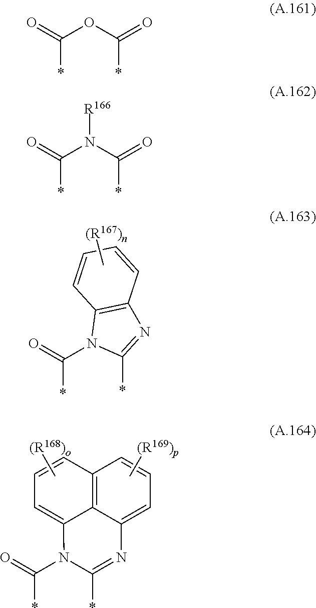

wherein
* in each case denotes the point of attachments to the remainder of the molecule;
n is 0, 1, 2, 3 or 4;
o is 0, 1, 2 or 3;
p is 0, 1, 2 or 3;
R¹⁶⁶ is hydrogen, C₁-C₂₄-alkyl, C₁-C₂₄-haloalkyl, C₃-C₂₄-cycloalkyl, C₆-C₂₄-aryl or C₆-C₂₄-aryl-C₁-C₁₀-alkylene, where the rings of cycloalkyl, aryl, and aryl-alkylene in the three last-mentioned radicals are unsubstituted or substituted with 1, 2, 3, 4 or 5 identical or different radicals R^{166a}, and where C₁-C₂₄-alkyl, C₁-C₂₄-haloalkyl and the alkylene moiety of C₆-C₂₄-aryl-C₁-C₁₀-alkylene may be interrupted by one or more heteroatoms or heteroatomic groups selected from O, S and NR^{16c};
each R¹⁶⁷ independently from each other is selected from bromine, chlorine, cyano, —NR^{16a}R^{16b}, C₁-C₂₄-alkyl, C₁-C₂₄-haloalkyl, C₁-C₂₄-alkoxy, C₁-C₂₄-haloalkoxy, C₃-C₂₄-cycloalkyl, heterocycloalkyl, heteroaryl, C₆-C₂₄-aryl, C₆-C₂₄-aryloxy, C₆-C₂₄-aryl-C₁-C₁₀-alkylene, where the rings of cycloalkyl, heterocycloalkyl, heteroaryl, aryl and aryl-alkylene in the six last-mentioned radicals are unsubstituted or substituted with 1, 2, 3, 4 or 5 identical or different radicals R^{167a} and where C₁-C₂₄-alkyl, C₁-C₂₄-haloalkyl, C₁-C₂₄-alkoxy, C₁-C₂₄-haloalkoxy, and the alkylene moiety of C₆-C₂₄-aryl-C₁-C₁₀-alkylene may be interrupted by one or more groups selected from O, S and NR^{16c};
each R¹⁶⁸ independently from each other is selected from bromine, chlorine, cyano, NR^{16a}R^{16b}, C₁-C₂₄-alkyl, C₁-C₂₄-haloalkyl, C₁-C₂₄-alkoxy, C₁-C₂₄-haloalkoxy, C₃-C₂₄-cycloalkyl, heterocycloalkyl, heteroaryl, C₆-C₂₄-aryl, C₆-C₂₄-aryloxy, C₆-C₂₄-aryl-C₁-C₁₀-alkylene, where the rings of cycloalkyl, heterocycloalkyl, heteroaryl, aryl and aryl-alkylene in the six last-mentioned radicals are unsubstituted or substituted with 1, 2, 3, 4 or 5 identical or different radicals R^{168a} and where C₁-C₂₄-alkyl, C₁-C₂₄-haloalkyl, C₁-C₂₄-alkoxy, C₁-C₂₄-haloalkoxy, and the alkylene moiety of C₆-C₂₄-aryl-C₁-C₁₀-alkylene may be interrupted by one or more groups selected from O, S and NR^{16c};
each R¹⁶⁹ independently from each other is selected from bromine, chlorine, cyano, NR^{16a}R^{16b}, C₁-C₂₄-alkyl, C₁-C₂₄-haloalkyl, C₁-C₂₄-alkoxy, C₁-C₂₄-haloalkoxy, C₃-C₂₄-cycloalkyl, heterocycloalkyl, heteroaryl, C₆-C₂₄-aryl, C₆-C₂₄-aryloxy, C₆-C₂₄-aryl-C₁-C₁₀-alkylene, where the rings of cycloalkyl, heterocycloalkyl, heteroaryl, aryl and aryl-alkylene in the six last-mentioned radicals are unsubstituted or substituted with 1, 2, 3, 4 or 5 identical or different radicals R^{169a} and where C₁-C₂₄-alkyl, C₁-C₂₄-haloalkyl, C₁-C₂₄-alkoxy, C₁-C₂₄-haloalkoxy and the alkylene moiety of C₆-C₂₄-aryl-C₁-C₁₀-alkylene may be interrupted by one or more groups selected from O, S and NR^{16c};
   where
   R^{161a}, R^{166a}, R^{167a}, R^{168a}, R^{169a} are independently of one another selected from C₁-C₂₄-alkyl, C₁-C₂₄-fluoroalkyl, C₁-C₂₄-alkoxy, fluorine, chlorine, bromine and cyano;
   R^{16a}, R^{16b}, R^{16c} are independently of one another are selected from hydrogen, C₁-C₂₀-alkyl, C₃-C₂₄-cycloalkyl, heterocycloalkyl, hetaryl and C₆-C₂₄-aryl;
and mixtures thereof.

Preferably, the colorants from groups (B1) to (B15) are organic fluorescent dyes. The colorants of groups (B1) to (B15) can absorb light comprising a wavelength in the range from 400 to less than 680 nm and emit the absorbed light at a wavelength of greater than 500 to less than 680 nm. For example, said wavelength range is relevant to plant photophysiology.

### Colorant (B1)

Cyanated naphthoylbenzimidazole compound of formula (II) are known from WO 2015/019270. Compounds of formula (II) are usually green fluorescent dyes. With regard to the use in the converter of the present invention, the compound (II) is preferably selected from a compound of formula (II-A)

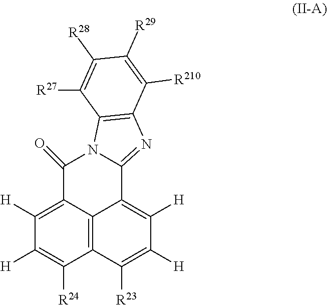

and mixtures thereof,
in which
R²³ and R²⁴ are each independently cyano, phenyl, 4-cyanophenyl or phenyl which carries 1, 2 or 3 substituents selected from C₁-C₁₀-alkyl, especially cyano, phenyl or 4-cyanophenyl; and
R²⁷, R²⁸, R²⁹ and R²¹⁰ are each independently hydrogen, cyano, phenyl, 4-cyanophenyl or phenyl which carries 1, 2 or 3 substituents selected from C₁-C₁₀-alkyl, especially hydrogen, cyano, phenyl or 4-cyanophenyl.

More preferred are the compounds specified in WO 2015/019270 on page 16, 2^{nd} paragraph to page 20, 3^{rd} paragraph. With regard to the use in the converter of the present invention, especially preferred are compounds of formula (II) selected from com-pounds of formulae (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II-35), (II-36), (II-37), (II-38), (II-39), (II-40), (II-41), (II-42), (II-43), (II-44), (II-45), (II-46), (II-47), (II-48), (II-49), (II-50) or mixtures thereof

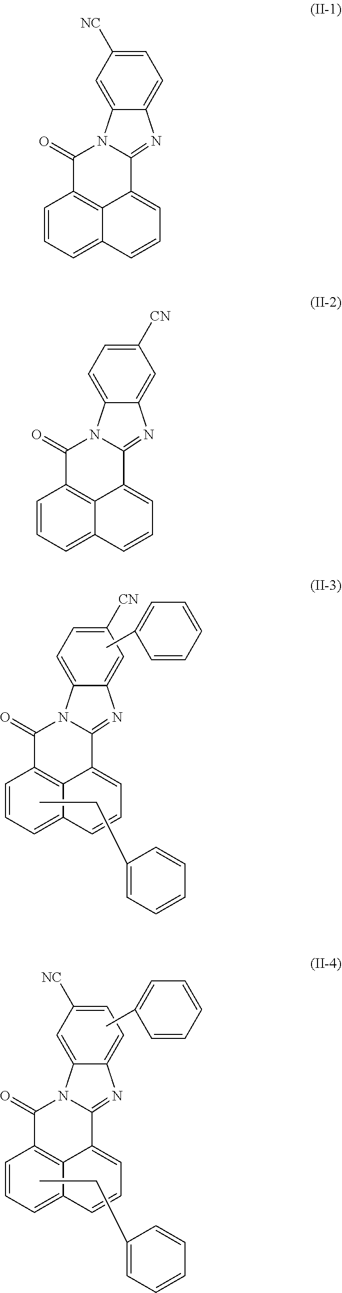

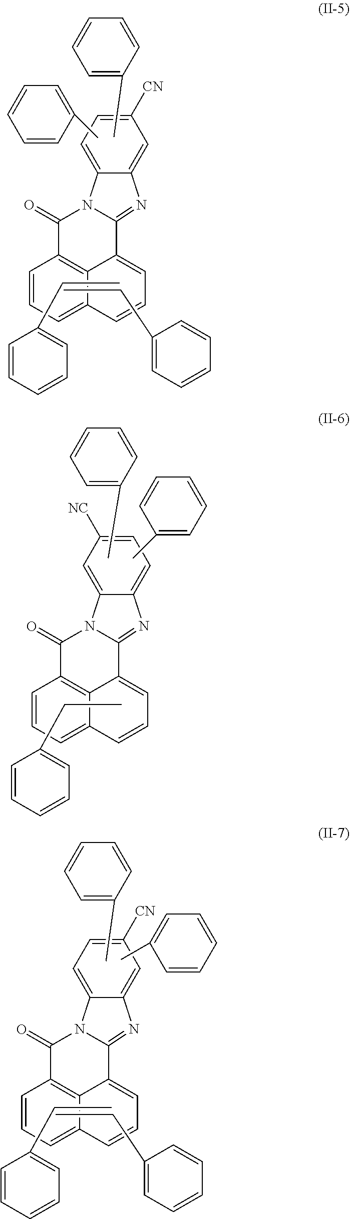

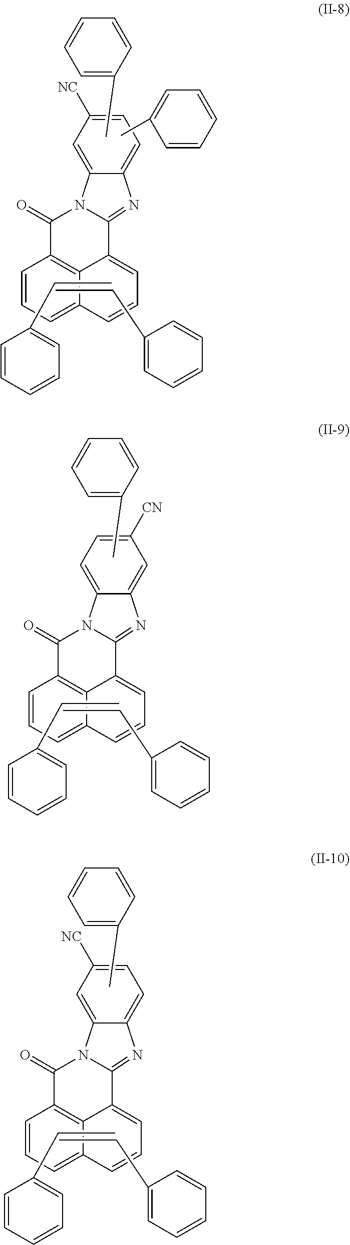

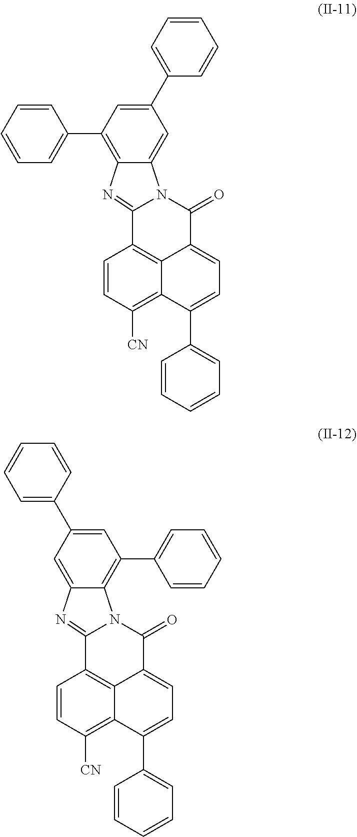 [SMILES: N#Cc1ccc2c3c1c(-c1ccccc1)ccc3c(=O)n1c3cc(-c4ccccc4)cc(-c4ccccc4)c3nc21 ; N#Cc1ccc2c3c1c(-c1ccccc1)ccc3c(=O)n1c2nc2cc(-c3ccccc3)cc(-c3ccccc3)c21]

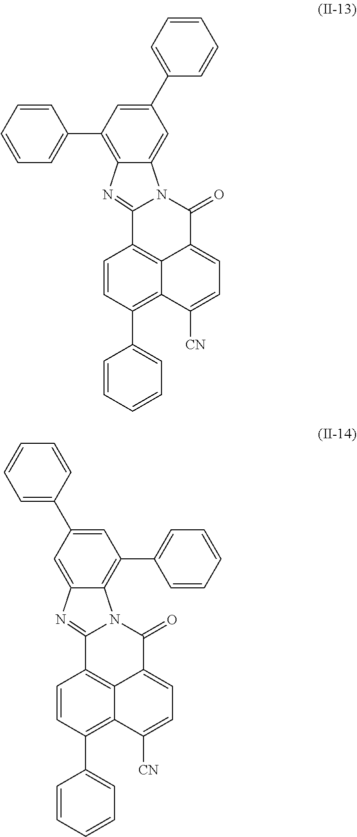 [SMILES: N#Cc1ccc2c(=O)n3c4cc(-c5ccccc5)cc(-c5ccccc5)c4nc3c3ccc(-c4ccccc4)c1c23 ; N#Cc1ccc2c(=O)n3c(nc4cc(-c5ccccc5)cc(-c5ccccc5)c43)c3ccc(-c4ccccc4)c1c23]

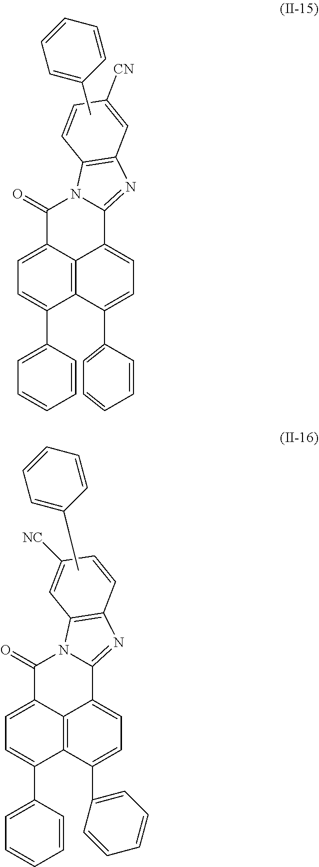

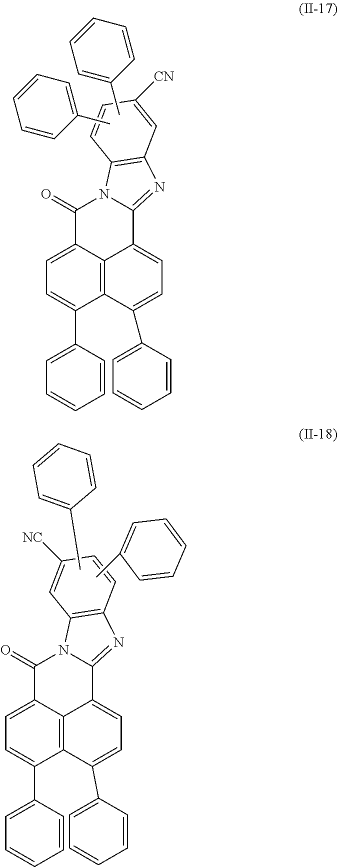

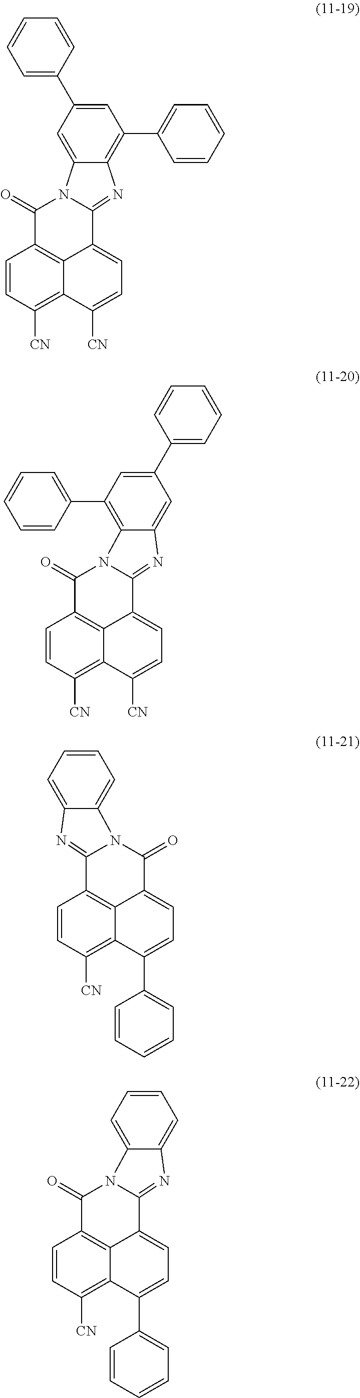

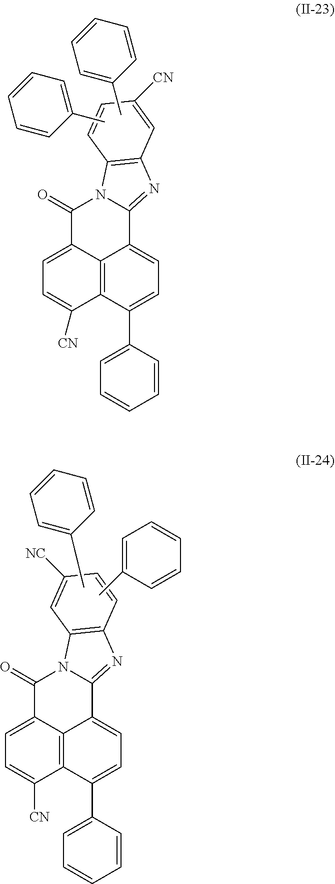

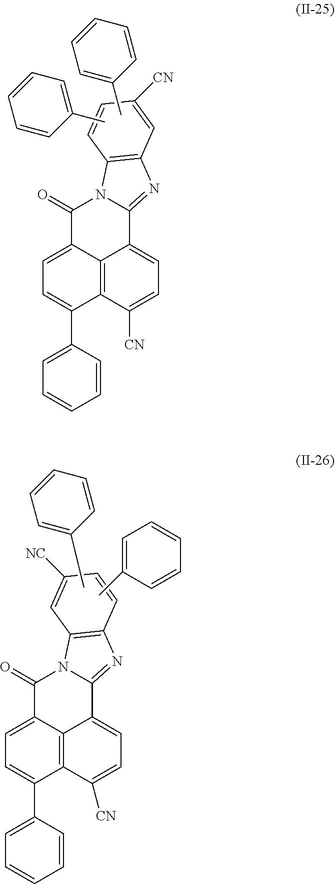

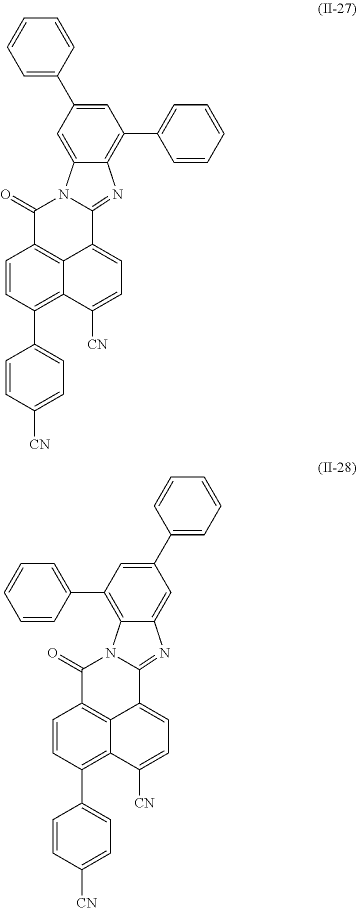 [SMILES: N#Cc1ccc(-c2ccc3c(=O)n4c5cc(-c6ccccc6)cc(-c6ccccc6)c5nc4c4ccc(C#N)c2c34)cc1 ; N#Cc1ccc(-c2ccc3c(=O)n4c(nc5cc(-c6ccccc6)cc(-c6ccccc6)c54)c4ccc(C#N)c2c34)cc1]

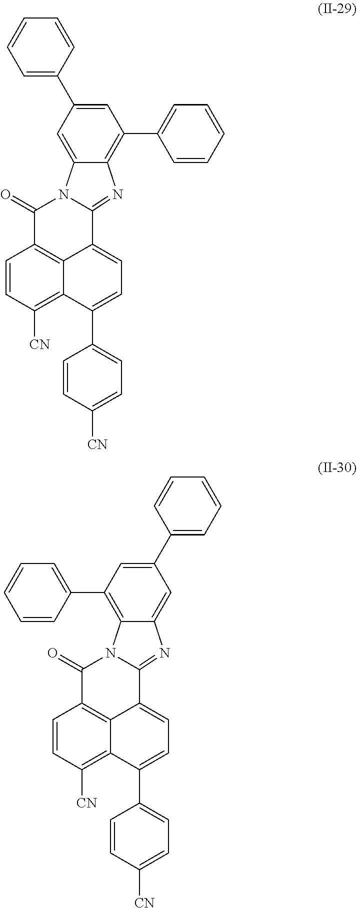 [SMILES: N#Cc1ccc(-c2ccc3c4c2c(C#N)ccc4c(=O)n2c4cc(-c5ccccc5)cc(-c5ccccc5)c4nc32)cc1 ; N#Cc1ccc(-c2ccc3c4c2c(C#N)ccc4c(=O)n2c3nc3cc(-c4ccccc4)cc(-c4ccccc4)c32)cc1]

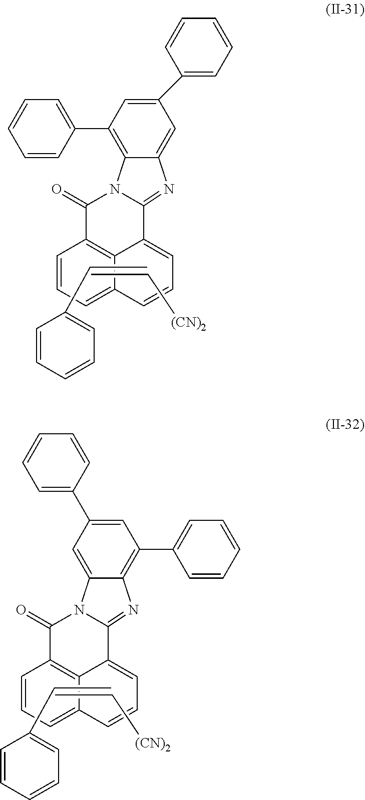

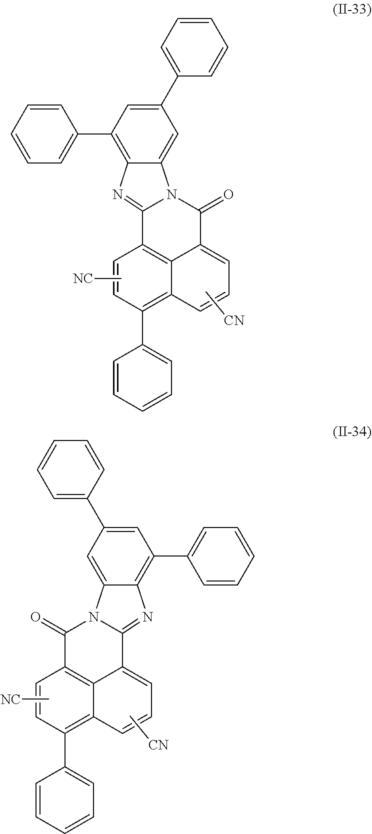

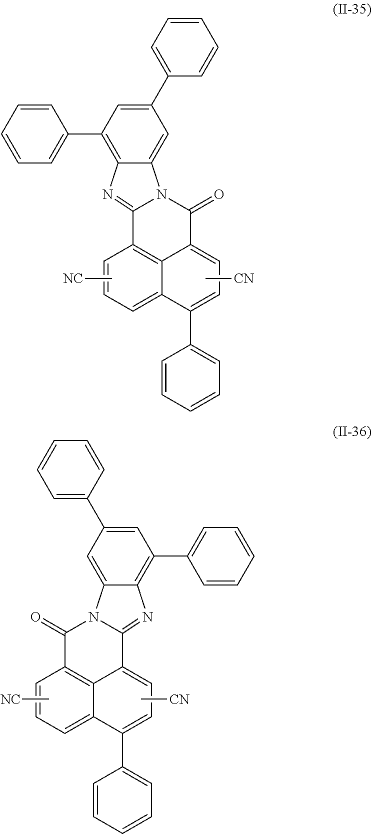

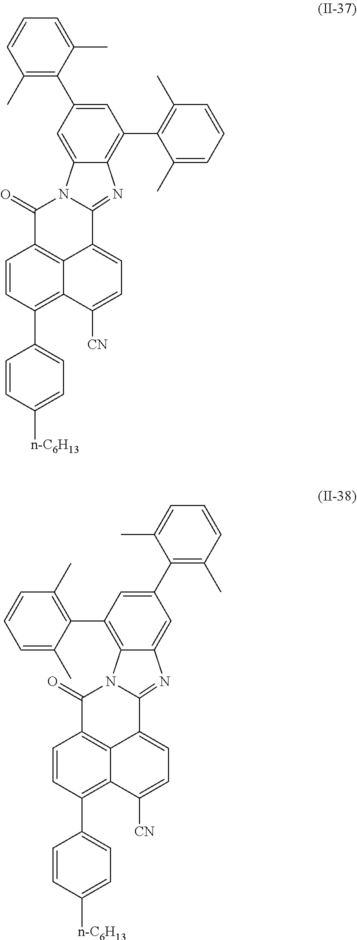

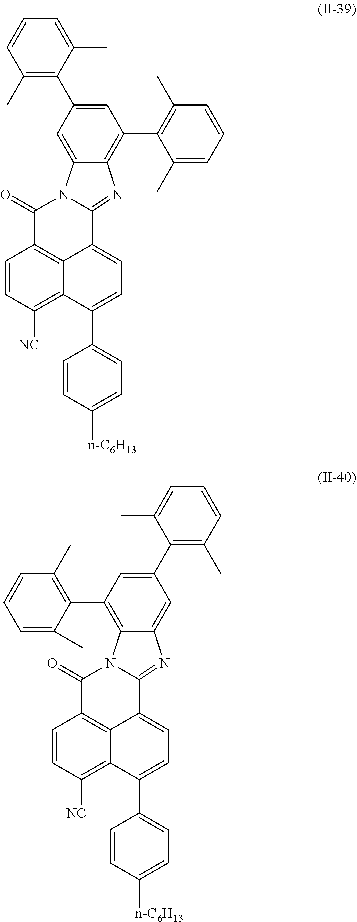

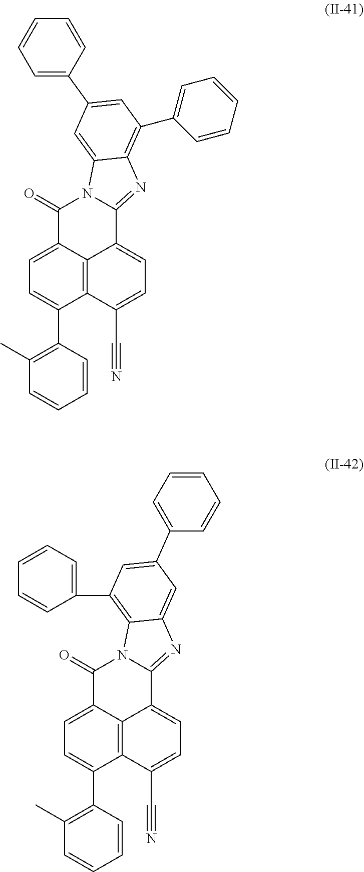 [SMILES: Cc1ccccc1-c1ccc2c(=O)n3c4cc(-c5ccccc5)cc(-c5ccccc5)c4nc3c3ccc(C#N)c1c23 ; Cc1ccccc1-c1ccc2c(=O)n3c(nc4cc(-c5ccccc5)cc(-c5ccccc5)c43)c3ccc(C#N)c1c23]

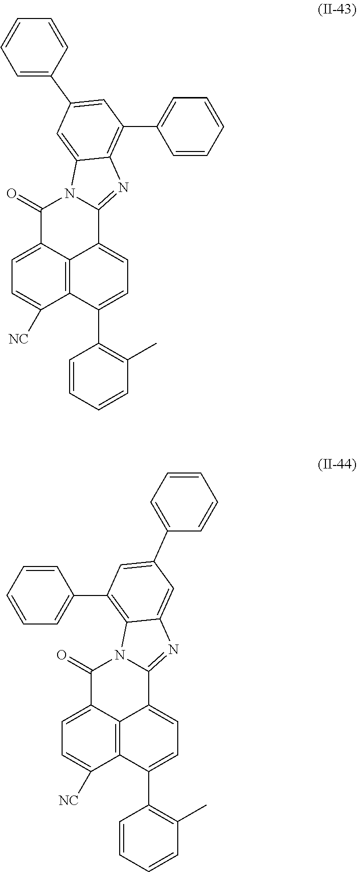 [SMILES: Cc1ccccc1-c1ccc2c3c1c(C#N)ccc3c(=O)n1c3cc(-c4ccccc4)cc(-c4ccccc4)c3nc21 ; Cc1ccccc1-c1ccc2c3c1c(C#N)ccc3c(=O)n1c2nc2cc(-c3ccccc3)cc(-c3ccccc3)c21]

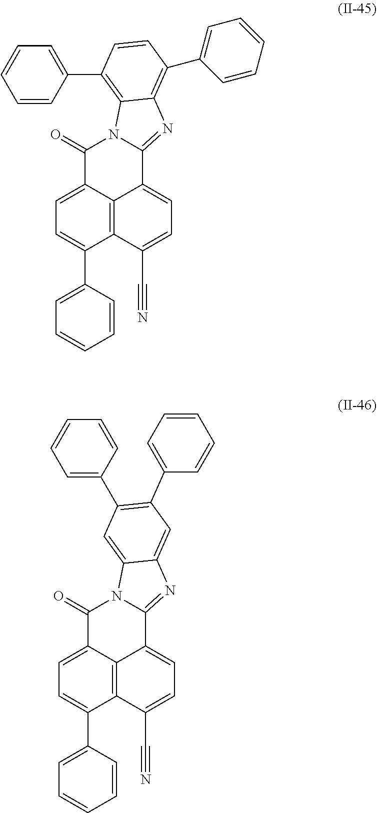 [SMILES: N#Cc1ccc2c3c1c(-c1ccccc1)ccc3c(=O)n1c2nc2c(-c3ccccc3)ccc(-c3ccccc3)c21 ; N#Cc1ccc2c3c1c(-c1ccccc1)ccc3c(=O)n1c3cc(-c4ccccc4)c(-c4ccccc4)cc3nc21]

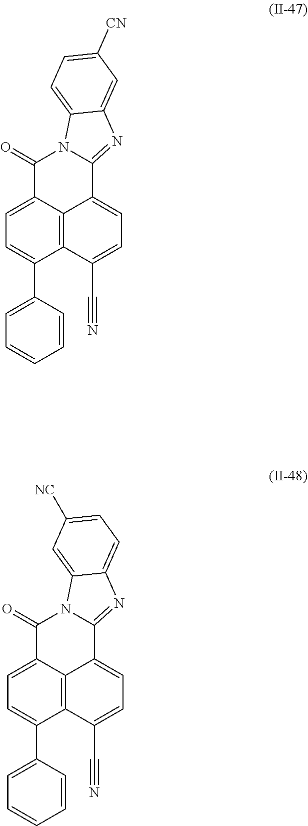 [SMILES: N#Cc1ccc2c(c1)nc1c3ccc(C#N)c4c(-c5ccccc5)ccc(c(=O)n21)c43 ; N#Cc1ccc2nc3c4ccc(C#N)c5c(-c6ccccc6)ccc(c(=O)n3c2c1)c54]

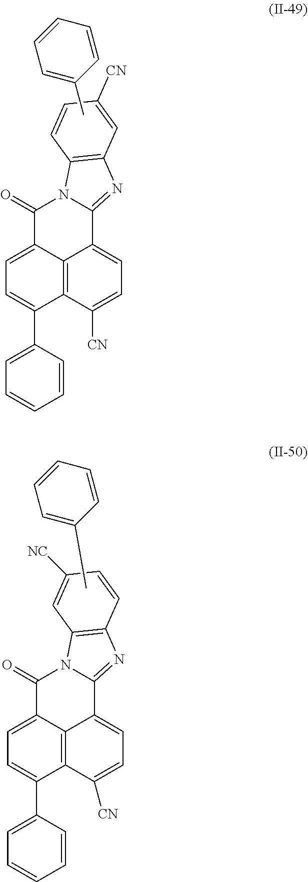

More especially preferred are compounds (II-11), (II-12), (II-13) and (II-14) and mixtures thereof.

### Organic Fluorescent Colorant (B2)

Compounds of formula (III) are known from WO 2015/169935. Compounds of formula (III) are usually green fluorescent dyes. With regard to the use in the converter of the present invention, the compound of formula (III) encompasses the following com-pounds of formulae (III-a) and (III-b) as well as compounds of formulae (III-c) and (IIII-d):

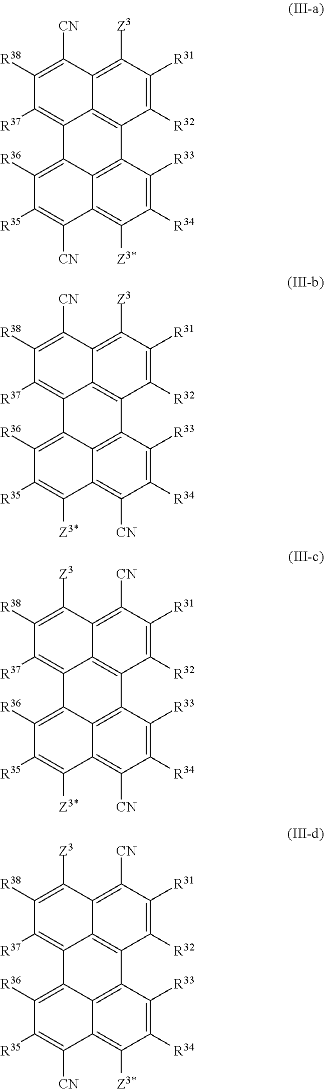

individually and mixtures thereof,
in which R³¹, R³², R³³, R³⁴, R³⁵, R³⁶, R³⁷, R³⁸, Z³ and Z³* are each as defined above.

In particular, preference is given to the compounds specified in WO 2015/169935 on page 12, line 9 to page 13, line 31. With regard to the use in the converter of the present invention, preferred are compounds of formula (III) selected from compounds of formulae (III-1), (III-2), (III-3), (III-4), (III-5), (III-6), (III-7), (III-8), (III-9), (III-10), (III-11), (III-12), (III-13), (III-14), (III-15), (III-16), (III-17), (III-18), (III-19), (III-20)

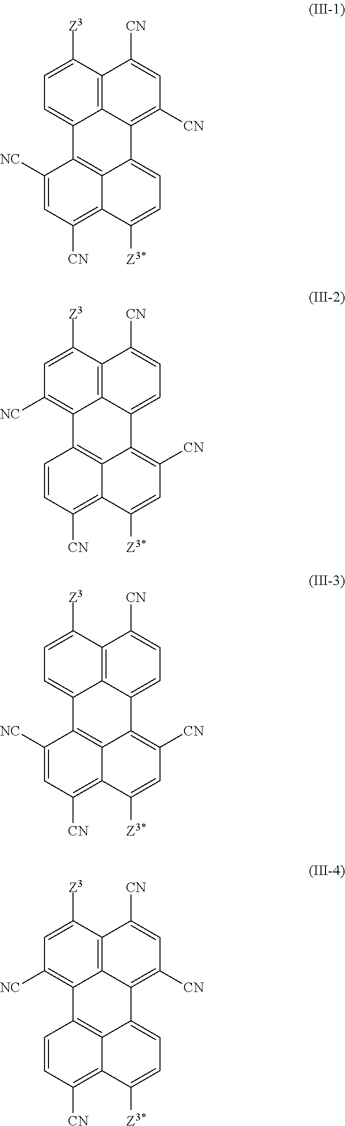

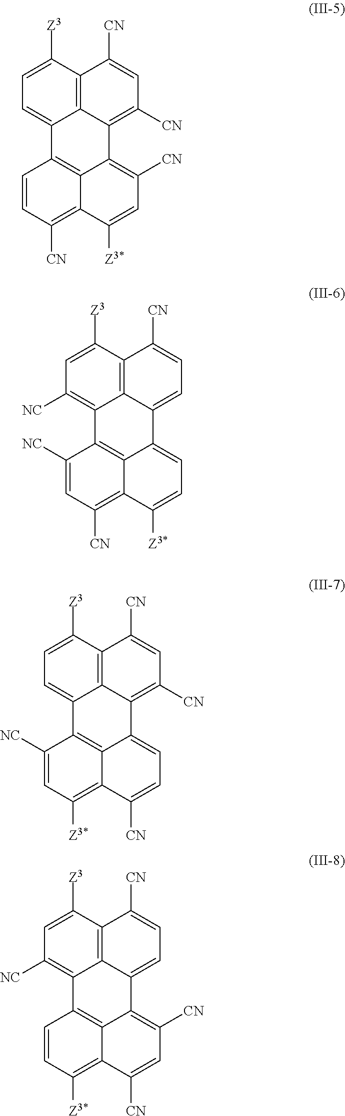

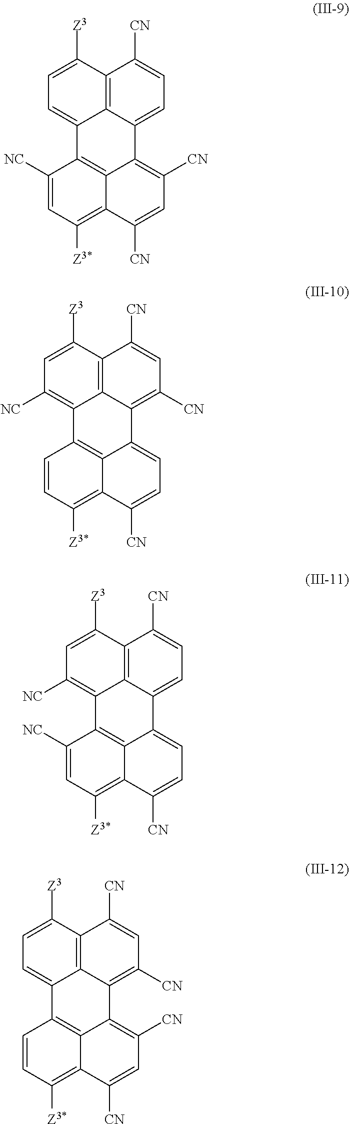

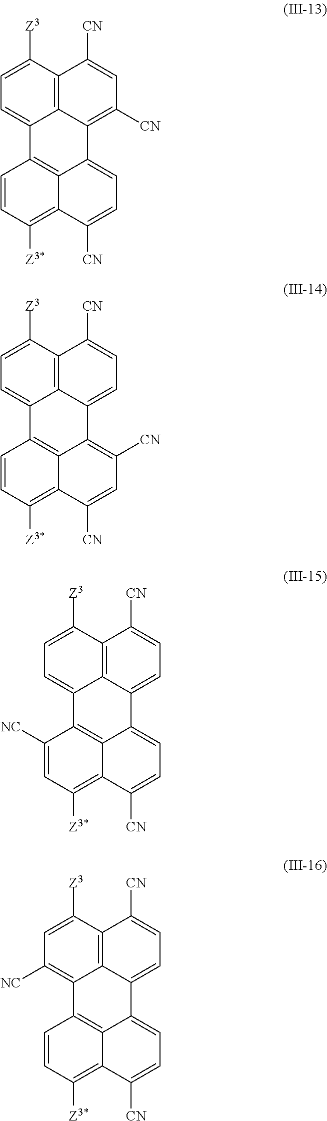

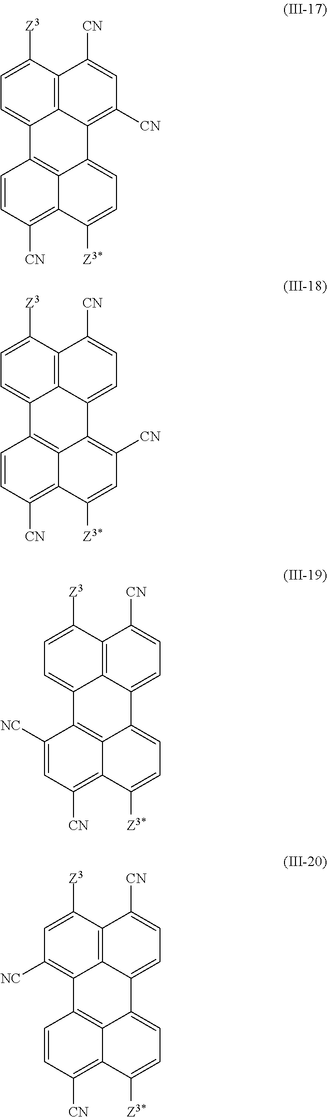

and mixtures thereof,
in which
Z³ is selected from C₁-C₆-alkyl, C₁-C₆-alkoxycarbonyl, phenyl, or phenyl bearing 1, 2 or 3 C₁-C₄-alkyl groups; and
Z³* is selected from C₁-C₆-alkyl, C₁-C₆-alkoxycarbonyl, phenyl, or phenyl bearing 1, 2 or 3 C₁-C₄-alkyl groups.

In a special embodiment, Z³* has the same meaning as Z³.

Among these, specific preference is given to perylene compounds of formulae (10.a), (10.b)

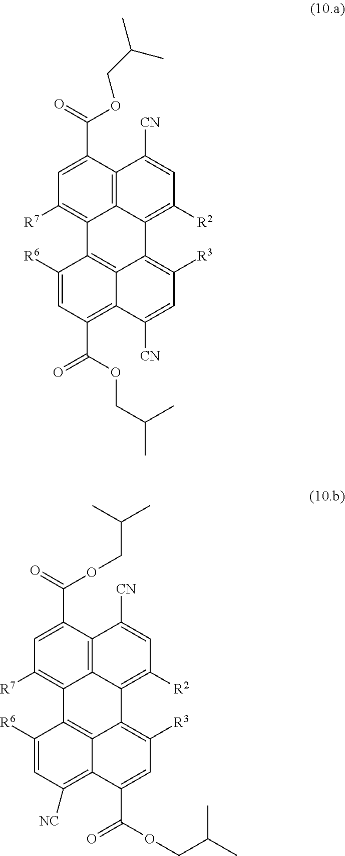

and mixtures of compounds of formulae (10.a) and (10.b),
in which
   three of the R², R³, R⁶ and R⁷ substituents are hydrogen; and
   one of the R², R³, R⁶ and R⁷ substituents is cyano.

### Organic Fluorescent Colorant (B3)

Cyanated compounds of formula (IV) are subject-matter of WO 2016/151068. Compounds of formula (IV) are usually green fluorescent dyes. With regard to the use in the converter of the present invention, the compound of formula (IV) is preferably a compound, wherein X⁴⁰ is O. Also preferred are compounds of formula (IV), wherein X⁴⁰ is S. Preference is given to the compounds specified in WO 2016/151068 on page 24, line 10 to page 34, line 4.

Among these, compounds of formula (IV) are especially preferred, wherein A is a radical of formula (A.2). Compounds of formula (IV), where A is a radical of formula (A.2) are also referred to as compounds of formula (IV-A.2),

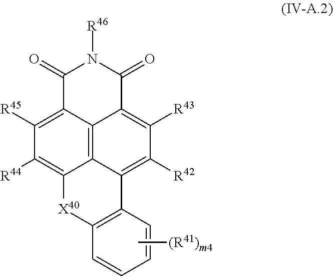

wherein
   m4, X⁴⁰, R⁴¹, R⁴², R⁴³, R⁴⁴, R⁴⁵ and R⁴⁶ are as defined above.

In the compounds of formula (I-A.2), R⁴⁶ is preferably selected from hydrogen, linear C₁-C₂₄-alkyl, branched C₃-C₂₄-alkyl, C₆-C₁₀-aryl and C₆-C₁₀-aryl-C₁-C₁₀-alkylene, where the aryl ring in the two last mentioned moieties is unsubstituted or substituted with 1, 2, 3, 4 or 5 identical or different radicals R^{46a}. Especially, R⁴⁶ is selected from linear C₁-C₂₄-alkyl, a radical of formula (B.1) and a radical of formula (B.2)

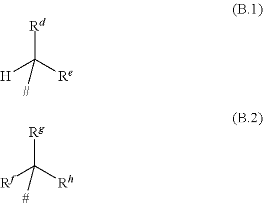

in which
# is the bonding site to the nitrogen atom;
R^{d} and R^{e}, in the formula (B.1), independently from each other are selected from C₁-C₂₃-alkyl, where the sum of the carbon atoms of the R^{d} and R^{e} radicals is an integer from 2 to 23;
R^{f}, R^{g} and R^{h}, in the formula (B.2) are independently selected from C₁— to C₂₀-alkyl, where the sum of the carbon atoms of the R^{f}, R^{g} and R^{h} radicals is an integer from 3 to 23.

Preferred radicals of formula (B.1) are: 1-methylethyl, 1-methylpropyl, 1-methylbutyl, 1-methylpentyl, 1-methylhexyl, 1-methylheptyl, 1-methyloctyl, 1-ethylpropyl, 1-ethylbutyl, 1-ethylpentyl, 1-ethylhexyl, 1-ethylheptyl, 1-ethyloctyl, 1-propylbutyl, 1-propylpentyl, 1-propylhexyl, 1-propylheptyl, 1-propyloctyl, 1-butylpentyl, 1-butylhexyl, 1-butylheptyl, 1-butyloctyl, 1-pentylhexyl, 1-pentylheptyl, 1-pentyloctyl, 1-hexylheptyl, 1-hexyloctyl, 1-heptyloctyl.

A particularly preferred radical of formula (B.2) is tert.-butyl.

Likewise especially, R⁴⁶ is a radical of formula (C.1), a radical of formula (C.2) or a radical of formula (C.3)

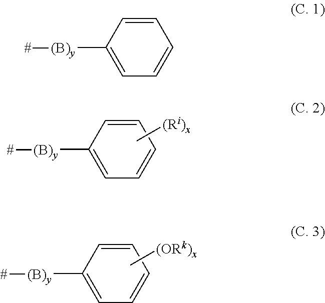

# represents the bonding side to the nitrogen atom;
B where present, is a C₁-C₁₀-alkylene group which may be interrupted by one or more nonadjacent groups selected from —O— and —S—;
y is 0 or 1,
Rⁱ is independently of one another selected from C₁-C₂₄-alkyl, C₁-C₂₄-fluoroalkyl, fluorine, chlorine or bromine;
R^{k} is independently of one another selected from C₁-C₂₄-alkyl;
x in formulae C.2 and C.3 is 1, 2, 3, 4 or 5.

Preferably, y is 0, i.e. the variable B is absent.

Irrespectively of its occurrence, Rⁱ is preferably selected from C₁-C₂₄-alkyl, more preferably linear C₁-C₁₀-alkyl or branched C₃-C₁₀-alkyl, especially isopropyl. Irrespectively of its occurrence, R^{k} is preferably selected from C₁-C₂₄-alkyl, more preferably linear C₁-C₁₀-alkyl or branched C₃-C₁₀-alkyl. The variable x in formulae C.2 and C.3 is preferably 1, 2 or 3.

A special group of embodiments relates to compounds of formula (IV-A.2), wherein the variables m4, X⁴⁰, R⁴¹, R⁴², R⁴³, R⁴⁴, and R⁴⁵ independently of each other or in particular in combination, have the following meanings:
X⁴⁰ is O or S;
R⁴² and R⁴⁴ are each cyano;
R⁴³ and R⁴⁵ are each hydrogen or one of R⁴³ and R⁴⁵ is bromine and the other of R⁴³ and R⁴⁵ is hydrogen;
R⁴¹ is selected from cyano, bromine, and phenyl which is unsubstituted or carries 1 or 2 radicals selected from C₁-C₄-alkyl;
R⁴⁶ is selected from hydrogen, C₁-C₂₄-linear alkyl, branched C₃-C₂₄-alkyl, a radical of formula (C.1), a radical of formula (C.2) and a radical of formula (C.3);
m4 is 0 or 1.

Even more preferably,
X⁴⁰ is O or S;
R⁴² and R⁴⁴ are each cyano;
R⁴³ and R⁴⁵ are each hydrogen;
R⁴¹ is selected from cyano, bromine or phenyl which is unsubstituted or carries 1 or 2 radicals selected from C₁-C₄-alkyl; especially cyano;
R⁴⁶ is selected from linear C₁-C₂₄-alkyl, branched C₃-C₂₄-alkyl, a radical of formula (C.1), a radical of formula (C.2) or a radical of formula (C.3); especially linear C₁-C₂₄-alkyl, branched C₃-C₂₄-alkyl, or phenyl which carries 1 or 2 radicals selected from C₁-C₄-alkyl such as 2,6-diisopropylphenyl;
m4 is 0 or 1.

Examples for preferred compounds of formula (IV-A.2) are shown below:

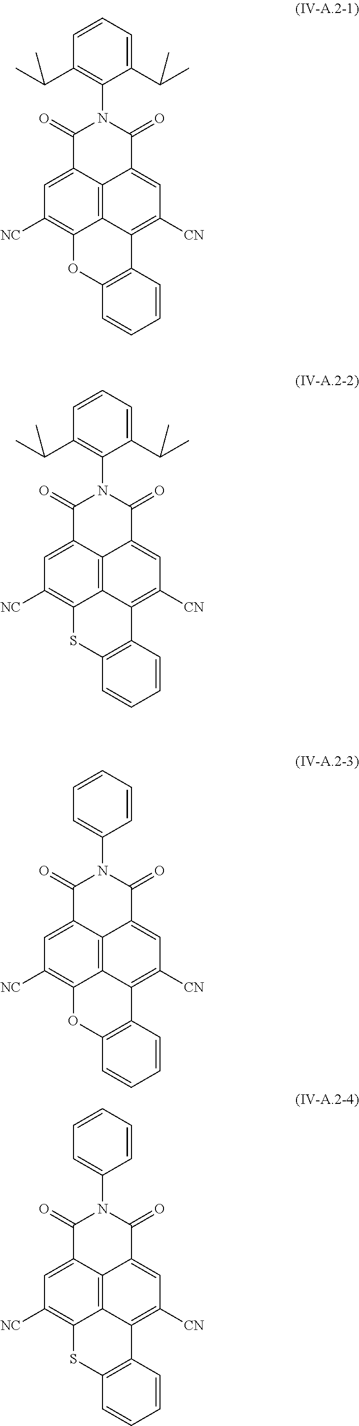

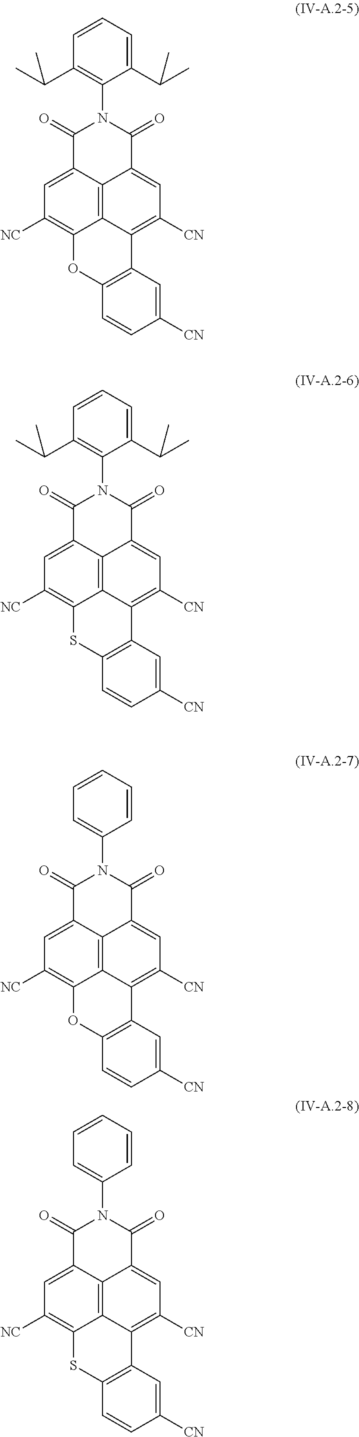

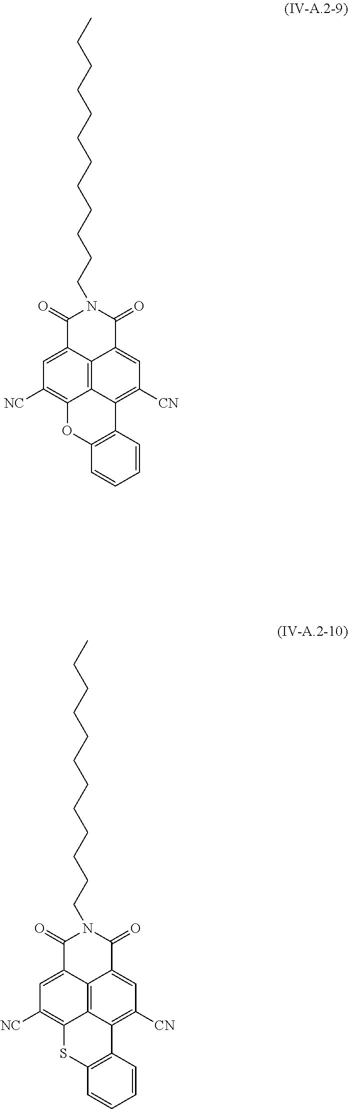 [SMILES: CCCCCCCCCCCCn1c(=O)c2cc(C#N)c3oc4ccccc4c4c(C#N)cc(c1=O)c2c34 ; CCCCCCCCCCCCn1c(=O)c2cc(C#N)c3sc4ccccc4c4c(C#N)cc(c1=O)c2c34]

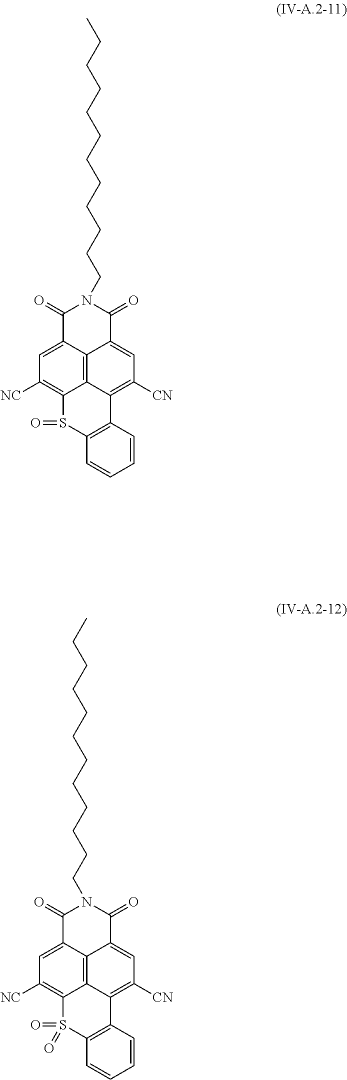 [SMILES: CCCCCCCCCCCCN1C(=O)c2cc(C#N)c3c4c(c(C#N)cc(c24)C1=O)S(=O)c1ccccc1-3 ; CCCCCCCCCCCCN1C(=O)c2cc(C#N)c3c4c(c(C#N)cc(c24)C1=O)S(=O)(=O)c1ccccc1-3]

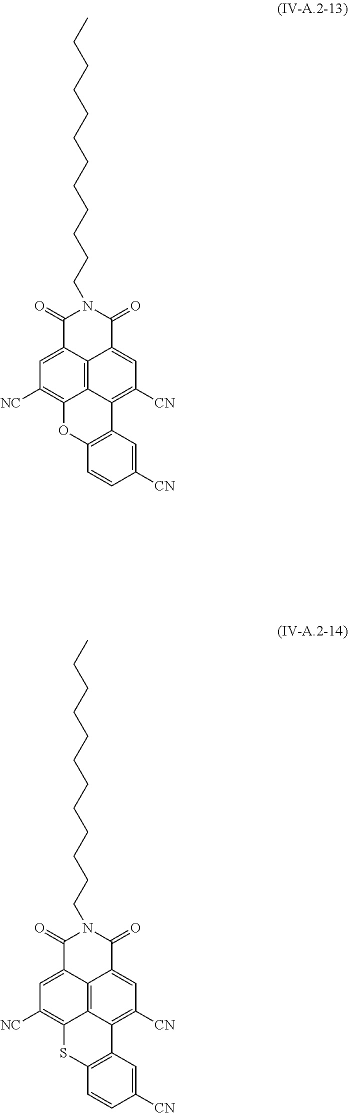 [SMILES: CCCCCCCCCCCCn1c(=O)c2cc(C#N)c3oc4ccc(C#N)cc4c4c(C#N)cc(c1=O)c2c34 ; CCCCCCCCCCCCn1c(=O)c2cc(C#N)c3sc4ccc(C#N)cc4c4c(C#N)cc(c1=O)c2c34]

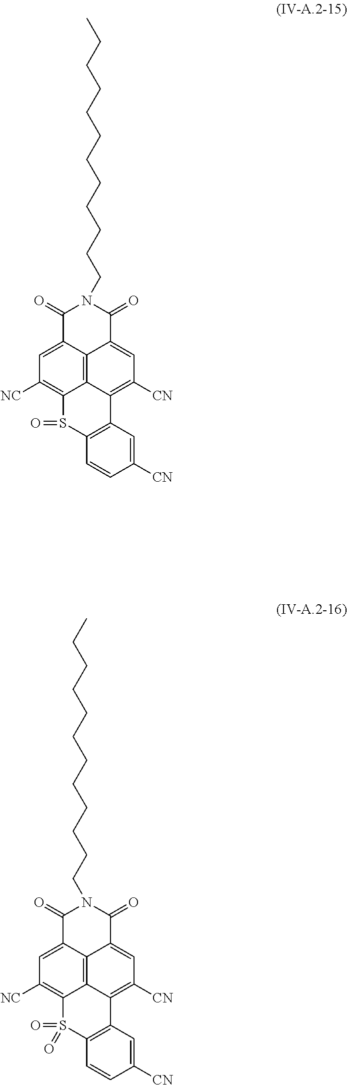 [SMILES: CCCCCCCCCCCCN1C(=O)c2cc(C#N)c3c4c(c(C#N)cc(c24)C1=O)S(=O)c1ccc(C#N)cc1-3 ; CCCCCCCCCCCCN1C(=O)c2cc(C#N)c3c4c(c(C#N)cc(c24)C1=O)S(=O)(=O)c1ccc(C#N)cc1-3]

In particular, organic fluorescent colorant (B4) is selected from compounds IV-A.2-1, IV-A.2-6 or IV-A.2-9.

### ORGANIC Fluorescent Colorant (B4)

Benzoxanthene compounds of formula (V) are known from WO 2014/131628. They are usually green fluorescent dyes. Benzothioxanthene compounds of formula (V) are known for example from U.S. Pat. No. 3,357,985.

Preferred are benzoxanthene compounds of formula (V), wherein R⁵¹ is phenyl which is unsubstituted or carries 1 or 2 substituents selected from C₁-C₁₀-alkyl, R⁵³ and R⁵⁴ are each phenyl and R⁵², R⁵⁵, R⁵⁶, R⁵⁷, R⁵⁸ and R⁵⁹ are each hydrogen. Suitable compounds are depicted in FIG. 2A, FIG. 2B and FIG. 2C of WO 2014/131628. Preferred are also benz(othi)oxanthene compounds of formula (V), wherein X⁵ is O or S, R⁵¹ is C₁-C₂₄-alkyl and R⁵² to R⁵⁹ are hydrogen. Preferably, R⁵¹ is C₆-C₂₀-alkyl. Suitable compounds are depicted below

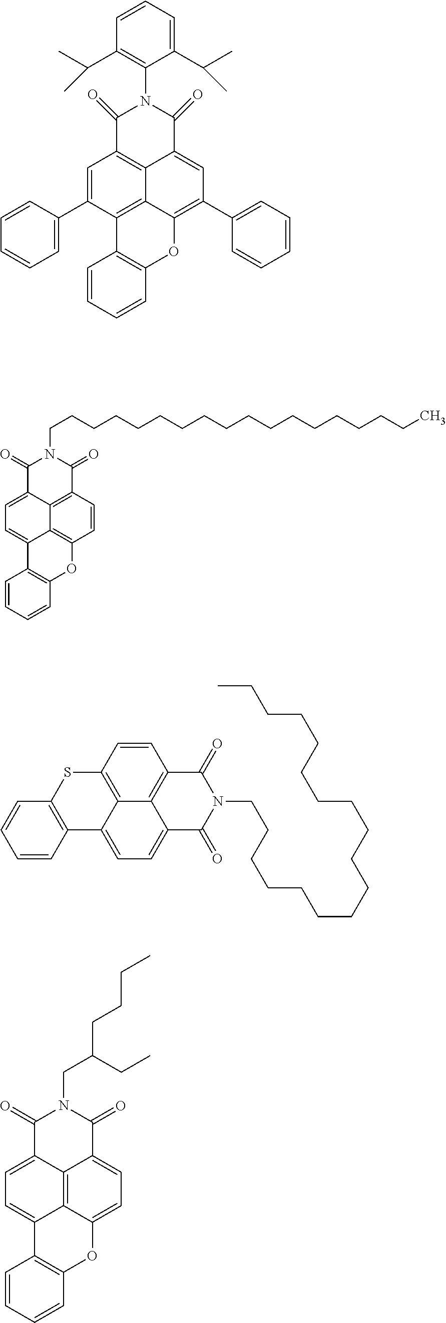 [SMILES: CC(C)c1cccc(C(C)C)c1-n1c(=O)c2cc(-c3ccccc3)c3oc4ccccc4c4c(-c5ccccc5)cc(c1=O)c2c34 ; CCCCCCCCCCCCCCCCCCn1c(=O)c2ccc3oc4ccccc4c4ccc(c1=O)c2c34 ; CCCCCCCCCCCCCCCCCCn1c(=O)c2ccc3sc4ccccc4c4ccc(c1=O)c2c34 ; CCCCC(CC)Cn1c(=O)c2ccc3oc4ccccc4c4ccc(c1=O)c2c34]

### Organic Fluorescent Colorant (B5)

Benzimidazoxanthenisoquinoline compounds of formula (VIA) and (VIB) are known from WO 2015/062916. In general, they are green fluorescent dyes. Suitable compounds are depicted at page 3, line 24 to page 8, line 24, especially FIG. 3A, FIG. 3B, FIG. 3C of WO 2015/062916.

### Organic Fluorescent Colorant (B6)

Compounds having a structural unit of formula (VII) are known from WO 2012/168395. In general, they are green fluorescent dyes. With regard to the use in the converter of the present invention, the compound having a structural unit of formula (VII) is preferably a compound as specified in WO 2012/168395, at page 28, line 14 to page 32, line 5.

With regard to the use in the converter of the present invention, the compound having a structural unit of formula (VII) is more preferably selected from compounds of formulae (VII-1), (VII-2), (VII-3), (VII-4), (VII-5), (VII-6), (VII-7), (VII-8), (VII-9), (VII-10), (VII-11), (VII-12), (VII-13), (VII-14), (VII-15), (VII-16), (VII-17), (VII-18), (VII-19), (VII-20), (VII-21), (VII-22), (VII-23), (VII-24), (VII-25), (VII-26), (VII-27), (VII-28), (VII-29), (VII-30), (VII-31), (VII-32), (VII-33), (VII-34), (VII-35), (VII-36), (VII-37), (VII-38), (VII-39), (VII-40), (VII-41), (VII-42), (VII-43), (VII-44), (VII-45), (VII-46), (VII-47), (VII-48), (VII-49), (VII-50), (VII-51), (VII-52), (VII-53), (VII-54), (VII-55), or mixtures thereof

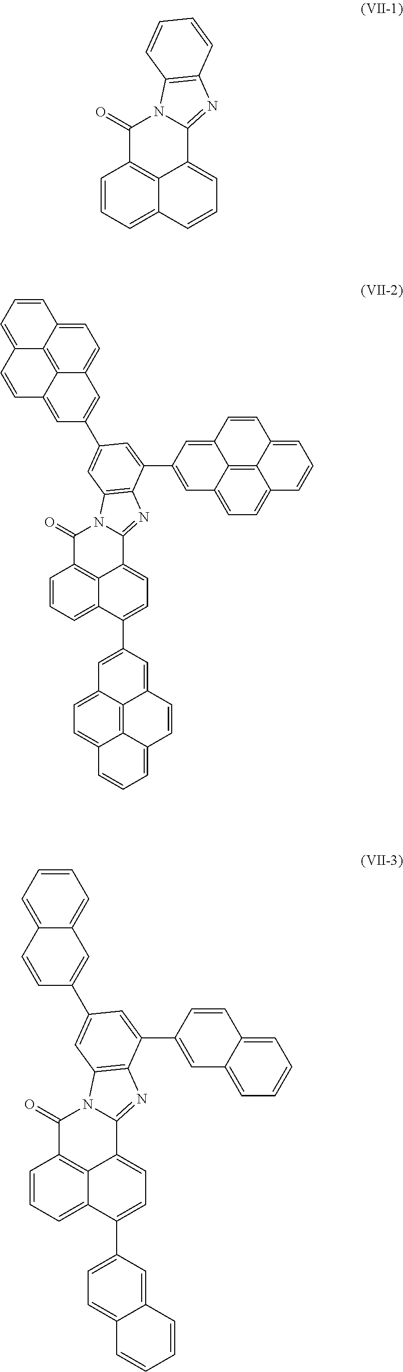

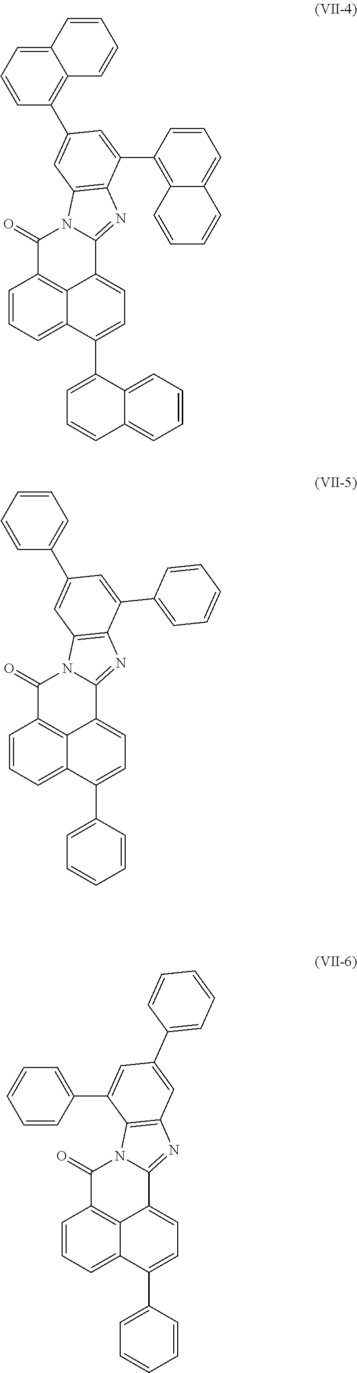

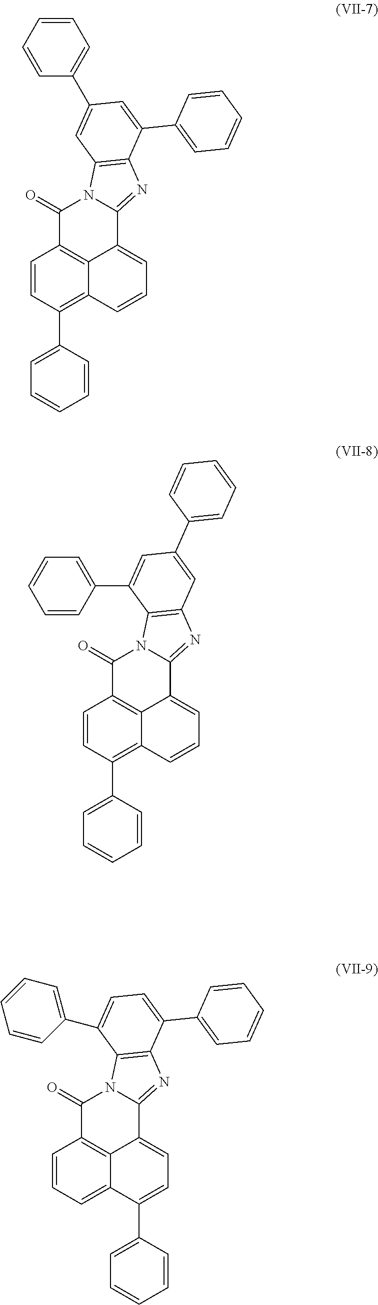

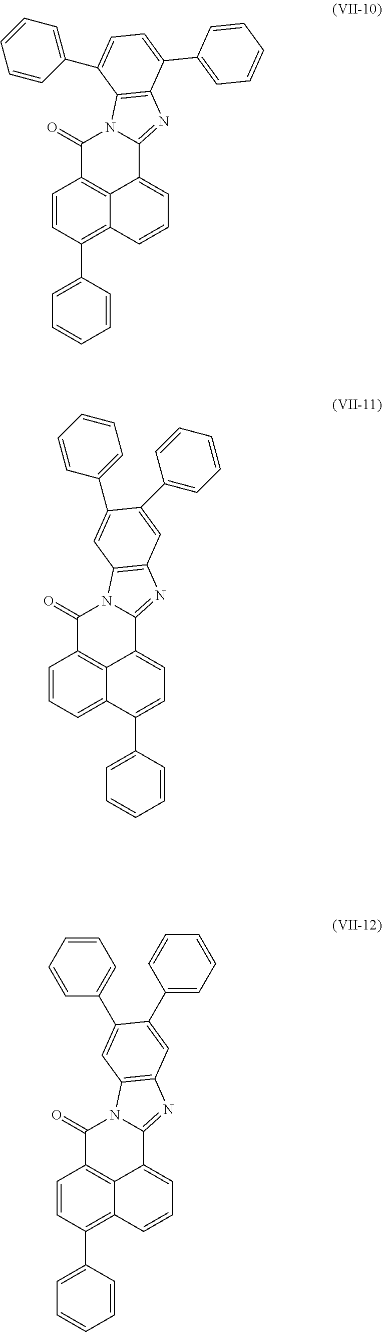

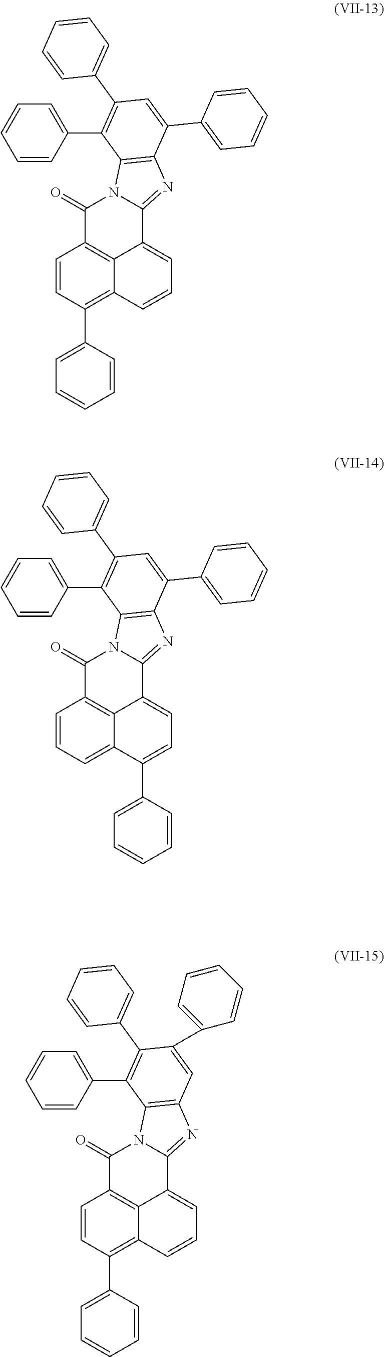

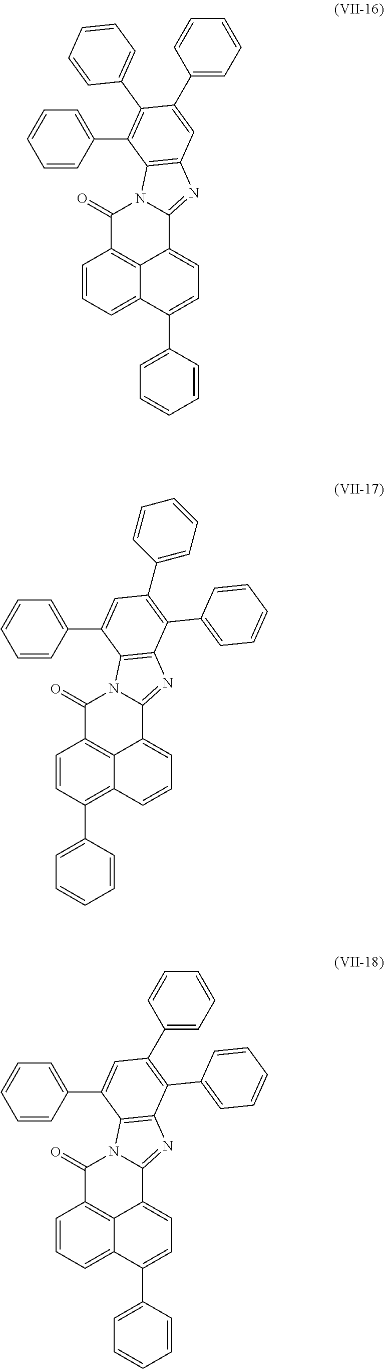

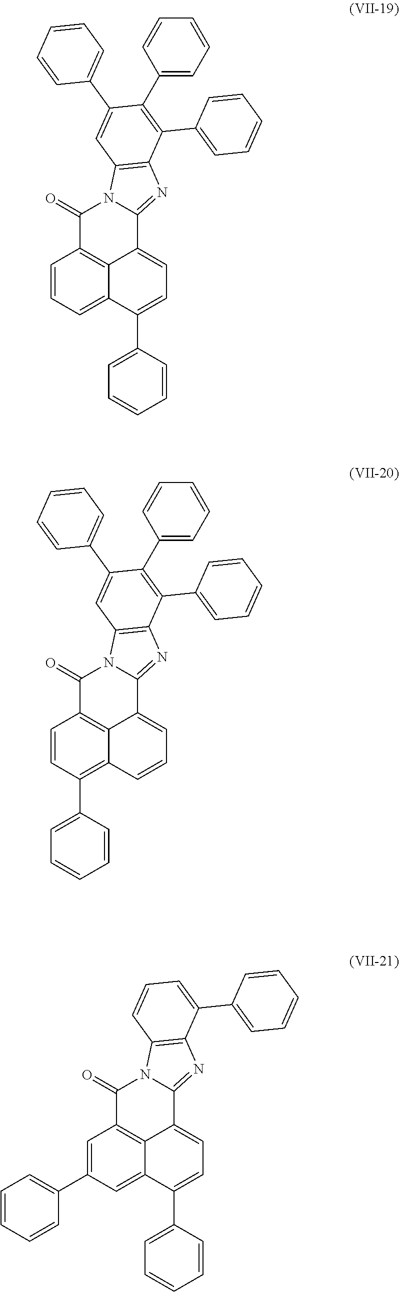

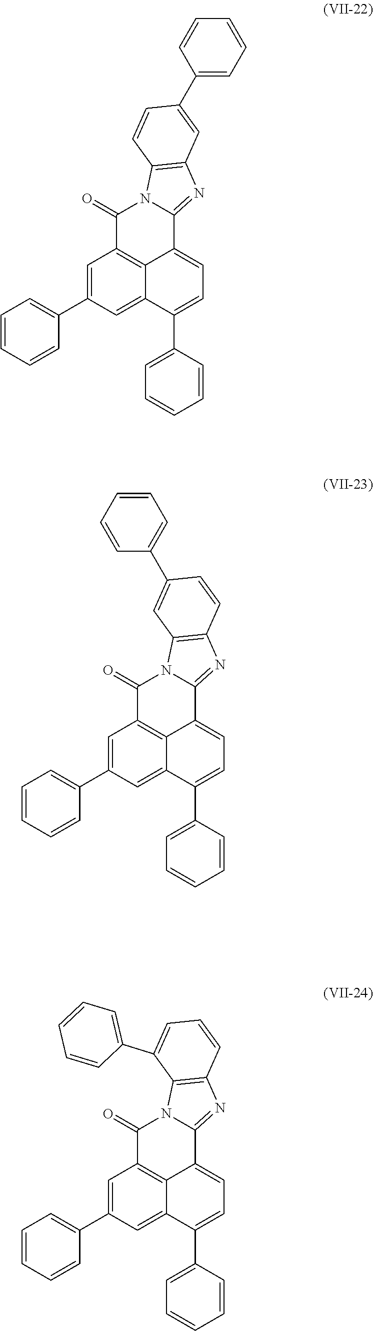

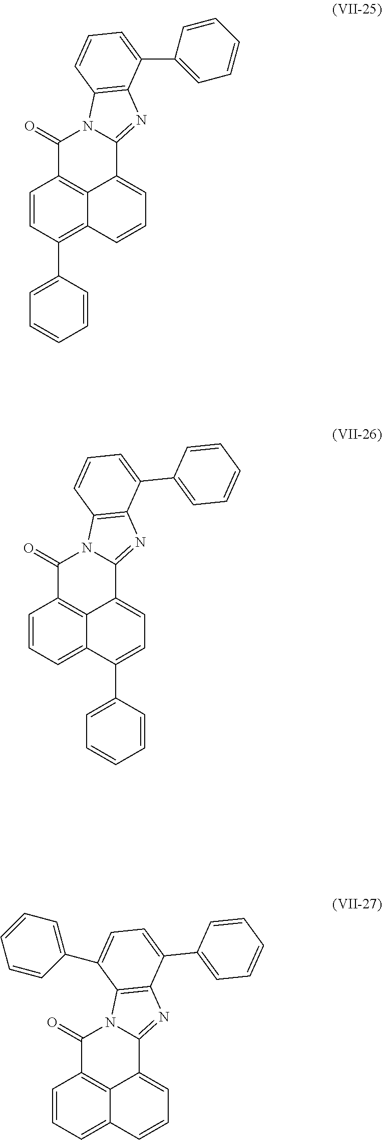

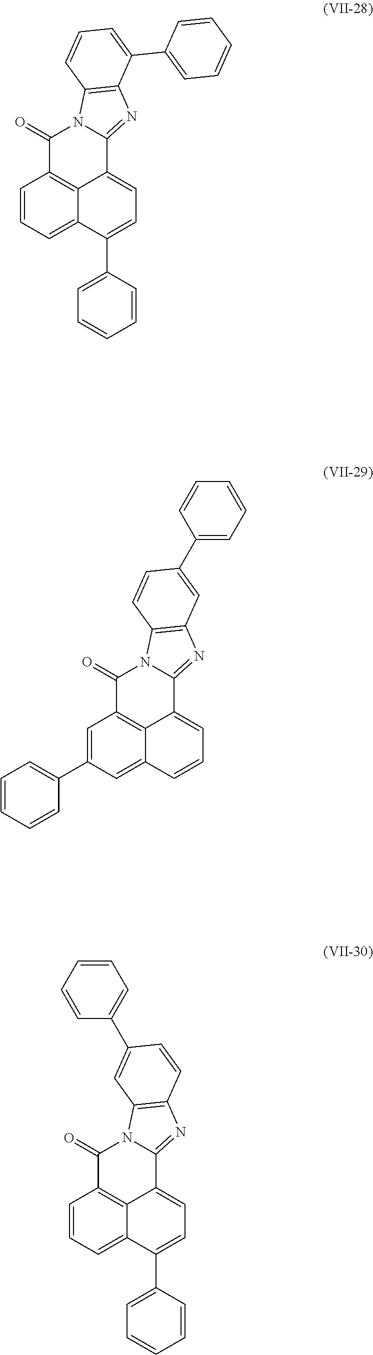

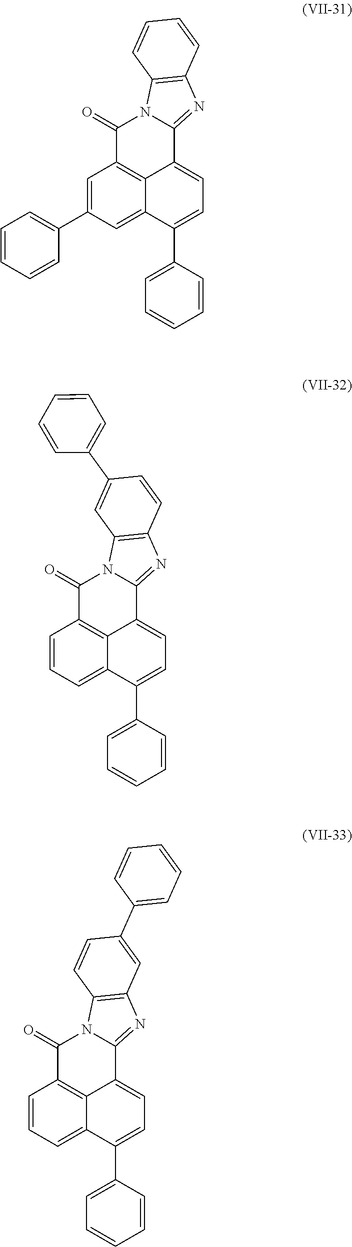

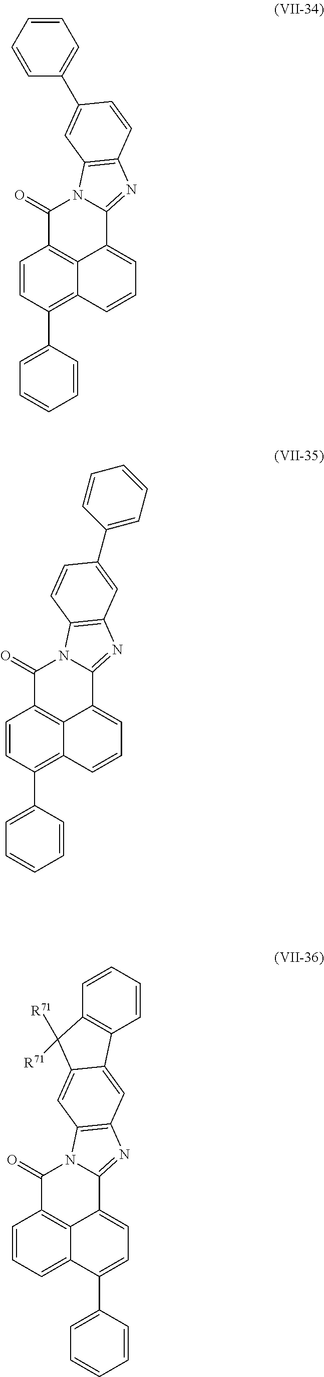

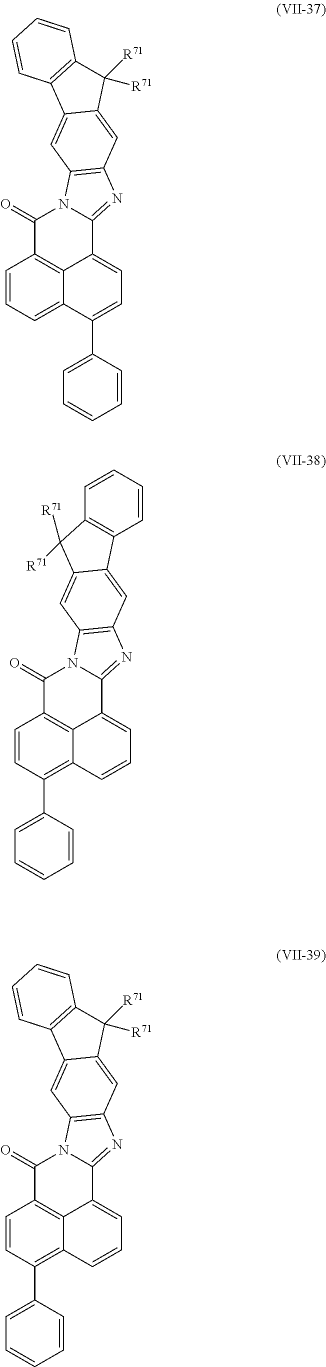

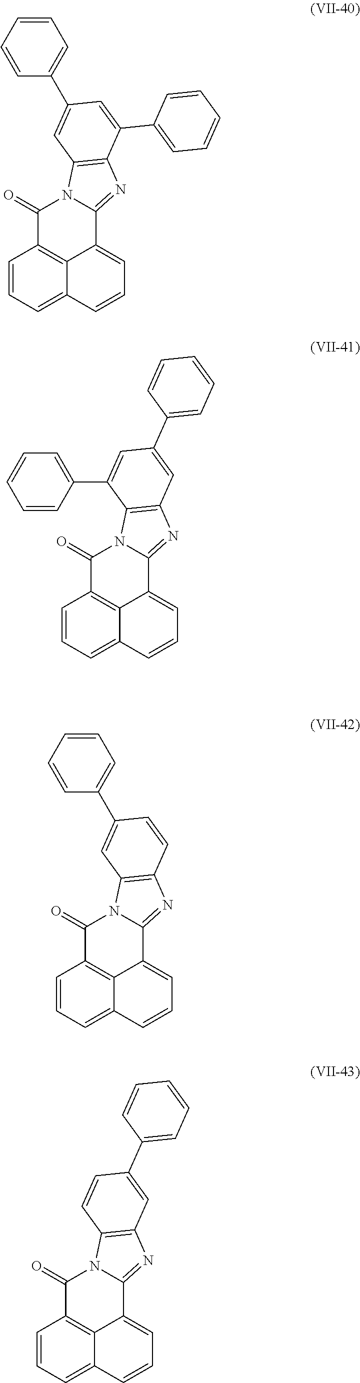

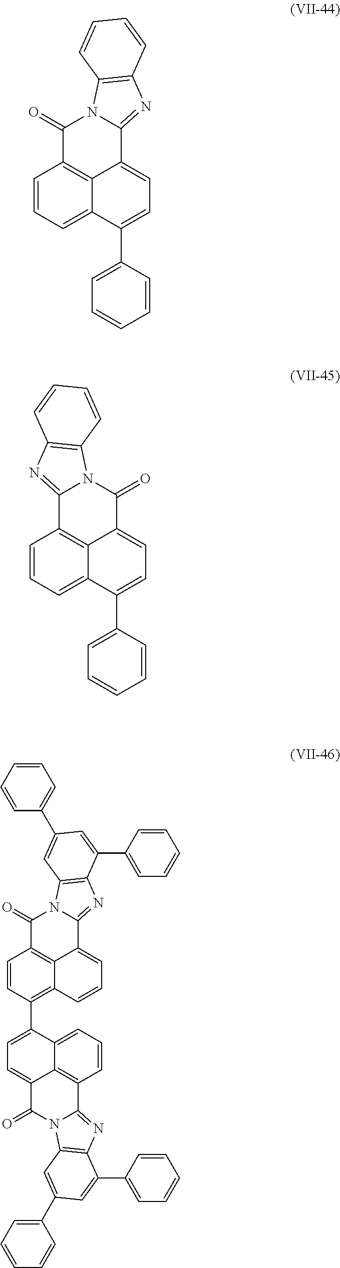

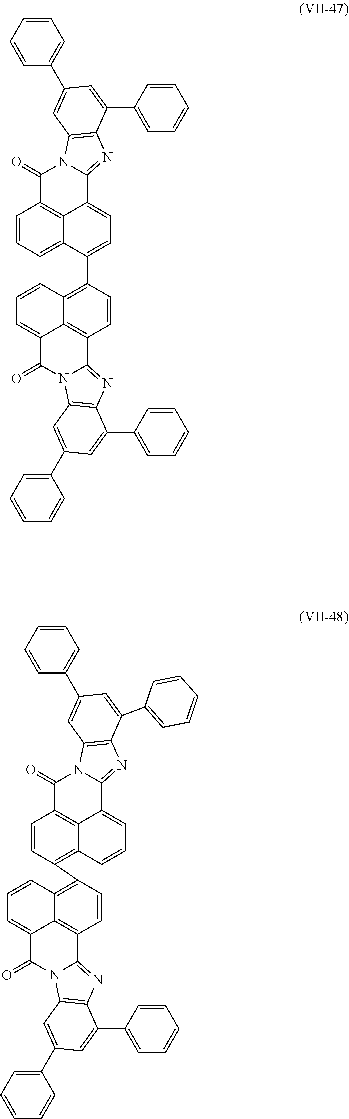

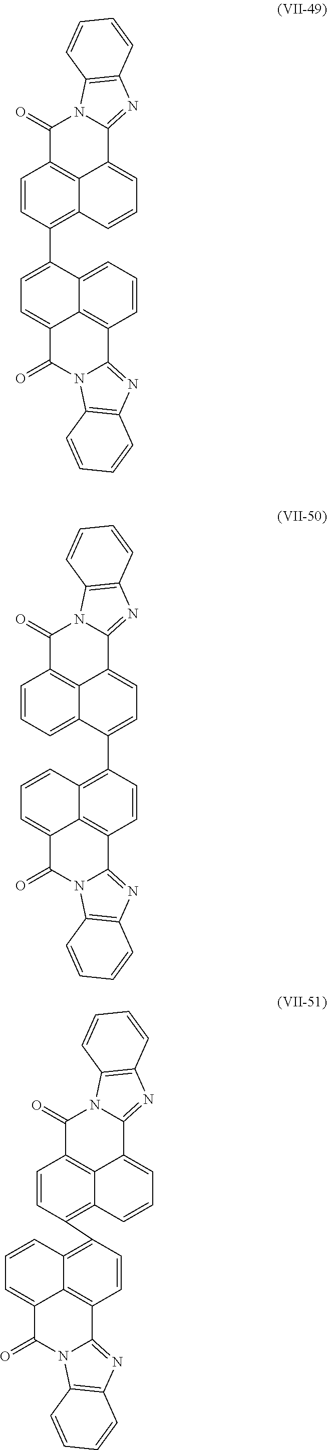

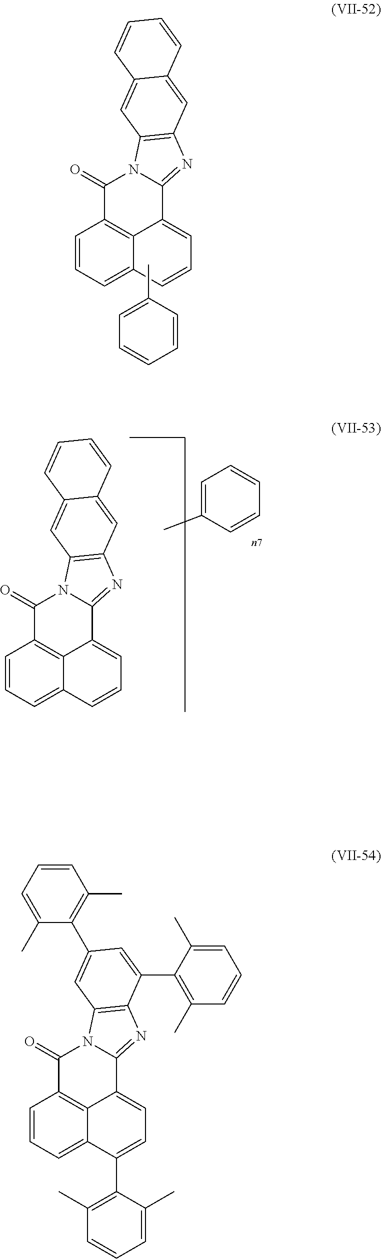

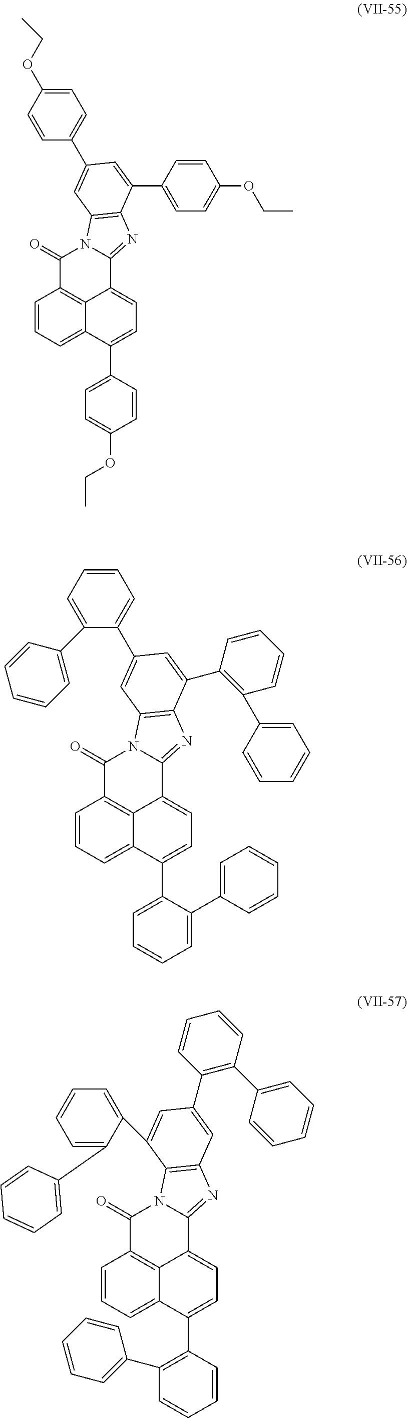

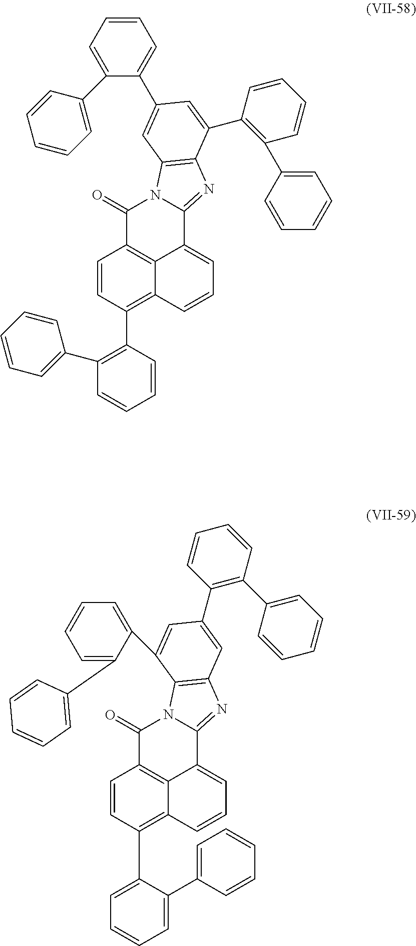

where
n7 is a number from 0, 1, 2, 3, 4, 5, 6, 7, 8, 9 or 10;
R⁷¹ is independently hydrogen, C₁-C₁₈-alkyl or cycloalkyl, the carbon chain of which may comprise one or more —O—, —S—, —CO—, —SO— and/or —SO₂— moieties and which may be mono- or polysubstituted;
   aryl or heteroaryl which may be mono- or polysubstituted.

Especially preferred are the compounds of formulae (VII-5), (VII-6), (VII-7) and (VII-8) and mixtures thereof. More especially preferred are the compounds of formulae (VII-5), (VII-7) or a mixture of the compounds of formulae (VII-5) and (VII-7). Especially preferred are also the compounds of formulae (VII-56), (VII-57), (VII-58) and (VII-59) and mixtures thereof.

### Organic Fluorescent Colorant (B7)

Perylene imide compounds of formula (VIII) and (IX) are well known in the art, e.g. from WO 2007/006717 or U.S. Pat. No. 6,472,050. 9-Cyano substituted perylene-3,4-dicarboxylic acid monoimides of formula (IX) are also known from WO 2004/029028. They are usually green fluorescent dyes.

Preferably, in compounds of formula (VIII), R⁸¹ and R⁸² are a linear or branched C₁-C₁₈ alkyl radical, a C₄-C₈ cycloalkyl radical which may be mono- or polysubstituted by halogen or by linear or branched C₁-C₁₈ alkyl, or phenyl or naphthyl which may be mono- or polysubstituted by halogen or by linear or branched C₁-C₁₈ alkyl. Preferably, R⁸¹ and R⁸² have the same meaning.

In one embodiment, R⁸¹ and R⁸² in formula VIII represent compounds with what is called swallowtail substitution, as specified in WO 2009/037283 A1 at page 16, line 19 to page 25, line 8. In a preferred embodiment, R⁸¹ and R⁸², independently of each other, are a 1-alkylalkyl, for example 1-ethylpropyl, 1-propylbutyl, 1-butylpentyl, 1-pentylhexyl or 1-hexylheptyl.

In a preferred embodiment, the organic fluorescent colorant (B7) is selected from compounds of formula (VIII-1)

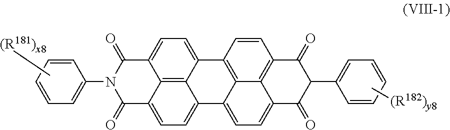

wherein
x8 is 1, 2 or 3;
y8 is 1, 2 or 3;
R¹⁸¹ is C₁-C₄-alkyl; and
R¹⁸² is C₁-C₄-alkyl.

Preferably, x8 is 2. Preferably, y8 is 2. Preferably, R¹⁸¹ and R¹⁸² are selected from isopropyl and tert-butyl.

A preferred compound of formula (VIII) is N,N′-bis(2,6-diisopropylphenyl)-3,4,9,10-perylenetetracarboxylic diimide (CAS-number: 82953-57-9).

Suitable 9-cyano substituted perylene-3,4-dicarboxylic acid monoimides of formula (IX) are preferably those, wherein R⁹² is a linear or branched C₁-C₁₈ alkyl radical, a C₄-C₈ cycloalkyl radical which may be mono- or polysubstituted by halogen or by linear or branched C₁-C₁₈ alkyl, or phenyl or naphthyl which may be mono- or polysubstituted by halogen or by linear or branched C₁-C₁₈ alkyl.

In one embodiment, R⁹² in formula IX represents compounds with what is called swallowtail substitution, as specified in WO 2009/037283 A1 at page 16, line 19 to page 25, line 8. In a preferred embodiment, R⁹², is a 1-alkylalkyl, for example 1-ethylpropyl, 1-propylbutyl, 1-butylpentyl, 1-pentylhexyl or 1-hexylheptyl. In another preferred embodiment, R⁹² is 2,4-di(tert-butyl)phenyl 2,6-diisopropylphenyl or 2,6-di(tert-butyl)phenyl. In particular, R⁹² is 2,6-diisopropylphenyl.

### Organic Fluorescent Colorant (B8)

4-Amino-substituted naphthalimide compounds of formula (X) are known in the art. They are usually green fluorescent compounds. Suitable 4-amino substituted naphthalimide compounds of formula (X) are preferably those, wherein R¹⁰¹ is linear or branched C₁-C₁₀-alkyl, C₂-C₁₀-alkyl which is interrupted by one or more oxygen, or C₃-C₅-cycloalkyl. R¹⁰² is preferably hydrogen. A suitable compound of formula (X) is 4-(butylamino)-N-butyl-1,8-naphthalimide (CAS Number: 19125-99-6). Likewise preferably, R¹⁰² is linear or branched C₁-C₁₀-alkyl. The compounds of formula (X) can be synthesized in two steps. The first step may be the condensation of 4-chloro-1,8-naph-thalic anhydride with amines in a solvent, such as 1,4-dioxane or 2-methoxyethanol under reflux yielding the corresponding 4-chloro-1,8-naphthalimides. The second step in-volves the substitution of the chlorine atom with aliphatic primary or secondary amines.

### Organic Fluorescent Colorant (B9)

7-(Diethylamino)-3-(5-methylbenzo[d]oxazol-2-yl)-2H-chromen-2-one is also known as Disperse Yellow (CAS Registry no. 34564-13-1). It is a green fluorescent compound

### Organic Fluorescent Colorant (B10)

Compounds of formulae (XIA) and (XIB) are known from U.S. Pat. No. 5,470,502. They are usually green fluorescent dyes. Preferred are compounds of formulae (XIA) and (XIB), wherein R¹¹¹ is linear C₁-C₁₀-alkyl or branched C₃-C₁₀-alkyl. Preferred examples are diisobutyl-3,9-perylenedicarboxylate, diisobutyl-3,10-perylenedicarboxylate and mixtures thereof. Especially preferred is a mixture of diisobutyl-3,9-perylenedicarboxylate and diisobutyl-3,10-perylenedicarboxylate.

### Organic Fluorescent Colorant (B111)

Compounds of formulae (XIIA) and (XIIB) are known from U.S. Pat. No. 5,470,502. They are usually green fluorescent dyes. Preferred are compounds of formulae (XIIA) and (XIIB), wherein R¹²¹ is linear C₁-C₁₀-alkyl or branched C₃-C₁₀-alkyl. Preferred examples are diisobutyl-4,10-dicyanoperylene-3,9-dicarboxylate and diisobutyl-4,9-dicyanop-erylene-3,10-dicarboxylate and mixtures thereof. Especially preferred is a mixture of diisobutyl-4,10-dicyanoperylene-3,9-dicarboxylate and diisobutyl-4,9-dicyanoperylene-3,10-dicarboxylate.

### Organic Fluorescent Colorant (B12)

Naphthoylbenzimidazole compounds of formula (1) are known from WO 2018/134261. Compounds of formula (XIII) are usually green fluorescent com-pounds.

With regard to the use in converters, compounds of formula (XIII) are preferred which correspond to a compound of formula (XIII-A)

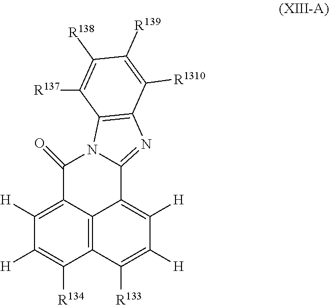

wherein
R¹³³ and R¹³⁴ are each independently hydrogen, phenyl, phenyl which carries 1 or 2 cyano groups or phenyl which carries 1, 2 or 3 substituents selected from C₁-C₁₀-alkyl; and
R¹³⁷, R¹³⁸, R¹³⁹ and R¹³¹⁰ are each independently hydrogen, phenyl, phenyl which carries 1 or 2 cyano groups or phenyl which carries 1, 2 or 3 substituents selected from C₁-C₁₀-alkyl.

Among the compounds of formula (XIII-A), preference is given to compounds, in which R¹³⁸ and R¹³¹⁰ have the same meaning. Likewise, preference is given to com-pounds, in which R¹³⁷ and R¹³⁹ have the same meaning. In particular, R¹³⁸ and R¹³¹⁰ have the same meaning and R¹³⁷ and R¹³⁹ have the same meaning. In particular, R¹³⁷ and R¹³⁹ have the same meaning and are hydrogen.

A particular preferred embodiment of the invention relates to compounds of formula (XIII-A), wherein
R¹³³ and R¹³⁴ are each independently selected from hydrogen, phenyl, phenyl which carries 1 or 2 cyano groups and phenyl which carries 1, 2 or 3 C₁-C₁₀-alkyl substituents; in particular hydrogen, phenyl or phenyl which carries 1 cyano group;
R¹³⁷ is hydrogen;
R¹³⁸ is phenyl, phenyl which carries 1 or 2 cyano groups or phenyl which carries 1, 2 or 3 C₁-C₁₀-alkyl substituents;
R¹³⁹ is hydrogen; and
R¹³¹⁰ is phenyl, phenyl which carries 1 or 2 cyano groups or phenyl which carries 1, 2 or 3 C₁-C₁₀-alkyl substituents.

In particular, R¹³⁸ is 4-cyanophenyl. In particular, R¹³¹⁰ is 4-cyanophenyl. Even more preferred are compounds of formula (XIII-A), wherein
R¹³³ is phenyl, phenyl which carries 1 cyano group; or phenyl which carries 1 substituent selected from C₁-C₁₀-alkyl;
R¹³⁴ is hydrogen;
R¹³⁸ and R¹³¹⁰ are each phenyl which carries 1 cyano group;
R¹³⁷ and R¹³⁹ are each hydrogen.

In particular, R¹³³ is phenyl which carries 1 cyano group; Likewise even more preferred are compounds of formula (XIII-A), wherein
R¹³³ hydrogen;
R¹³⁴ is phenyl, phenyl which carries 1 cyano group or phenyl which carries 1 substituent selected from C₁-C₁₀-alkyl; in particular phenyl which carries 1 cyano group;
R¹³⁸ and R¹³¹⁰ are each phenyl which carries 1 cyano group;
R¹³⁷ and R¹³⁹ are each hydrogen.

Examples of preferred compounds of formula (XIII-A) are the compounds of formulae (XIII-A.1), (XIII-A.2) (XIII-A.3) and (XIII-A.4)

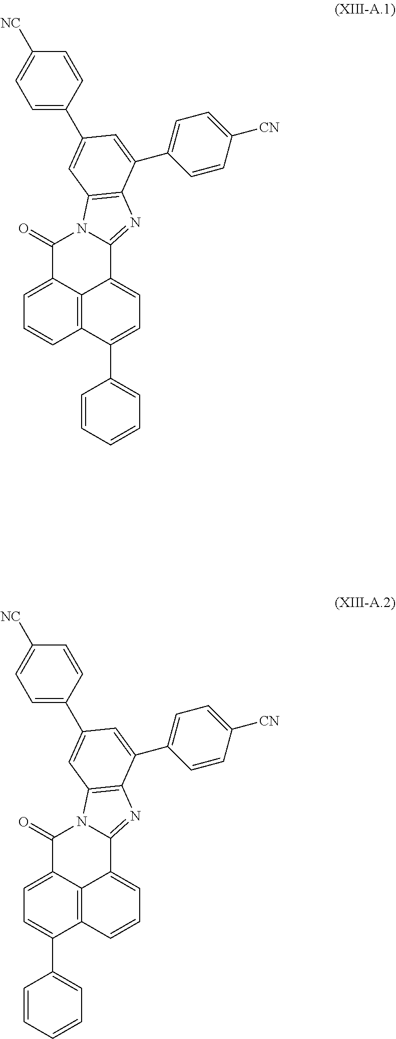 [SMILES: N#Cc1ccc(-c2cc(-c3ccc(C#N)cc3)c3nc4c5ccc(-c6ccccc6)c6cccc(c(=O)n4c3c2)c65)cc1 ; N#Cc1ccc(-c2cc(-c3ccc(C#N)cc3)c3nc4c5cccc6c(-c7ccccc7)ccc(c(=O)n4c3c2)c65)cc1]

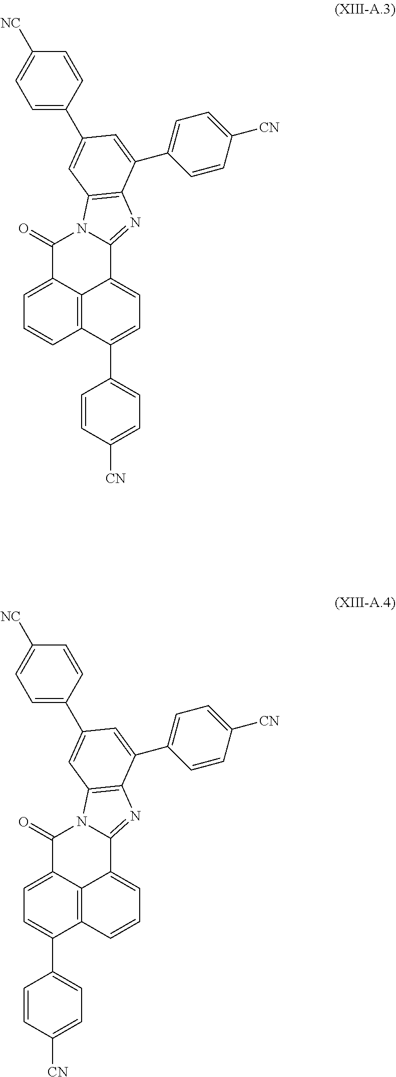 [SMILES: N#Cc1ccc(-c2cc(-c3ccc(C#N)cc3)c3nc4c5ccc(-c6ccc(C#N)cc6)c6cccc(c(=O)n4c3c2)c65)cc1 ; N#Cc1ccc(-c2cc(-c3ccc(C#N)cc3)c3nc4c5cccc6c(-c7ccc(C#N)cc7)ccc(c(=O)n4c3c2)c65)cc1]

### Organic Fluorescent Colorant (B13)

Compounds of formula (XIV) are subject matter of WO 2017/121833. Compounds of formula (XIV) are usually red fluorescent colorants. Preference is given to compounds of formula (XIV), where R¹⁴¹ and R¹⁴² are, independently of each other, selected from phenyl which is unsubstituted or substituted by 1, 2 or 3 C₁-C₆-alkyl; and R¹⁴³, R¹⁴⁴, R¹⁴⁵, R¹⁴⁶, R¹⁴⁷, R¹⁴⁸, R¹⁴⁹, R¹⁴¹⁰, R¹⁴¹¹, R¹⁴¹², R¹⁴¹³, R¹⁴¹⁴, R¹⁴¹⁵, R¹⁴¹⁶, R¹⁴¹⁷ and R¹⁴¹⁸ are each hydrogen. The compound of formula (XIV) as defined above is preferably

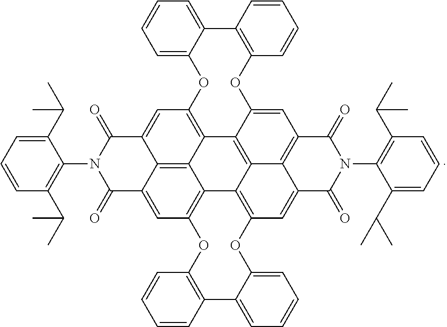 [SMILES: CC(C)c1cccc(C(C)C)c1N1C(=O)c2cc3c4c5c(cc6c7c(cc8c(c9c(cc(c2c49)C1=O)Oc1ccccc1-c1ccccc1O8)c75)C(=O)N(c1c(C(C)C)cccc1C(C)C)C6=O)Oc1ccccc1-c1ccccc1O3]

### Organic Fluorescent Colorant (B14)

Suitable examples of compounds of formula (XV) are for example the perylene derivatives specified in WO 2007/006717, especially at page 1, line 5 to page 22, line 6; in U.S. Pat. No. 4,845,223, especially col. 2, line 54 to col. 6, line 54; in WO 2014/122549, especially at page 3, line 20 to page 9, line 11; in EP 3072887 and WO 2018/065502, especially at page 35, line 34 to page 37, line 29. The compounds of formula (XV) are usually red fluorescent colorants. Preferred are compounds of formula (XV), wherein R¹⁵¹ and R¹⁵² are each independently selected from C₁-C₁₀-alkyl, 2,6-di(C₁-C₁₀-al-kyl) aryl and 2,4-di(C₁-C₁₀-alkyl) aryl. More preferably, R¹⁵¹ and R¹⁵² are identical. Very particularly, R¹³¹ and R¹³² are each 2,6-diisopropylphenyl or 2,4-di-tert-butylphenyl. R¹⁵³ is preferably phenoxy, which is unsubstituted or substituted by 1 or 2 identical or different substituents selected from fluorine, chlorine, C₁-C₁₀-alkyl and phenyl. Preferably, p₁₃ is 2, 3 or 4, in particular 2 or 4.

The compounds of formula (XV) can be prepared in analogy to the methods described for example in WO 2007/006717, U.S. Pat. No. 4,845,223, EP 3072887 and WO 2014/122549.

Suitable organic fluorescent colorants B14 are, for example, N,N′-bis(2,6-diiso-propylphenyl)-1,6,7,12-tetraphenoxyperylene-3,4:9,10-tetracarboximide, N,N′-bis(2,6-diisopropylphenyl)-1,7-di(2,6-diisopropylphenoxy)perylene-3,4:9,10-tetracarboximide, N,N′-bis(2,6-diisopropylphenyl)-1,6-di(2,6-diisopropylphenoxy)perylene-3,4:9,10-tetracarboximide, N,N′-bis(2,6-diisopropylphenyl)-1,7-di(p-tert-octylphe-noxy)perylene-3,4;9,10-tetracarboximide, N,N′-bis(2,6-diisopropylphenyl)-1,7-diphenox-yperylene-3,4;9,10-tetracarboximide, N,N′-bis(2,6-diisopropylphenyl)-1,7-di(2,6-diphe-nylphenoxy)perylene-3,4;9,10-tetracarboximide, N,N′-bis(2,6-diisopropylphenyl)-1,6-di(2,6-diphenylphenoxy)perylene-3,4;9,10-tetracarboximide, N,N′-bis(2,6-diiso-propylphenyl)-1,6,7,12-tetra(2-phenylphenoxy)perylene-3,4:9,10-tetracarboximide, N,N′-bis(2,6-diisopropylphenyl)-1,7-di(2,3-difluorophenoxy)perylene-3,4:9,10-tetracarboximide, N,N′-bis(2,6-diisopropylphenyl)-1,6,7,12-tetra(2,3-difluorophenoxy)perylene-3,4:9,10-tetracarboximide, N,N′-bis(2,6-diisopropylphenyl)-1,6,7,12-tetra(3-fluorophe-noxy)perylene-3,4:9,10-tetracarboximide, N,N′-bis(2,6-diisopropylphenyl)-1,6,7,12-tetra(2,6-difluorophenoxy)perylene-3,4:9,10-tetracarboximide, N,N′-bis(2,6-diiso-propylphenyl)-1,6,7,12-tetra(2,5-difluorophenoxy)perylene-3,4:9,10-tetracarboximide, N,N′-bis(2,6-diisopropylphenyl)-1,6,7,12-tetra(2,3-dichlorophenoxy)perylene-3,4:9,10-tetracarboximide, N,N′-bis(2,6-diisopropylphenyl)-1,6,7,12-tetra(3-chlorophe-noxy)perylene-3,4:9,10-tetracarboximide, N,N′-bis(2,6-diisopropylphenyl)-1,6,7,12-tetra(2,6-dichlorophenoxy)perylene-3,4:9,10-tetracarboximide, N,N′-bis(2,6-diiso-propylphenyl)-1,6,7,12-tetra(2,5-dichlorophenoxy)perylene-3,4:9,10-tetracarboximide. In particular, the organic fluorescent colorant (B14) is selected from compounds (XV-1), (XV-2), (XV-3) and (XV-4)

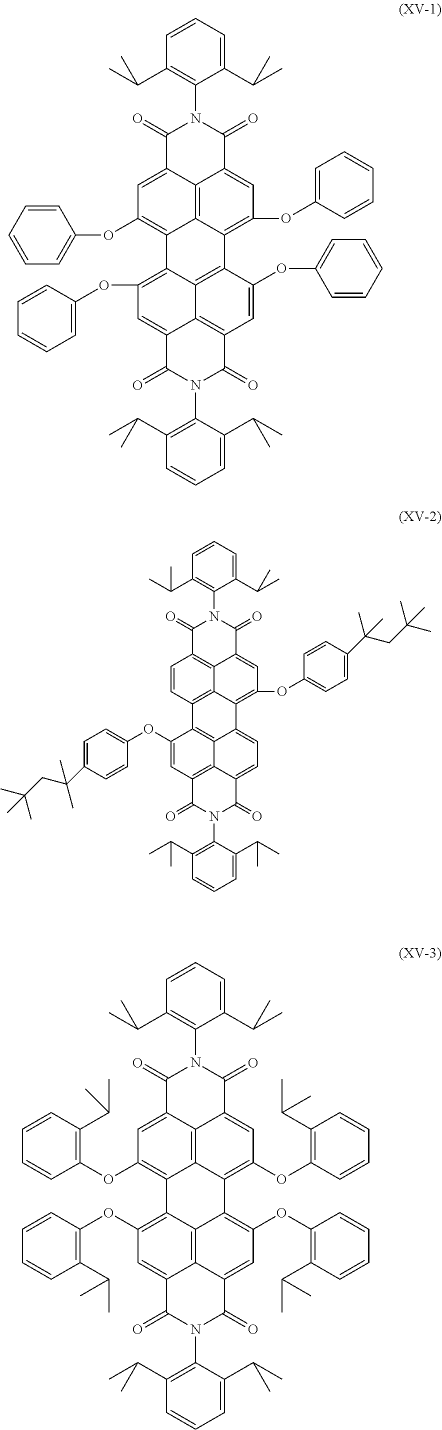 [SMILES: CC(C)c1cccc(C(C)C)c1N1C(=O)c2cc(Oc3ccccc3)c3c4c(Oc5ccccc5)cc5c6c(cc(Oc7ccccc7)c(c7c(Oc8ccccc8)cc(c2c37)C1=O)c64)C(=O)N(c1c(C(C)C)cccc1C(C)C)C5=O ; CC(C)c1cccc(C(C)C)c1N1C(=O)c2ccc3c4c(Oc5ccc(C(C)(C)CC(C)(C)C)cc5)cc5c6c(ccc(c7c(Oc8ccc(C(C)(C)CC(C)(C)C)cc8)cc(c2c37)C1=O)c64)C(=O)N(c1c(C(C)C)cccc1C(C)C)C5=O ; CC(C)c1ccccc1Oc1cc2c3c(cc(Oc4ccccc4C(C)C)c4c5c(Oc6ccccc6C(C)C)cc6c7c(cc(Oc8ccccc8C(C)C)c(c1c34)c75)C(=O)N(c1c(C(C)C)cccc1C(C)C)C6=O)C(=O)N(c1c(C(C)C)cccc1C(C)C)C2=O]

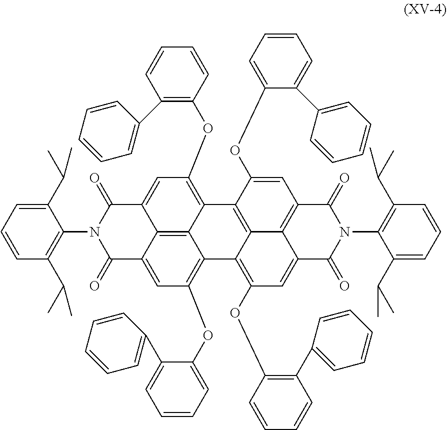 [SMILES: CC(C)c1cccc(C(C)C)c1N1C(=O)c2cc(Oc3ccccc3-c3ccccc3)c3c4c(Oc5ccccc5-c5ccccc5)cc5c6c(cc(Oc7ccccc7-c7ccccc7)c(c7c(Oc8ccccc8-c8ccccc8)cc(c2c37)C1=O)c64)C(=O)N(c1c(C(C)C)cccc1C(C)C)C5=O]

### Organic Fluorescent Colorant (B15)

Compounds of formula (XVI) are subject matter of WO 2019/121602. The compounds of formula (XVI) are usually green fluorescent compounds. Preferred compounds according to the invention are compounds of formula (XVI), wherein the variable X is oxygen.

Preferred compounds are compounds of formula (XVI), wherein R¹⁶², R¹⁶³, R¹⁶⁴ and R¹⁶⁵ are selected from the group consisting of the group consisting of hydrogen and C₆-C₁₀-aryl, which carries one, two or three cyano groups.

More preferably, R¹⁶² and R¹⁶⁴ are selected from the group consisting of C₆-C₁₀-aryl, which carries one, two or three cyano groups. Particularly preferred R¹⁶² and R¹⁶⁴ are each phenyl, which carries one, two or three cyano groups, especially one or two cyano group(s). Particularly preferred R¹⁶³ and R¹⁶⁵ are each hydrogen.

Preferably, the variable A in the compounds of formula (XVI) is a diradical of formula (A.162). In the context of the diradical (A.162), R¹⁶⁶ is preferably selected from the group consisting of the group consisting of hydrogen, linear C₁-C₂₄-alkyl, branched C₃-C₂₄-alkyl, C₆-C₁₀-aryl and C₆-C₁₀-aryl-C₁-C₁₀-alkylene, where the aryl ring in the two last mentioned moieties is unsubstituted or substituted with 1, 2, 3, 4 or 5 identical or different radicals R^{166a}. More preferably, R¹⁶⁶ is selected from the group consisting of linear C₁-C₂₄-alkyl, a radical of formula (B.1), a radical of formula (B.2) and a radical of formula (B.3)

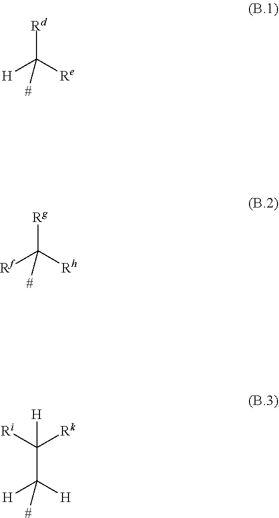

in which
# represents the bonding site to the nitrogen atom;
R^{d} and R^{e}, in the formula (B.1), independently from each other are selected from the group consisting of C₁-C₂₂-alkyl, where the sum of the carbon atoms of the R^{d} and R^{e} radicals is an integer from 2 to 23;
R^{f}, R^{g} and R^{h}, in the formula (B.2) are independently selected from the group consisting of C₁— to C₂₁-alkyl, where the sum of the carbon atoms of the R^{f}, R^{g} and R^{h} radicals is an integer from 3 to 23;
Rⁱ and R^{k}, in the formula (B.3) are independently selected from the group consisting of C₁— to C₂₁-alkyl, where the sum of the carbon atoms of the Rⁱ and R^{k} radicals is an integer from 2 to 22.

In particular, R¹⁶⁶ is linear C₆-C₂₄-alkyl. Herein, specific examples of the radical B.1 are 1-methylethyl, 1-methylpropyl, 1-methylbutyl, 1-methylpentyl, 1-methylhexyl, 1-methylheptyl, 1-methyloctyl, 1-ethylpropyl, 1-ethylbutyl, 1-ethylpentyl, 1-ethylhexyl, 1-ethylheptyl, 1-ethyloctyl, 1-propylbutyl, 1-propylpentyl, 1-propylhexyl, 1 propylheptyl, 1-propyloctyl, 1-butylpentyl, 1-butylhexyl, 1-butylheptyl, 1-butyloctyl, 1 pentylhexyl, 1-pentylheptyl, 1-pentyloctyl, 1-hexylheptyl, 1-hexyloctyl, 1-heptyloctyl. Herein, a specific example of the radical B.2 is tert-butyl.

Herein, specific examples of the radical B.3 are isobutyl, 2-methylbutyl, 2-ethylbutyl, 2-ethylpentyl and 2-ethylhexyl.

Likewise more preferably, R¹⁶⁶ is selected from the group consisting of a radical of formula (C.1), a radical of formula (C.2) and a radical of formula (C.3).

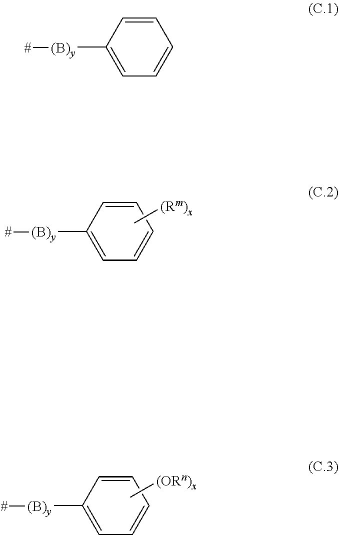

where
# represents the bonding side to the nitrogen atom;
B where present, is a C₁-C₁₀-alkylene group which may be interrupted by one or more nonadjacent groups selected from the group consisting of —O— and —S—;
y is 0 or 1;
each R^{m} in the formula (C.2) is independently of one another selected from the group consisting of C₁-C₂₄-alkyl, C₁-C₂₄-fluoroalkyl, fluorine, chlorine or bromine;
each Rⁿ in the formula (C.3) is independently of one another selected from the group consisting of C₁-C₂₄-alkyl;
x in formulae C.2 and C.3 is 1, 2, 3, 4 or 5.

In the context of R¹⁶⁶, y in formulae C.1, C.2 or C.3 is preferably zero, i.e. B is absent. In the context of R¹⁶⁶, R^{m} in formula C.2 is preferably C₁-C₂₄-alkyl. In the context of R⁶, x in formula C.2 is preferably 1 or 2. In the context of R¹⁶⁶, Rⁿ in formula C.3 is preferably C₁-C₂₄-alkyl. In the context of R⁶, x in formula C.3 is preferably 1 or 2.

According to another embodiment, the variable A in the compounds of formula (XVI) is a diradical of formula (A.163). In the context of (A.163), n in formula (A.163) is preferably zero, one or two. R¹⁶⁷, if present, is preferably selected from the group consisting of cyano, bromine and phenyl which is unsubstituted or carries 1 or 2 radicals selected from the group consisting of C₁-C₄-alkyl.

According to another embodiment, the variable A in the compounds of formula (XVI) is a diradical of formula (A.164). In the context of (A.164), o and p in formula (A.164) are preferably zero, i.e. R¹⁶⁸ and R¹⁶⁹ are both absent. Likewise preferably, in the context of A.4, the sum of o and p is 1, 2, 3 or 4. In this context, R¹⁶⁸ and R¹⁶⁹ are, independently of each other, preferably selected from the group consisting of cyano, bromine, chlorine, C₁-C₄-alkyl, C₁-C₄-alkoxy, C₁-C₄-haloalkyl, phenyl and phenyloxy, wherein phenyl in the two last mentioned radicals is unsubstituted or carries 1, 2 or 3 substituents selected from the group consisting of C₁-C₁₀-alkyl.

Preferred compounds are compounds of formula (XVI), wherein m in formula (XVI) is zero, one or two and when m is one or two, each R¹⁶¹ is independently selected from the group consisting of the group consisting of linear C₁-C₂₄-alkyl, a radical of formula (D.1), a radical of formula (D.2), a radical of formula (D.3), a radical of formula (D.4) and a radical of formula (D.5),

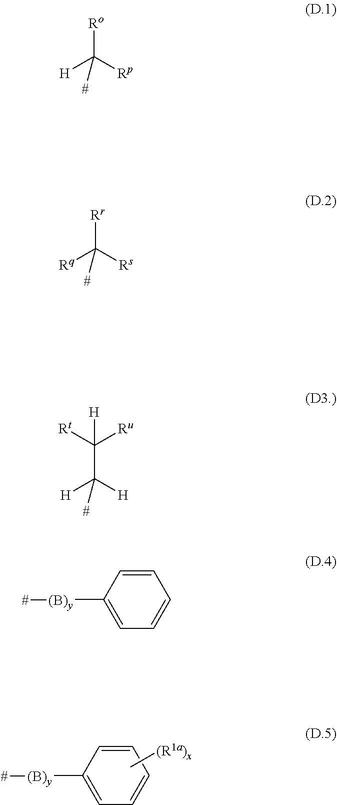

in which
# represents the bonding site to the remainder of the compound of formula I
R^{o} and RP, in the formula (D.1), independently from each other are selected from the group consisting of C₁-C₂₂-alkyl, where the sum of the carbon atoms of the R^{o} and RP radicals is an integer from 2 to 23;
R^{q}, R^{r} and R^{s}, in the formula (D.2) are independently selected from the group consisting of C₁— to C₂₁-alkyl, where the sum of the carbon atoms of the R^{q}, R^{r} and R^{s} radicals is an integer from 3 to 23;
R^{t} and R^{u}, in the formula (D.3) are independently selected from the group consisting of C₁— to C₂₁-alkyl, where the sum of the carbon atoms of the R^{t} and R^{u} radicals is an integer from 2 to 22;
B where present, is a C₁-C₁₀-alkylene group which may be interrupted by one or more nonadjacent groups selected from the group consisting of —O— and —S—;
y in formulae (D.4) and (D.5) is 0 or 1;
x in formula (D.5) is 1, 2 or 3; and
R^{1a} in formula (D.5) is selected from the group consisting of the group consisting of cyano, C₁-C₂₄-alkyl and C₁-C₂₄-alkoxy.

In the context of R¹⁶¹, m in formula (XVI) is preferably zero, i.e. R¹⁶¹ is absent. Likewise more preferably, in the context of R¹⁶¹, m in formula I is one, two or three. R¹⁶¹, if present, is preferably selected from the group consisting of linear C₆-C₂₄-alkyl. Herein, specific examples of R¹⁶¹ are n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosyl, n-uneicosyl, n-docosyl.

In the context of R¹⁶¹, R^{o} and RP in formula (D.1) preferably are each independently C₁-C₁₂-alkyl. In the context of R¹⁶¹, R^{q} and R^{s} in formula (D.2) preferably are each independently C₁-C₆-alkyl and R^{r} in formula (D.2) preferably is branched C₄-C₂₁-alkyl. A specific example of the radical (D.2) is tert-octyl. In the context of R¹⁶¹, x in formula (D.5) preferably is 1 or 2, R^{161a} preferably is cyano or C₁-C₁₂-alkyl. More preferably, R¹⁶¹ is absent or is a radical of formula (D.2) or a radical of formula (D.5), where R^{a} is cyano, y is 0 and x is 1 or 2. Herein, a preferred example of the radical (D.5) is 4-cyanophenyl.

Examples of preferred compounds of formula (XVI) are those depicted below:

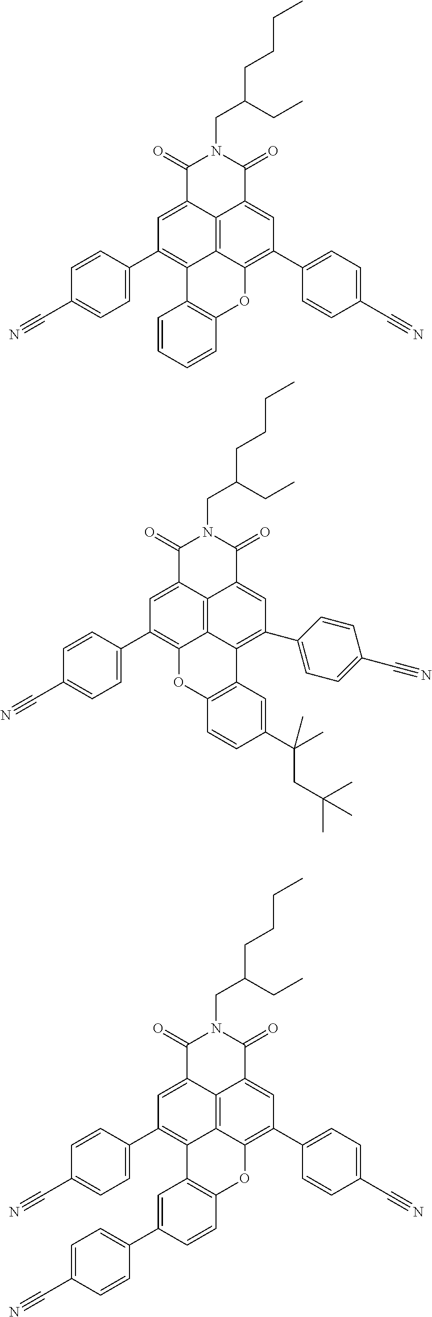 [SMILES: CCCCC(CC)Cn1c(=O)c2cc(-c3ccc(C#N)cc3)c3oc4ccccc4c4c(-c5ccc(C#N)cc5)cc(c1=O)c2c34 ; CCCCC(CC)Cn1c(=O)c2cc(-c3ccc(C#N)cc3)c3oc4ccc(C(C)(C)CC(C)(C)C)cc4c4c(-c5ccc(C#N)cc5)cc(c1=O)c2c34 ; CCCCC(CC)Cn1c(=O)c2cc(-c3ccc(C#N)cc3)c3oc4ccc(-c5ccc(C#N)cc5)cc4c4c(-c5ccc(C#N)cc5)cc(c1=O)c2c34]

Preferably, each compound of groups (B1) to (B15) has a fluorescence quantum yield of at least 80%, measured in a polymer film.

The skilled person will appreciate that the kind and amount of additional color-ant(s) B depend on the intended use of the color converter. When the color converter is intended for use in horticultural lighting, the color converter typically comprises at least one colorant B. Among the colorants (B), those of groups (B6), (B13) and (B14) are preferred.

The concentration of the compound of formula (I) as defined above and, if appropriate, of further colorants in the polymer matrix is set as a function of the thickness of the color converter and the type of polymer. If a thin polymer layer is used, the concentration of the compound of formula (I) and, if appropriate the concentration of further colorants, is generally higher than in the case of a thick polymer layer. Preferably, the concentration of the compound of formula (I) according to the present invention is in the range of from 0.001 to 2% by weight, especially 0.001 to 1% by weight, based on the weight of the polymeric matrix material.

The present invention also relates to a printing ink formulation for security printing. Security printing is the field that deals with the printing of items such as currency, passports, tamper-evident labels, stock certificates, postage stamps, identity cards, etc. The main goal of security printing is to prevent forgery, tampering or counterfeiting.

In the field of automated banknote processing, absorption in the visible wavelength range, IR-absorption and/or fluorescence play an important role. Preference is given to using IR absorbing compounds of formula (I) which are “colourless”, which means that they have a minimal absorption in the VIS range of the electromagnetic spectrum, in particular in the range from 400 to 700 nm. Likewise, preference is given to fluorescent compounds of formula (I). Most of the actually circulating currency carries not only visible coloured printings, but also specific features which are only detect-able in the infrared part of the spectrum and/or by irradiation with UV light. Generally, these features are implemented for use by automatic currency processing equipment, in banking and vending applications (automatic teller machines, automatic vending machines, etc.), in order to recognize a determined currency bill and to verify its authenticity, in particular to discriminate it from replicas made by colour copiers.

All security documents are required to have good stability and durability. In the case of bank notes, these requirements are extreme, as bank notes are subjected to toughest use conditions by the public—they are subjected to material stress by folding, crumpling etc., subjected to abrasion, exposed to weather, exposed to body fluids such as perspiration, laundered, dry-cleaned, ironed etc. —and, after having been subjected to this, are expected to be as legible as when they started. Furthermore, it is essential that the documents nevertheless should have a reasonable life time, ideally of some years, despite suffering the afore-mentioned conditions. During this time, the documents, and thus the inks on them (including invisible security markings), should be resistant to fading or colour change. Hence, any ink used in a security printing process should, when cured, be robust, water-resistant, resistant to various chemicals and flexi-ble. Moreover, as certain states are moving away from the use of paper as the substrate for bank notes, the employed printing ink formulations should be useable on plastics as well as paper. The compound of formula (I), because of its unique application properties, is especially suitable for printing ink formulations that are employed for security printing and in particular for bank notes.

In security printing, the compound of formula (I) is generally added to a printing ink formulation. Suitable printing inks are water-based, oil-based or solvent-based printing inks, based on pigment or dye, for inkjet printing, flexographic printing, screen printing, intaglio printing, offset printing, laser printing or letterpress printing and for use in electrophotography. Printing inks for these printing processes usually comprise solvents, binders, and also various additives, such as plasticizers, antistatic agents or waxes. Printing inks for offset printing and letterpress printing are usually formulated as high-viscosity paste printing inks, whereas printing inks for flexographic printing and intaglio printing are usually formulated as liquid printing inks with comparatively low viscosity.

In the context of the present invention, the expression “printing ink” also encompasses formulations that in addition to the compound of formula (I) comprise a colorant. The expression “printing ink” also encompasses printing lacquers that comprise no colorant.

The printing ink formulation for security printing according to the invention preferably comprises
a) at least one compound of the formula (I) according to the invention as described above;
b) a polymeric binder;
c) optionally a solvent;
d) optionally at least one colorant different from the compound of formula (I); e) optionally at least one further additive; and
f) optionally at least one photoinitiator.

In a preferred embodiment, the printing ink formulation comprises
a) 0.0001 to 25% by weight of at least one compound of the formula (I) as defined above;
b) 5 to 99.9999% by weight of at least one polymeric binder;
c) 0 to 94.9999% by weight of at least one solvent;
d) 0 to 25% by weight of at least one colorant different from the compound of formula (I);
e) 0 to 25% by weight of at least one further additive; and
f) 0 to 14% by weight at least one photoinitiator;
wherein the sum of components a) to f) adds up to 100%.

Suitable components of printing inks are conventional and are well known to those skilled in the art. Examples of such components are described in “Printing Ink Manual”, fourth edition, Leach R. H. et al. (eds.), Van Nostrand Reinhold, Wokingham, (1988). Details of printing inks and their formulation are also disclosed in “Printing Inks”-Ullmann's Encyclopedia of Industrial Chemistry, Sixth Edition, 1999 Electronic Release. A formulation of an IR-absorbing intaglio ink formulation is described in US20080241492A1. The disclosure of the afore-mentioned documents is incorporated herein by reference.

The printing ink formulation according to the invention contains in general from 0.0001 to 25% by weight, preferably from 0.001 to 5% by weight, in particular from 0.01 to 0.1% by weight, based on the total weight of the printing ink formulation, of at least one compound of formula (I), component a). The good solubility/dispersibility of the compound of formula (1) in the printing ink formulation allows for incorporating high amounts of absorber into the formulation. Thus, it is possible to obtain formulations containing high amounts of the visible light/IR absorbing compound of formula (1) and comparatively low amount of other ingredients which might affect the absorbance of the compound of formula (1) or other properties.

The printing ink formulation according to the invention contains in general from 5 to to 99.9999% by weight, often from 5 to 75% by weight, preferably from 10 to 60% by weight, more preferably from 15 to 40% by weight, based on the total weight of the printing ink formulation, of a binder [component b)].

Suitable polymeric binders b) for the printing ink formulation according to the invention are for example selected from natural resins, phenol resins, phenol-modified resins, alkyd resins, polystyrene homo- and copolymers, terpene resins, silicone resins, polyurethane resins, urea-formaldehyde resins, melamine resins, polyamide resins, polyacrylates, polymethacrylates, chlorinated rubber, vinyl ester resins, acrylic resins, epoxy resins, nitrocellulose, hydrocarbon resins, cellulose acetate, and mixtures thereof.

The printing ink formulation according to the invention can also comprise components that form a polymeric binder by a curing process. Thus, the printing ink formulation according to the invention can also be formulated to be energy-curable, e.g. able to be cured by UV light or EB (electron beam) radiation. In this embodiment, the binder comprises one or more curable monomers and/oligomers. Corresponding formulations are known in the art and can be found in standard textbooks such as the series “Chemistry & Technology of UV & EB Formulation for Coatings, Inks & Paints”, published in 7 volumes in 1997-1998 by John Wiley & Sons in association with SITA Technology Limited.

Suitable monomers and oligomers (also referred to as prepolymers) include epoxy acrylates, acrylated oils, urethane acrylates, polyester acrylates, silicone acrylates, acrylated amines, and acrylic saturated resins. Further details and examples are given in “Chemistry & Technology of UV & EB Formulation for Coatings, Inks & Paints”, Volume 1l: Prepolymers & Reactive Diluents, edited by G Webster.

If a curable polymeric binder is employed, it may contain reactive diluents, i.e. monomers which act as a solvent and which upon curing are incorporated into the polymeric binder. Reactive monomers are typically chosen from acrylates or methacrylates, and can be monofunctional or multifunctional. Examples of multifunctional monomers include polyester acrylates or methacrylates, polyol acrylates or methacrylates, and polyether acrylates or methacrylates.

The printing ink formulation according to the invention contains in general from 0 to 94.9999% by weight, preferably from 5 to 90% by weight, in particular from 10 to 85% by weight, based on the total weight of the printing ink formulation, of a solvent c).

Suitable solvents c) are selected from water, organic solvents and mixtures thereof. For the purpose of the invention, reactive monomers which also act as solvent are regarded as part of the afore-mentioned binder component b).

Examples of solvents c) comprise water; alcohols, e.g. ethanol, 1-propanol, 2-propanol, ethylene glycol, propylene glycol, diethylene glycol and ethoxy propanol; esters, e.g. ethyl acetate, isopropyl acetate, n-propyl acetate and n-butyl acetate; hydrocarbons, e.g. toluene, xylene, mineral oils and vegetable oils, and mixtures thereof.

The printing ink formulation according to the invention may contain one or more additional colorants d) which impart a specific optical property (i.e. colorants or components which are different from the compound of formula (1)). Preferably, the printing ink formulation contains in from 0 to 25% by weight, more preferably from 0.1 to 20% by weight, in particular from 1 to 15% by weight, based on the total weight of the printing ink formulation, of a colorant d).

Suitable colorants d) are selected conventional dyes, and in particular conventional pigments. The term “pigment” is used in the context of this invention comprehen-sively to identify all pigments and fillers, examples being colour pigments, white pigments, and inorganic fillers. These include inorganic white pigments such as titanium dioxide, preferably in the rutile form, barium sulfate, zinc oxide, zinc sulfide, basic lead carbonate, antimony trioxide, lithopones (zinc sulfide+barium sulfate), or colored pigments, examples being iron oxides, carbon black, graphite, zinc yellow, zinc green, ul-tramarine, manganese black, antimony black, manganese violet, Paris blue or Schweinfurt green. Besides the inorganic pigments the printing ink formulation of the invention may also comprise organic colour pigments, examples being sepia, gam-boge, Cassel brown, toluidine red, para red, Hansa yellow, indigo, azo dyes, anthraqui-nonoid and indigoid dyes, and also azo, dioxazine, quinacridone, perylene, quinoph-thalone, diketopyrrolopyrrole, phthalocyanine, isoindoline, isoindolinone, and metal complex pigments. Also suitable are synthetic white pigments with air inclusions to in-crease the light scattering, such as the Rhopaque® dispersions. Suitable fillers are, for example, aluminosilicates, such as feldspars, silicates, such as kaolin, talc, mica, mag-nesite, alkaline earth metal carbonates, such as calcium carbonate, in the form for example of calcite or chalk, magnesium carbonate, dolomite, alkaline earth metal sul-fates, such as calcium sulfate, silicon dioxide, etc.

Although they are invisible to the human eye, in terms of the present invention, further IR absorbers are also considered to belong to colorants d). Preferred further IR absorbers are selected from the group consisting of polymethines, phthalocyanines, quinone-diimmonium salts, aminium salts, inorganic IR absorbers and mixtures thereof.

The printing ink formulation may comprise at least one colorant d) for establishing a desired hue, especially transparent organic pigments and dyes, for example C.I. Pigment Yellow 13, 14, 17, 74, 138, 139, 147, 150, 151, 155, 183, 185 192 and 196, C.I. Pigment Orange 34, 64, 70, C.I. Pigment Red 48:1, 48:2, 57:1, 149, 178 and 179, 181, 263, C.I. Pigment Violet 19, 37 and 29, C.I. Pigment Blue 15, 15:1, 15:3 and 15:4, 15:6, 16, 60, C.I. Pigment Green 7 and 36, C.I. Solvent Yellow 14, 21, 93, 130, 133, 145, 162, 163, C.I. Solvent Red 52, 135, 195, 213, 214 and 225, C.I. Solvent Blue 35, 45, 67, 68, 97, 104, 122, 132, C.I. Solvent Violet 13, 46, 49, C.I. Solvent Green 3, 5 and 28, C.I. Solvent Orange 47, 60, 86, 114, and 163, C.I. Solvent Brown 35, 53, and also C.I. Disperse Yellow 54, 87, 201, C.I. Disperse Orange 30, C.I. Disperse Red 60 and C.I. Disperse Violet 57.

The printing ink formulation according to the invention may contain at least one additive e). Preferably, the printing ink formulation contains from 0 to 25% by weight, more preferably from 0.1 to 20% by weight, in particular from 1 to 15% by weight, based on the total weight of the printing ink formulation, of at least one component e).

Suitable additives (component e) are selected from plasticizers, waxes, siccatives, antistatic agents, chelators, antioxidants, stabilizers, adhesion promoters, surfac-tants, flow control agents, defoamers, biocides, thickeners, etc. and combinations thereof. These additives serve in particular for fine adjustment of the application-related properties of the printing ink, examples being adhesion, abrasion resistance, drying rate, or slip.

A further possible additive group is that of additives which likewise modify the vis-ual appearance, the mechanical properties or else the tactile properties, for example matting agents, such as titanium dioxide, chalk, barium sulfate, zinc sulfide, fillers, such as nanoparticulate silicon dioxide, aluminium hydroxide, clay and other sheet silicates, glass fibers and glass spheres.

In the case of printing ink formulations to be cured by UV radiation, it is usually necessary to include at least one photoinitiator (component f) to initiate the curing reaction of the monomers upon exposure to UV radiation. Photoinitiators are understood to be substances which, when exposed to UV radiation, i.e. light with a wavelength below 420 nm, in particular below 410 nm, decompose with the formation of radicals and thus trigger a polymerization of the ethylenically unsaturated double bonds. Therefore, such photoinitiators are tuned to the wavelength of the light source and typically have at least one absorption band which has a maximum in the range from 220 to 420 nm, in particular in the range from 230 to 410 nm that.

Examples of useful photoinitiators f) can be found in standard textbooks such as “Chemistry & Technology of UV & EB Formulation for Coatings, Inks & Paints”, Volume III, “Photoinitiators for Free Radical Cationic and Anionic Polymerisation”, 2nd edition, by J. V. Crivello & K. Dietliker, edited by G. Bradley and published in 1998 by John Wiley & Sons in association with SITA Technology Limited. It may also be advantageous to include a sensitizer in conjunction with the photoinitiator in order to achieve efficient curing. Preferred photoinitiators are especially alpha-hydroxyalkylphenones, alpha-dialkoxyacetophenones, phenylglyoxalic acid esters, benzophenones, benzoins and acylphosphinoxides.

In another preferred embodiment, the at least one compound of formula (1) is incorporated in a UV curable printing formulation. In this case, the formulation typically comprises
a) 0.0001 to 25% by weight of at least one compound of the formula (1) as defined above;
b) 5 to 97.9999%, preferably 5 to 70% by weight of at least one UV curable polymeric binder;
c) 1 to 55%, preferably 10 to 49.9999% by weight of at least one reactive diluent;
d) 0 to 25% by weight of a colorant different from the compound of the formula (1);
e) 0 to 25% by weight of at least one further additive; and
f) 1 to 14% of a photoinitiator;
wherein the sum of components a) to f) adds up to 100%.

According to a further embodiment, the polymeric binder b) of the printing ink formulation is an oxidative drying resin.

Oxidation is the classical drying mechanism for lithographic inks, involving the ox-ygen-induced free radical polymerisation of unsaturated (drying) polymers, such as vegetable oils, for example, linseed oil and tung oil. The oxidant is atmospheric oxygen. It is a chemical process which can be catalysed (accelerated) by small amounts of appropriate metal, usually transition metal, driers, such as cobalt and/or manganese catalysts (siccatives). Examples of thermally or oxidatively drying binders are alkyd resins, such as in particular long oil alkyd resins, polyamide resins, (meth)acrylic resins, polyurethane resins, phenolic resins, vinyl resins, rosin modified maleic resins and varnishes made by cooking a resin, such as an alkyd, polyurethane or phenolic resin, with an oxidative drying oil, such as tung oil, linseed oil, poppy seed oil or perilla oil, as well as mixtures of these resins and varnishes. The aforementioned resins and varnishes are well known and e.g. described in more detail by R. van Gorkum et al., Coordination Chemistry Reviews 249 (2005) 1709-1728; J. F. Black, J. Am. Chem. Soc., 1978, 100, 527, J. Mallégol et al., Prog. Org. Coatings 39 (2000) 107-113, The Printing ink manual, R. H. Leach and R. J. Pierce, Springer Edition, 5^{th} Edition and commercially available, e.g. from Epple Druckfarben AG.

The printing ink formulations according to the invention are advantageously prepared in a conventional manner, for example by mixing the individual components. As mentioned above, at least one compound of formula (1) according to the invention is present in the printing ink formulation. Additional colorants may be employed in the printing ink formulation of the invention or in a separate ink formulation. When additional colorants are to be employed in a separate formulation, the order of application of the printing ink formulation according to the invention is usually not relevant. The printing ink formulation according to the invention can for example be applied first and then be overprinted with conventional printing inks. But it is also possible to reverse this sequence or, alternatively, to apply the printing ink formulation according to the invention in a mixture with conventional printing inks. In every case the prints are readable with suitable light sources.

Primers can be applied to the substrate prior to the printing ink formulation according to the invention. By way of example, the primers are applied in order to improve adhesion to the substrate. It is also possible to apply additional printing lacquers, e.g. in the form of a covering to protect the printed image. Additional printing lacquers may also be applied to serve aesthetic purposes, or serve to control application-related properties. By way of example, suitably formulated additional printing lacquers can be used to influence the roughness of the surface of the substrate, the electrical properties, or the water-vapour-condensation properties. Printing lacquers are usually applied in-line by means of a lacquering system on the printing machine employed for printing the printing ink formulation according to the invention.

The printing ink formulations according to the invention are also suitable for use in multilayer materials. Multilayer materials are e.g. composed of two or more plastics foils, such as polyolefin foils, metal foils, or metallised plastics foils, which are bonded to one another, by way of example, via lamination or with the aid of suitable laminating adhesives. These composites may also comprise other functional layers, such as odour-barrier layers or water-vapour barriers.

These layers may additionally comprise one or more UV absorbers. UV absorbers are well known in the plastics, coatings and cosmetic industry.

The present invention also relates to a method for producing a security feature or a security document, which comprises applying a printing ink formulation to a substrate by a printing process.

In a further aspect the invention provides a security document, the security document comprising a substrate, a cured ink which ink comprises at least one compound of the formula (I) as defined above.

Yet in a further aspect, the invention provides a security document, obtainable by a printing process, wherein a printing ink formulation is used that comprises at least one compound of formula (I) as defined above. “Obtainable” indicates that the security document can also be obtained by a process different from printing, e.g. by manual application, or by spin coating or slot die coating. This notwithstanding, the resulting security document comprises at least one compound of formula (I) as defined above. Preferably however, the security document is indeed obtained by a printing process, in which process the printing ink formulation of the invention is used that comprises at least one compound of formula (1) as defined above.

The printing ink formulations of the invention may be applied by a printing process preferably selected from the group consisting of offset-printing processes, rotogra-vure printing processes, silkscreen printing processes, copperplate intaglio printing processes, flexographic printing processes, letterpress printing processes; more preferably by offset-printing processes and copperplate intaglio printing processes. The aforementioned printing techniques are well known to a skilled person.

In this context, the term “security feature” is in particular a specific image that is printed on a substrate; and term “substrate” means any object which is intended to be furnished with a security feature, or intended to be converted into a security document by applying the ink formulation in order to produce a security feature.

The term “security document” means any document intended to be protected against forgery or counterfeiting. Such security documents include in particular value documents and value commercial goods. Typical example of value documents include e.g. banknotes, documents, deeds, tickets, checks, vouchers, fiscal stamps and tax labels, agreements and the like, identity documents such as passports, identity cards, vi-sas, driving licenses, bank cards, credit cards, transactions cards, access documents or cards, entrance tickets, public transportation tickets or titles and the like, preferably banknotes, identity documents, right-conferring documents, driving licenses and credit cards. The term “value commercial good” refers e.g. to packaging materials, in particular for cosmetic articles, nutraceutical articles, pharmaceutical articles, alcohols, tobacco articles, beverages or foodstuffs, electrical/electronic articles, fabrics or jewelry, i.e. articles that shall be protected against counterfeiting and/or illegal reproduction in order to warrant the content of the packaging like for instance genuine drugs. Examples of these packaging materials include e.g. labels, such as authentication brand labels, tamper evidence labels and seals. It is pointed out that the disclosed substrates, value documents and value commercial goods are given exclusively for exemplifying purposes, without restricting the scope of the invention.

The present invention also relates to a method of detecting the authenticity of the security document as defined above, comprising the steps of:
a) measuring an absorbance, reflectance or transmittance spectrum of the security document in the visible/near infra-red range of the electromagnetic spectrum; and
b) comparing the spectrum measured under a) and/or information derived therefrom with a corresponding spectrum and/or information of an authentic security element.
   Since the inventive printing formulation comprises the compound of formula (1), these compounds are consequently also part of the image coated or printed on the substrate. By using devices capable of measuring visible/NIR radiation it is then possible to detect the otherwise undetectable image on the basis of the extent to which the visible/NIR radiation is absorbed, in particular the radiated wavelengths in the range of 700 to 1100 nm, especially in the range of 700 to 900 nm. This way the specific security image can be identified on the substrate.

The printing formulations of the invention are particularly suitable for this type of security printing, as they enable security images exhibiting remission (radiation reflectance and scattering) of wavelengths in the range of 700 to 1100 nm, especially of 700 to 900 nm, that is reduced by at least 40%, in particular at least 50%, in comparison to the blank substrate.

The invention will be illustrated in detail by the examples.

### PREPARATION EXAMPLES

### I. Preparation of Intermediate Compounds

The starting materials used in the examples are either commercially available or can be synthesized by the average skilled person trained in organic chemistry following routine laboratory practice as outlined, for example, in the examples below.

### a1) 2,4-Bis(1,1,3,3-tetramethylbutyl)phenol

A mixture of 10.16 g (48 mmol) of 4-tert.-octylphenol, 5.41 g (48 mmol) of 2,4,4-trimethylpent-1-ene and 100 mL of dichloromethane was cooled to −20° C. 5 mL of methanesulfonic acid were slowly added and the mixture was kept between −18° C. and −12° C. for three hours. Water was added, phases separated and the organic phase repeatedly washed with water, dried with MgSO₄ and evaporated. The product was purified by column chromatography on silica using cyclohexane and toluene. 10.4 g (68%) of a colorless oil were isolated.
R^{f}=(toluene:ethyl acetate=10:1)=0.83.

### a2) 2,6-Di-isopropyl-4-tert.-octylphenol

A mixture of 10.00 g (54.4 mmol) of 2,6-diisopropylphenol, 6.18 g (54.4 mmol) of 2,4,4-trimethylpent-1-ene and 100 mL of dichloromethane was cooled to −12° C. Then 7.44 g (77 mmol) of methane sulfonic acid were slowly added within 15 minutes. The reaction mixture was stirred at −18° C. for 2 hours. Then 100 mL of water were added, the phases were separated and dried with MgSO₄, organics were evaporated and the residue was subjected to column chromatography using silica gel and cyclohexane toluene 90:10 for purification. 15.07 g (81%) of a yellow oil were obtained.

### a3) N,N′-Bis(2,6-diisopropylphenyl)-1,6,9,14-tetrabromoterrylen-3,4,11,12-tetracarboxylic acid diimide

The title compound was prepared according to EP 1802 622.

### a4) N,N′-Bis(2,6-diisopropylphenyl)-1,6, 8, 11,16,18-hexabromoquaterrylen-3,4;13,14-tetracarboxylic acid diimide

The title compound was prepared according to WO 96/22332.

### II. Preparation of Compounds of the Formula (1)

### Example 1

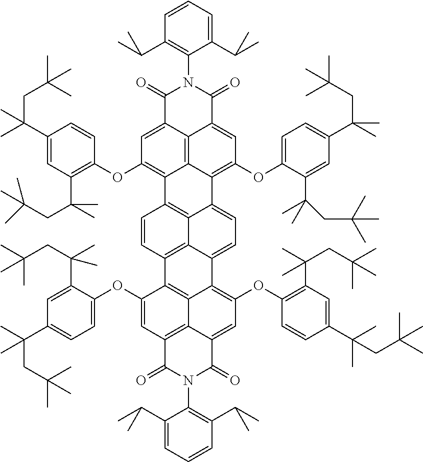 [SMILES: CC(C)c1cccc(C(C)C)c1N1C(=O)c2cc(Oc3ccc(C(C)(C)CC(C)(C)C)cc3C(C)(C)CC(C)(C)C)c3c4ccc5c6c(Oc7ccc(C(C)(C)CC(C)(C)C)cc7C(C)(C)CC(C)(C)C)cc7c8c(cc(Oc9ccc(C(C)(C)CC(C)(C)C)cc9C(C)(C)CC(C)(C)C)c(c9ccc(c%10c(Oc%11ccc(C(C)(C)CC(C)(C)C)cc%11C(C)(C)CC(C)(C)C)cc(c2c3%10)C1=O)c4c59)c86)C(=O)N(c1c(C(C)C)cccc1C(C)C)C7=O]

A mixture of 2.80 g (2.4 mmol) of N,N′-bis(2,6-diisopropylphenyl)-1,6,9,14-tetrabro-moterrylen-3,4;11,12-tetracarboxylic acid diimide from a3), 4.89 g (14.6 mmol) of the phenol from a1), 750 mg (5.3 mmole) of K₂CO₃ and 50 mL of N-methylpyrrolidone were heated to 90° C. for two days. Afterwards the temperature was raised to 130° C. for 2 hours. Further 340 mg of potassium carbonate and 376 mg of the phenol from a1) were added and the reaction mixture kept at this temperature for 20 hours. The mixture was cooled to room temperature, 50 mL of 20% hydrochloric acid were added and the precipitate was filtered, washed with water and dried under reduced pressure. The residue was subjected to column chromatography using cyclohexane/toluene as eluent to obtain 940 mg of the green title product.
R^{f} (petroleum ether toluene 30:70)=0.5; lambda max absorption (CH₂Cl₂): 694 nm

### Example 2

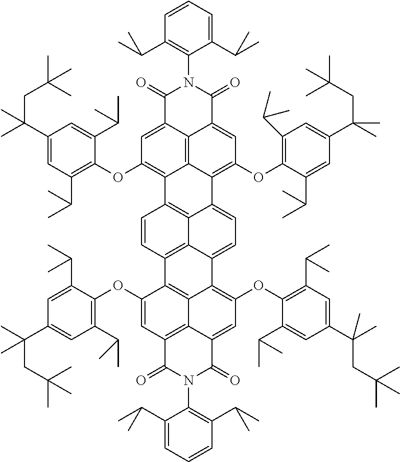 [SMILES: CC(C)c1cc(C(C)(C)CC(C)(C)C)cc(C(C)C)c1Oc1cc2c3c(cc(Oc4c(C(C)C)cc(C(C)(C)CC(C)(C)C)cc4C(C)C)c4c5ccc6c7c(Oc8c(C(C)C)cc(C(C)(C)CC(C)(C)C)cc8C(C)C)cc8c9c(cc(Oc%10c(C(C)C)cc(C(C)(C)CC(C)(C)C)cc%10C(C)C)c(c%10ccc(c1c34)c5c%106)c97)C(=O)N(c1c(C(C)C)cccc1C(C)C)C8=O)C(=O)N(c1c(C(C)C)cccc1C(C)C)C2=O]

A mixture of 6 mL of N-methylpyrrolidone, 502 mg (392 mmol) of N,N′-bis(2,6-diiso-propylphenyl)-1,6,9,14-tetrabromoterrylen-3,4,11,12-tetracarboxylic acid diimide from a3), 537 mg (1.72 mmol) of the phenol from a2) and 190 mg (1.17 mmol) of potassium carbonate was heated to 105° C. for 6 hours. The mixture was cooled to room temperature, and 200 mL of water and 100 mL of tert-butyl methylether were added. The phases were separated and the organic phase was washed three times with water, dried with Na₂SO₄ evaporated and the residue was subjected to column chromatography using cyclohexane toluene mixtures to yield 260 mg (34%) of the blue title product.
R^{f} (toluene)=0.76; lambda max absorption (CH₂Cl₂): 699 nm

### Example 3

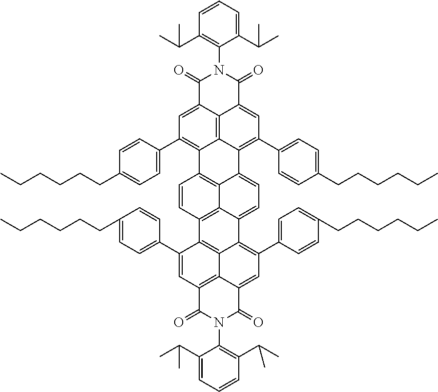 [SMILES: CCCCCCc1ccc(-c2cc3c4c(cc(-c5ccc(CCCCCC)cc5)c5c6ccc7c8c(-c9ccc(CCCCCC)cc9)cc9c%10c(cc(-c%11ccc(CCCCCC)cc%11)c(c%11ccc(c2c45)c6c%117)c%108)C(=O)N(c2c(C(C)C)cccc2C(C)C)C9=O)C(=O)N(c2c(C(C)C)cccc2C(C)C)C3=O)cc1]

A mixture of 200 mg (0.157 mmol) of N,N′-bis(2,6-diisopropylphenyl)-1,6,9,14-tetrabro-moterrylen-3,4;11,12-tetracarboxylic acid diimide from a3), 142.3 mg (0.691 mmol) of 4-n-hexylphenylboronic acid, 65.1 mg (0.471 mmole) of K₂CO₃, 0.2 mg (0.157 μm) of tetrakistriphenylphopshinepalladium and 5 mL of toluene were heated to 85° C. for a day. The mixture was cooled to room temperature, water and methylene chloride were added and the phases were separated. To the organic phase, brine was added, the phases separated, dried with MgSO₄, the precipitate was filtered, and the residue evaporated. The crude product was purified by column chromatography on silica using cyclohexane and toluene to yield 106.7 mg (46%) of the turquoise title product. R^{f} (toluene)=0.31; lambda max absorption (CH₂Cl₂): 720 nm

### Example 4

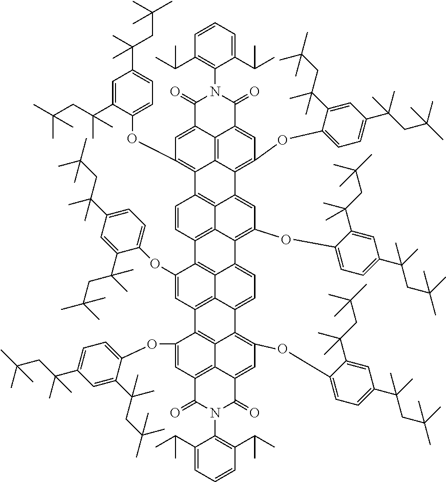 [SMILES: CC(C)c1cccc(C(C)C)c1N1C(=O)c2cc(Oc3ccc(C(C)(C)CC(C)(C)C)cc3C(C)(C)CC(C)(C)C)c3c4ccc5c6c(Oc7ccc(C(C)(C)CC(C)(C)C)cc7C(C)(C)CC(C)(C)C)cc7c8c(Oc9ccc(C(C)(C)CC(C)(C)C)cc9C(C)(C)CC(C)(C)C)cc9c%10c(cc(Oc%11ccc(C(C)(C)CC(C)(C)C)cc%11C(C)(C)CC(C)(C)C)c(c%11ccc(c%12c(Oc%13ccc(C(C)(C)CC(C)(C)C)cc%13C(C)(C)CC(C)(C)C)cc(c%13c(Oc%14ccc(C(C)(C)CC(C)(C)C)cc%14C(C)(C)CC(C)(C)C)cc(c2c3%13)C1=O)c4c5%12)c6c%117)c%108)C(=O)N(c1c(C(C)C)cccc1C(C)C)C9=O]

A flask was heated and cooled down under argon. Then a mixture of 500 mg (0.3 mmol -1 eq) of N,N′-bis(2,6-diisopropylphenyl)-1,6,8,11,16,18-hexabromoquaterrylen-3,4,13,14-tetracarboxylic acid diimide from a4), 650 mg (1.9 mmol-6.6 eq) of the phenol from a1), 135 mg (1.0 mmol-3.3 eq) K₂CO₃ and 10 mL of anhydrous N-methylpyrrolidone were heated to 97° C. for 20 h. The mixture was cooled to room temperature, water and methylene chloride were added and the phases were separated. To the organic phase NaCl solution was added, phases separated, dried with MgSO₄ and evaporated. The residue was subjected to column chromatography using cyclohexane/toluene as eluent to yield 200 mg of a green product.
R^{f} (toluene)=0.81; lambda max absorption (CH₂Cl₂): 777 nm

### Example 5

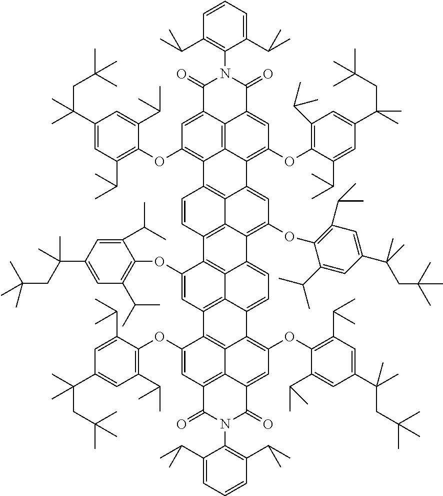 [SMILES: CC(C)c1cc(C(C)(C)CC(C)(C)C)cc(C(C)C)c1Oc1cc2c3c(cc(Oc4c(C(C)C)cc(C(C)(C)CC(C)(C)C)cc4C(C)C)c4c5cc(Oc6c(C(C)C)cc(C(C)(C)CC(C)(C)C)cc6C(C)C)c6c7ccc8c9c(Oc%10c(C(C)C)cc(C(C)(C)CC(C)(C)C)cc%10C(C)C)cc%10c%11c(cc(Oc%12c(C(C)C)cc(C(C)(C)CC(C)(C)C)cc%12C(C)C)c(c%12cc(Oc%13c(C(C)C)cc(C(C)(C)CC(C)(C)C)cc%13C(C)C)c(c%13ccc(c1c34)c5c%136)c7c8%12)c%119)C(=O)N(c1c(C(C)C)cccc1C(C)C)C%10=O)C(=O)N(c1c(C(C)C)cccc1C(C)C)C2=O]

A mixture of 1.00 g (0.643 mmol) of N,N′-bis(2,6-diisopropylphenyl)-1,6,8, 11,16,18-hexabromoquaterrylen-3,413,14-tetracarboxylic acid diimide from a4), 1.45 g (4.243 mmol) of the phenol from a2), 296 mg (2.12 mmol) of potassium carbonate and 20 mL of N-methylpyrrolidone was heated 95° C. for 20 hours. The workup and purification were done as described in example 4 to yield 140 mg (8.1%) of a green oil. R^{f} (toluene)=0.75; lambda max absorption (CH₂Cl₂): 786 nm

### III. Preparation of Comparative Examples

### Comparative Example 1

### N,N′-bis(2,6-diisopropylphenyl)-1,6,9,14-tetrakis(4-tert-octylphenoxy)terrylene-3,4,11,12-tetracarboximide

The title compound was prepared as described in example 29 of WO 2003/104232; lambda max absorption (CH₂Cl₂): 677.09 nm

### Comparative Example 2

### N,N′-Bis(2,6-diisopropylphenyl)-1,6,9,14-tetra(2,6-diisopropylphenoxy)terrylene-3,4,11,12-tetracarboximide

The title compound was prepared as decribed in example 2 of WO 2007/006717.

### IV. Photophysical Results

### Emission Spectra

Here and in the following, the abbreviations have the following meanings:
QY: fluorescence quantum yield
Abs. int.: absorption in integrating sphere
Fluo.: product of absorption and QY measured in integrating sphere

### Measurement of Quantum Yields and Absolute Absorption in a Polycarbonate Film

Fluorescence quantum yields (QY) of the analysis samples were measured with the C₉₉₂₀-02 quantum yield measuring system from Hamamatsu. This was done by illuminating each of the samples in an integrating sphere (Ulbricht sphere). By comparison with the reference measurement in the Ulbricht sphere without sample, the unabsorbed fraction of the excitation light and the fluorescent light emitted by the sample are determined by means of a CCD spectrometer. Integration of the intensities of the spectrum of the unabsorbed excitation light and of that of the emitted fluorescent light gives the degree of absorption and fluorescence intensity, respectively, and thus the fluorescence quantum yield of each sample can be calculated.

### Preparation of the Sample

For preparation of the samples, the dye in question, polycarbonate (PC, Makrolon 2808) and TiO₂ (Kronos 2233, 0.5% by weight) were mixed together according to the desired concentration (see Table I-V). The concentrations are given relative to the amount of polycarbonate used. Then methylene chloride was added and the mixtures were stirred overnight. The solutions/dispersions were coated onto a glass surface by doctor blading. After the solvent had dried off for 2 hours, the film was detached from the glass and dried in vacuum at 50° C. Samples were cut from the foils and had a thickness of 67 μm.

TABLE I
Results for the compound of example
1 in free-standing polycarbonate film

| | Excitation | | | Abs | |
|---|---|---|---|---|---|
| | wavelength/nm | QY | lambdaₘₐₓ/nm | int. | Fluo. |
| 0.003% PC | 635 | 69% | 711 | 13% | 9% |
| 0.003% PC | 685 | 69% | 708 | 18% | 13% |
| 0.005% PC | 600 | 62% | 714 | 11% | 7% |
| 0.005% PC | 635 | 69% | 714 | 21% | 14% |
| 0.005% PC | 660 | 68% | 715 | 17% | 12% |
| 0.07% in PC | 600 | 57% | 777 | 49% | 28% |
| 0.1% in PC | 600 | 55% | 778 | 59% | 33% |
| 0.2% in PC | 600 | 50% | 783 | 74% | 37% |
| 0.5% in PC | 600 | 41% | 787 | 90% | 37% |

As can be seen, a bathochrome shift of the emission occurs with increasing concentration of the fluorescent dye.

TABLE II
Results for the compound of example
2 in free-standing polycarbonate film

| | Excitation | | | Abs | |
|---|---|---|---|---|---|
| | wavelength/nm | QY | lambdaₘₐₓ/nm | int. | Fluo. |
| 0.028% PC* | 640 | 59% | 785 | 53% | 32% |
| 0.028% PC* | 600 | 59% | 782 | 31% | 18% |

*using 0.36% TiO₂ instead of 0.5% by weight

TABLE III
Results for the compound of example 3
in free-standing polycarbonate film,

| | Excitation | | | Abs |
|---|---|---|---|---|
| | wavelength/nm | QY | lambdaₘₐₓ/nm | int. |
| 0.005% PC | 660 | 42% | 764 | 28% |
| 0.1% PC | 660 | 28% | 805 | 28% |

TABLE IV
Result for the compound of comparative example
1 in free-standing polycarbonate film

| | Excitation | | | Abs | |
|---|---|---|---|---|---|
| | wavelength/nm | QY | lambdaₘₐₓ/nm | int. | Fluo. |
| 0.039% PC | 600 | 55% | 756 | 59% | 32% |
| 0.039% PC | 560 | 55% | 758 | 40% | 22% |
| 0.062% PC | 600 | 50% | 759 | 66% | 33% |
| 0.062% PC | 560 | 51% | 760 | 48% | 24% |

TABLE V
Result for the compound of comparative example
2 in free-standing polycarbonate film

| | Excitation | | | Abs | |
|---|---|---|---|---|---|
| | wavelength/nm | QY | lambdaₘₐₓ/nm | int. | Fluo. |
| 0.063% PC | 660 | 46% | 785 | 74% | 34% |
| 0.06% PC | 600 | 46% | 785 | 74% | 34% |
| 0.2% PC | 600 | 26% | 785 | 82% | 21% |
| 0.5% PC | 600 | 7% | 785 | 91% | 6% |
| 1% PC | 600 | 5% | 785 | 95% | 5% |

The quantum yield decreases at higher concentration due to aggregate formation and reabsorption effects.
The results show that the novel terrylene bisimide compounds can be used in higher concentration while still retaining a high quantum yield compared to the compounds of prior art.

### V. Application Examples

### V.1 Materials Used

Laromer® PE 46T of BASF SE is a commercial polyester acrylate in tripropylene glycol diacrylate (30%);
Laromer® EA 9081 of BASF SE is a commercial aromatic epoxy acrylate in monomer mix (75%);
Laromer® LR 8986 of BASF SE is a commercial aromatic epoxy acrylate;
Laromer® UA 9048 of BASF SE is a commercial aliphatic urethane acrylate in dipropylene glycol diacrylate (25%);
Laromer® UA 9072 of BASF SE is a commercial aliphatic urethane acrylate in 4-t-bu-tylcyclohexylacrylate (30%);
Laromer® DPHA of BASF SE is a commercial dipentaerythritol hexaacrylate;
Laromer® DPGDA of BASF SE is a commercial dipropylene glycol diacrylate;
Laromer® LR 8887 of BASF SE is a commercial trimethylolpropaneformal monoacry-late;
Vinnol® E 15/48 A of Wacker, Germany, is a commercial available hydroxyl-containing copolymer of approx. 84 wt. % vinyl chloride (VC) and approx. 16 wt. % of acrylic acid esters;
Omnirad® 184 of IGM Resins, Netherlands, is 1-hydroxycyclohexyl-phenyl ketone;
Omnirad® 2959 of IGM Resins, Netherlands, is 1-[4-(2-hydroxyethoxyl)-phenyl]-2-hy-droxy-2-methylpropanone;
   V.2 Preparation of inks

### Example 1: UV-Ink

0.1 g of the compound of example 1, 100 g of Laromer® PE 46T and 3 g diphe-nyl(2,4,6-trimethylbenzoyl)phosphinoxide (or ethyl(2,4,6-trimethylbenzoyl)phenyl-phosphinate) were mixed in a speedmixer apparatus in a 60 mL jar. A drawdown was prepared with a 12 (or 24) μm spiral wirebar on PET foil. The coated PET foil was cured 3 times at 120 W/cm² under a UV belt (Technigraf Aktiprint T/e with Hg-lamp (UT 51072-0004)). The achieved film showed an emission in the wavelength range from 700 to 1000 nm.

### Example 2: UV-Ink

Example 2 was prepared according to the general procedure of example 1, but using Laromer® EA 9081 instead of Laromer® PE 46T.

### Example 3: UV-Ink

Example 3 was prepared according to the general procedure of example 1, but using Laromer® LR 8986 instead of the Laromer® PE 46T.

### Example 4: UV Ink

0.05 g of the compound of example 4 was dissolved in 3.5 g of Laromer® DPGDA. 1.5 g of Omnirad® 184 was dissolved in 3.5 g of Laromer® DPGDA. Both solutions were added to 40 g of Laromer® UA 9048 and mixed either by hand or in a speedmixer equipment. A drawdown with a 24 μm spiral wirebar on a PET film was cured with a Technigraf Aktiprint T/e with Hg-lamp (UT 51072-0004) at 120 W/cm² with 20 m/min or 2×40 m/min at 120 W/cm²). The obtained films showed an absorption at 780 nm.

### Example 5: UV-Ink

Example 5 was prepared according to the general procedure of example 4, but using Omnirad®2959 instead of the Omnirad®184.

### Example 6: UV Ink

0.125 g of the compound of example 3, 100 g Laromer® UA 9072, 25 g of Laromer® DPGDA and 6.25 g of diphenyl(2,4,6-trimethylbenzoyl)phosphinoxide were mixed in a Speedmixer DAC 400.1 FVZ in a 60 mL jar with 5 glass beads with a 4 mm diameter for 2×140 minutes. The draw down with 12 μm spiral wirebar on Lumirror 4001/50 μm showed after UV curing (3*120 W/cm²) a fluorescence in the NIR.

### Example 7: UV Ink

Example 7 was prepared according to the general procedure of example 6, but using Laromer® LR 8887 instead of the Laromer® DPGDA.

### Example 8: UV Ink

Example 8 was prepared according to the general procedure of example 6, but using Laromer® DPHA instead of Laromer® UA 9072.

### Example 9—Offset Ink

An offset ink was prepared by mixing the following components by means of an automatic pigment muller: 1000 nm of Offset varnish, 200 mg of trithiocyanuric acid, 40 mg of the compound of example 4, 20 mg of siccative. Immediately afterwards the freshly prepared offset ink was printed onto uncoated paper with a printability tester (IGT Orange Proofer). The print showed an absorption with two maxima at 710 and 790 nm and by excitation at 660 nm an emission at 980 nm.

### Example 10—Vinylchloride Copolymer Ink

0.5 g of the compound of example 3 in 39.5 g of VINNOL E 15/48A (10% in ethyl ace-tate/methyl ethyl ketone/methyl isobutyl ketone=20:30:47) was dispersed in a 100 mL glass jar with 50 g of glass beads with a diameter of 2 mm for 30 minutes in the scan-dex. With a 24 μm spiral wirebar a drawdown was prepared on acetate film. The film showed an absorption at 720 nm.

1.-17. (canceled)

18. A compound of the formula (I) 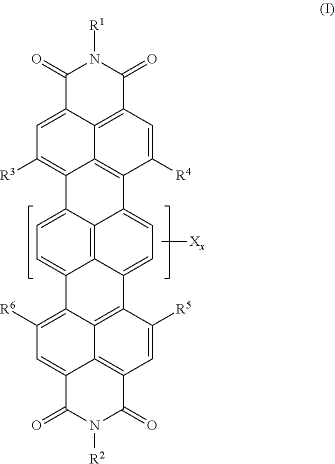
wherein
n is 1 or 2;
x is 0, 1 or 2;
each X is C₆-C₁₀-aryloxy or C₆-C₁₀-aryl, wherein C₆-C₁₀-aryloxy is substituted by one substituent R⁷ and one or more identical or different substituents R^{7a}; and wherein C₆-C₁₀-aryl is substituted by one substituent R⁸ and zero;
R¹ and R², independently of each other, are hydrogen, C₁-C₂₄-alkyl, C₁-C₂₄-haloalkyl, C₃-C₂₄-cycloalkyl, C₆-C₁₀-aryl or C₆-C₁₀-aryl-C₁-C₁₀-alkylene, where the rings of cycloalkyl, aryl, and aryl-alkylene in the three last-mentioned radicals are unsubstituted or substituted with one or more substituents R^{a}, and where C₁-C₂₄-alkyl, C₁-C₂₄-haloalkyl and the alkylene moiety of C₆-C₁₀-aryl-C₁-C₁₀-alkylene may be interrupted by one or more heteroatoms or heteroatomic groups selected from the group consisting of O, S and NR^{b};
R³, R⁴, R⁵ and R⁶, independently of each other, are C₆-C₁₀-aryloxy or C₆-C₁₀-aryl, wherein C₆-C₁₀-aryloxy is substituted by one substituent R⁷ and one or more substituents R^{7a} and wherein C₆-C₁₀-aryl is substituted by one substituent R^{g} and zero;
R⁷ is C₆-C₂M-alkyl;
each R^{7a} is independently C₁-C₂M-alkyl;
R^{g} is C₅-C₂M-alkyl;
each R^{8a} is independently C₁-C₂M-alkyl;
each R^{a} is independently selected from the group consisting of C₁-C₂₄-alkyl, C₁-C₂₄-fluoroalkyl, C₁-C₂₄-alkoxy, fluorine, chlorine and bromine; and
R^{b} is hydrogen, C₁-C₂M-alkyl, C₃-C₂₄-cycloalkyl, heterocycloalkyl, hetaryl or C₆-C₁₀-aryl.

19. The compound according to claim 18, wherein R¹ and R², independently of each other, are selected from the group consisting of linear C₁-C₂₄-alkyl, branched C₃-C₂₄-alkyl, C₅-C₈-cycloalkyl, phenyl and phenyl-C₁-C₁₀-alkylene, where the rings of cycloalkyl, phenyl and phenyl-alkylene in the three last-mentioned radicals are unsubstituted or substituted by 1, 2 or 3 identical or different substituents R^{a}.

20. The compound according to claim 18, wherein R³, R⁴, R⁵ and R⁶, independently of each other, are phenoxy, which is substituted by one substituent R⁷ and by one or two substituents R^{7a}, and wherein R⁷ is attached to phenoxy in para position and at least one of the substituents R^{7a} is attached to phenoxy in the ortho position and wherein R⁷ is linear or branched C₆-C₁₂-alkyl and wherein each R^{7a} is independently linear C₁-C₁₀-alkyl or branched C₃-C₁₀-alkyl; or
R³, R⁴, R⁵ and R⁶, independently of each other, are phenyl which is substituted by one substituent R^{g} and 0, 1 or 2 substituents R^{8a}, wherein R^{g} is linear or branched C₅-C₁₂-alkyl and wherein each R^{8a}, if present, is C₁-C₁₀-alkyl.

21. The compound according to claim 18, wherein each X is independently phenoxy, which is substituted by one substituent R⁷ and by one or two substituents R^{7a}, wherein R⁷ is attached to phenoxy in para position and at least one of the substituents R^{7a} is attached to phenoxy in the ortho position and wherein R⁷ is linear or branched C₆-C₁₂-alkyl and wherein each R^{7a} is independently linear C₁-C₁₀-alkyl or branched C₃-C₁₀-alkyl; or
each X is independently phenyl which is substituted by one substituent R⁸ and zero, one or two substituents R^{8a} and wherein R⁸ is linear or branched C₅-C₁₂-alkyl and wherein each R^{8a}, if present, is C₁-C₁₀-alkyl.

22. The compound of the formula (Ia) according to claim 18 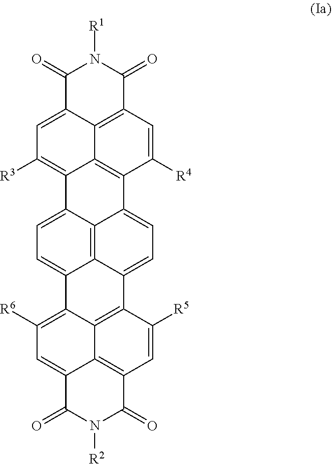
wherein
R¹ and R² have the same meaning and are selected from C₄-C₂₀-alkyl, C₅-C₈-cycloalkyl or phenyl, wherein the two last-mentioned substituents are substituted by one, two or three C₁-C₆-alkyl substituents; and
R³, R⁴, R⁵ and R⁶ have the same meaning and are selected from the group consisting of phenoxy and phenyl; wherein phenoxy is substituted by one substituent R⁷ and one or two substituents R^{7a}; wherein R⁷ is attached to phenoxy in para position and at least one of the substituents R^{7a} is attached to phenoxy in the ortho position, and wherein R⁷ is linear or branched C₆-C₁₂-alkyl and each R^{7a} is independently linear C₁-C₁₀-alkyl or branched C₃-C₁₀-alkyl; and wherein phenyl is substituted by one substituent R′, wherein
R⁸ is linear or branched C₅-C₁₂-alkyl.

23. The compound of the formula (Ib) according to claim 18 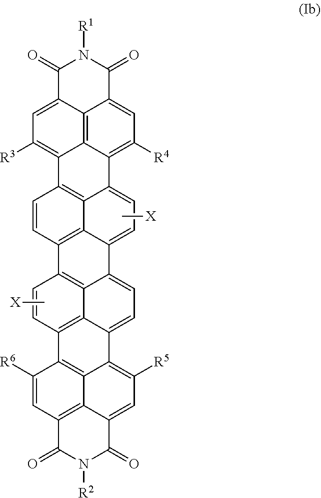
wherein
R¹ and R² have the same meaning and are selected from the group consisting of C₄-C₂M-alkyl, C₅-C₈-cycloalkyl and phenyl, wherein the two last-mentioned substituents are substituted by one, two or three C₁-C₆-alkyl substituents;
R³, R⁴, R⁵ and R⁶ have the same meaning and are phenoxy, which is substituted by one substituent R⁷ and one or two substituents R^{7a}; and
each X is independently phenoxy, which is substituted by one substituent R⁷ and one or two substituents R^{7a},
wherein in each case R⁷ is attached to phenoxy in para position and at least one of the substituents R^{7a} is attached to phenoxy in the ortho position, and wherein R⁷ is linear or branched C₆-C₁₂-alkyl and each R^{7a} is independently linear C₁-C₁₀-alkyl.

24. The compound of formula (I) according to claim 18, where R¹ and R² have the same meaning and are phenyl substituted by one, two, or three C₁-C₆-alkyl substituents, by one or two branched C₃-C₆-alkyl substituents.

25. The compound of formula (I) according to claim 18, where R³, R⁴, R⁵, R⁶ and X, if present, have the same meaning and are selected from the group consisting of phenoxy and phenyl; wherein phenoxy is substituted by one substituent R⁷ and one or two substituents R^{7a};
wherein R⁷ is attached to phenoxy in para position and the one or two substituents R^{7a} are attached to phenoxy in the ortho position; wherein R⁷ is branched C₆-C₁₂-alkyl; and R^{7a} is branched C₃-C₁₀-alkyl; and wherein phenyl is substituted by one substituent R′, wherein R′ is linear C₅-C₁₂-alkyl.

26. The compound of formula (I) according to claim 18 selected from the group consisting

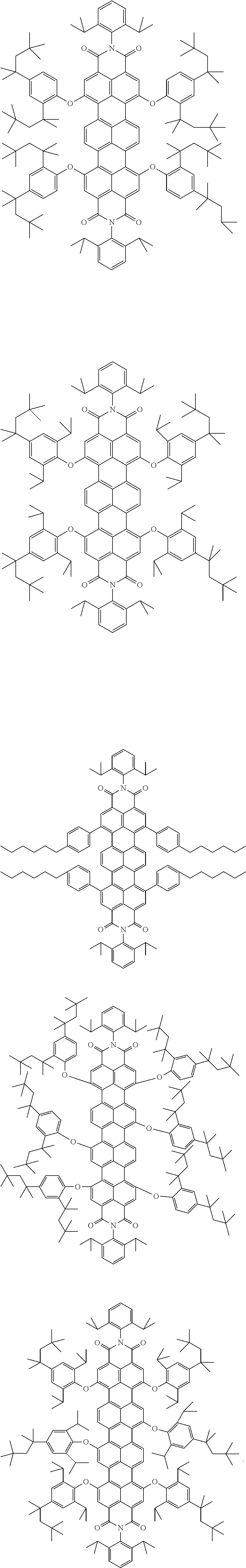

27. A method for security printing, for coloring coatings, printing inks including printing inks for 3D printing and plastics, for data storage, for optical labels, for security labels in documents, for brand protection, for solar collectors, for optical waveguides, for the laser welding of plastics or as a fluorescent label for biomolecules comprising utilizing the compound of formula (1) as defined in claim 18.

28. An article comprising at least one film layer, the film layer comprising in a polymeric matrix the compound of formula (I) as defined in claim 18.

29. A printing ink formulation for security printing, comprising a) at least one compound of formula (I) as defined in claim 18;
b) a polymeric binder;
c) optionally a solvent;
d) optionally at least one colorant;
e) optionally at least one further additive; and
f) optionally at least one photoinitiator.

30. The printing ink formulation according to claim 29, comprising
a) 0.0001 to 25% by weight of at least one compound of formula (I);
b) 5 to 99.9999% % by weight of at least one polymeric binder;
c) 0 to 94.9999% by weight of at least one solvent;
d) 0 to 25% by weight of at least one colorant;
e) 0 to 25% by weight of at least one further additive; and
f) 0 to 14% by weight at least one photoinitiator;
wherein the sum of components a) to f) adds up to 100%.

31. A security document, comprising a substrate and the compound of formula (I) as defined in claim 18.

32. A security document, obtained by a printing process, wherein the printing ink formulation as defined in claim 29 is employed.

33. The security document according to claim 31, wherein the security document is selected from a bank note, a passport, a check, a voucher, an ID- or transaction card, a stamp and a tax label.

34. A method of detecting the authenticity of the security document as defined in claim 31, comprising the steps of:
a) measuring an absorbance, reflectance or transmittance spectrum of the security document in the visible/near infra-red range of the electromagnetic spectrum; and
b) comparing the spectrum measured under a) and/or information derived therefrom with a corresponding spectrum and/or information of an authentic security element.
